(19) United States
(12) Patent Application Publication
(10) Pub. No.: US 2022/0033774 A1
(43) Pub. Date: Feb. 3, 2022

(54) COMPOSITIONS AND METHODS FOR CYTOTOXIC CD4+ T CELLS

(71) Applicant: The Regents of the University of California, Oakland, FL (US)

(72) Inventors: David OH, San Francisco, CA (US); Lawrence FONG, San Francisco, CA (US); Serena KWEK MACPHEE, San Francisco, CA (US); Chun Jimmie YE, San Francisco, CA (US); Chien-Chun Steven PAI, San Francisco, CA (US)

(21) Appl. No.: 17/047,975

(22) PCT Filed: Apr. 16, 2019
(86) PCT No.: PCT/US19/27723

§ 371 Date: Oct. 15, 2020

Related U.S. Application Data
(60) Provisional application No. 62/658,568, filed on Apr. 16, 2018.

(51) Int. Cl. C12N 5/0783; A61K 35/17

(52) U.S. Cl. CPC C12N 5/0638; C12N 2501/2302; A61K 35/17

(57) ABSTRACT

Provided herein, inter alia, are compositions and methods for treating bladder cancer, including CD4+T cells that can be cultured ex vivo to generate a population of cytotoxic CD4⁺T cells capable of killing bladder cancer tumor cells. Pharmaceutical compositions containing such a cytotoxic CD4⁺T cell population, as well as methods for treating an individual having or suspected of having bladder cancer are also provided.

### CROSS-REFERENCE TO RELATED APPLICATIONS

This application claims the benefit of priority to U.S. Provisional Patent Application Ser. No. 62/658,568, filed on Apr. 16, 2018, which is herein expressly incorporated by reference it their entireties, including any drawings.

### GOVERNMENT INTEREST

This invention was made with Government support under grant no. R01 CA194511 awarded by the National Institutes of Health. The Government has certain rights in the invention.

### INCORPORATION OF THE SEQUENCE LISTING

This application contains a Sequence Listing which has been submitted electronically in ASCII format and is hereby incorporated by reference in its entirety. The ASCII copy, created on Apr. 9, 2019, 2019, is named 048536-623001WO_SL_ST25.txt, and is 4.91 kb bytes in size.

### FIELD

Disclosed herein, inter alia, are methods and compositions for the treatment of bladder cancer, including novel populations of CD4⁺ T cells that can be cultured ex vivo to generate a population of cytotoxic CD4⁺ T cells capable of killing bladder cancer tumor cells.

### BACKGROUND

Immunotherapies have changed the landscape of cancer treatment by producing durable and long-lasting responses through triggering of anti-tumor cell-mediated immunity. In particular, checkpoint inhibitors (CPI) targeting immune inhibitory molecules CTLA-4 and PD-1 in T lymphocytes have been approved based on responses and improved overall survival in multiple malignancies, particularly those with high mutational burden. However, it has been reported that even in the most responsive malignancies, CPIs as monotherapies are efficacious in only ˜20% of patients. Without being bound to theory, this could be partly due to the heterogeneity of tumor-infiltrating T lymphocytes (TILs) and their differential response to treatment to confer a therapeutic benefit.

Bladder cancer can be responsive to immunotherapies such as anti-PD-1 and anti-PD-L1 checkpoint inhibitors, which are believed to relieve inhibition of cytotoxic CD8⁺ T cells resulting in tumor cell killing. However, although immunotherapies such as anti-PD-1 and anti-PD-L1 checkpoint inhibitors have shown some promise in treating bladder cancer, the overall response rates have remained low. In addition, while cytotoxic CD8⁺ T cells are thought to mediate tumor rejection, the contribution of other tumor-resident T cells, which may possess heterogeneity in their antigenic repertoire and function, is unknown.

Hence, there remains a need for additional therapeutic approaches, leading to more effective treatments to target and kill bladder cancer cells.

### SUMMARY

The present disclosure provides, inter alia, novel compositions and methods for treating bladder cancer. In particular, described herein are a novel populations of CD4⁺ T cells that can be cultured ex vivo to generate a population of cytotoxic CD4⁺ T cells capable of killing bladder cancer tumor cells. Also provided are methods for producing a population of cytotoxic CD4⁺ T cells with cytolytic capabilities, and pharmaceutical compositions containing such a population of cytotoxic CD4⁺ T cells, as well as methods for (i) treating an individual having or suspected of having bladder cancer, (ii) targeting cancer cells in an individual having or suspected of having bladder cancer, and (iii) providing cell therapy to an individual having or suspected of having bladder cancer

In one aspect, there is provided an ex vivo population of CD4⁺ T cells wherein the CD4⁺ T cells express one or more markers selected from the group consisting of GZMA, GZMB, GZMH, GZMK, KLRB1, KLRD1, GNLY, NKG7, CCL4, CCL5, LTB, CXCR4, CXCR6, PRF1, KLRG1, LAG3, CXCL13, and combinations of any thereof, and wherein the CD4⁺ T cells have cytolytic capabilities.

Implementations of embodiments of the ex vivo population of CD4⁺ T cells of the disclosure can include one or more of the following features. In some embodiments, the CD4⁺ T cells express the immune checkpoint marker LAG3 but lack expression of one or more additional immune checkpoint markers. In some embodiments, the additional immune checkpoint markers not expressed by the CD4⁺ T cells are selected from the group consisting of IL2RA/CD25, TNFRSF4/OX40, TNFRSF9/4-1BB, TNFRSF18/GITR, CD278/ICOS, TIGIT, and combinations of any thereof. In some embodiments, the CD4⁺ T cells further express heat shock proteins and/or IFN-gamma. In some embodiments, the cytolytic capabilities are detected by lysis of autologous tumor cells, allogeneic human tumor cells, or mouse mastocytoma target cells (P815) loaded with anti-human CD3 antibodies. In some embodiments, the CD4⁺ T cells express a T cell receptor (TCR) including (i) a TCR alpha CDR3 sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9; and (ii) a TCR beta CDR3 sequence selected from the group consisting of SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, and SEQ ID NO: 19. In some embodiments, the CD4⁺ T cells are obtained from a biological sample including bladder cancer cells. In some embodiments, the CD4⁺ T cells are obtained from a biological sample including peripheral blood from an individual having or suspected of having bladder cancer. In some embodiments, the CD4⁺ T cells have decreased cytolytic capabilities as compared to the CD4⁺ T cells which have been expanded ex vivo. In some embodiments, the population is at least 50%, 60%, 70%, 80%, 90%, or 95% enriched for cytolytic CD4⁺ T cells. In some embodiments, the CD4⁺ T cells are capable of killing autologous cancer cells.

In one aspect, provided herein is a pharmaceutical composition for cell therapy, the composition including a population of CD4⁺ T cells as described herein and a pharmaceutically acceptable excipient. In one aspect, provided herein is a pharmaceutical composition for cell therapy, the composition including a population of CD4⁺ T cells with cytolytic capabilities and a pharmaceutically acceptable excipient. In some embodiments, the population is at least 50%, 60%, 70%, 80%, 90%, or 95% enriched for CD4⁺ T cells with cytolytic capabilities.

In another aspect, provided herein is a method of producing an ex vivo expanded population of CD4⁺ T cells with cytolytic capabilities, the method including: (a) separating CD4⁺ T cells from a biological sample containing a mixture of different types of immune cells; (b) culturing the separated CD4⁺ T cells in media containing IL-2 in an amount sufficient to promote the expansion of CD4⁺ T cells; and (c) splitting the cultured CD4⁺ T cells to promote the enrichment of CD4⁺ T cells thus producing an ex vivo expanded population of CD4⁺ T cells with cytolytic capabilities. In some embodiments, IL-2 is used in the amount of about 1 IU/ml to about 2000 IU/ml. In some embodiments, CD8⁺ cells are removed from the mixture in step (a). In some embodiments, the enriched population of CD4⁺ T cells with cytolytic capabilities express one or more markers selected from the group consisting of GZMA, GZMB, GZMH, GZMK, KLRB1, KLRD1, GNLY, NKG7, CCL4, CCL5, LTB, CXCR4, CXCR6, PRF1, KLRG1, LAG3, and CXCL13. In some embodiments, the CD4⁺ T cells express the immune checkpoint marker LAG3 but lack expression of one or more additional immune checkpoint markers. In some embodiments, the additional immune checkpoint markers not expressed by the CD4⁺ T cells are selected from the group consisting of IL2RA/CD25, TNFRSF4/OX40, TNFRSF9/4-1BB, TNFRSF18/GITR, CD278/ICOS, TIGIT, and combinations of any thereof. In some embodiments, the CD4⁺ T cells further express heat shock proteins and/or IFN-gamma. In some embodiments, the cytolytic capabilities are detected by lysis of autologous tumor cells, allogeneic human tumor cells, or mouse mastocytoma target cells (P815) loaded with anti-human CD3 antibodies.

In one aspect, provided herein is a method of treating an individual having or suspected of having bladder cancer, the method including administering to the individual an effective amount of a population of cytolytic CD4⁺ T cells. In a further aspect, provided herein is a method of targeting cancer cells in an individual having or suspected of having bladder cancer, the method including administering to the individual an effective amount of a population of cytolytic CD4⁺ T cells. In a further aspect, provided herein is a method of providing cell therapy to an individual having or suspected of having bladder cancer, the method including administering to the individual an effective amount of a population of cytolytic CD4⁺ T cells. In some embodiments, one or more bladder cancer cells is destroyed. In some embodiments, the population of cytolytic CD4⁺ T cells to be administered to the individual is an ex vivo population of cytolytic CD4⁺ T cells. In some embodiments, the CD4⁺ T cells express one or more markers selected from the group consisting of GZMA, GZMB, GZMH, GZMK, KLRB1, KLRD1, GNLY, NKG7, CCL4, CCL5, LTB, CXCR4, CXCR6, PRF1, KLRG1, LAG3, CXCL13, and combinations of any thereof, and wherein the CD4⁺ T cells have cytolytic capabilities. In some embodiments, the CD4⁺ T cells express the immune checkpoint marker LAG3 but lack expression of one or more additional immune checkpoint markers. In some embodiments, the additional immune checkpoint markers not expressed by the CD4⁺ T cells are selected from the group consisting of IL2RA/CD25, TNFRSF4/OX40, TNFRSF9/4-1BB, TNFRSF18/GITR, CD278/ICOS, TIGIT, and combinations of any thereof. In some embodiments, the CD4⁺ T cells further express heat shock proteins and/or IFN-gamma.

Each of the aspects and embodiments described herein are capable of being used together, unless excluded explicitly or clearly from the context of the embodiment or aspect.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIGS. 1A-1B show an overview of the experimental approach, and demonstration of CD4⁺ enrichment over CD8⁺ in muscle-invasive bladder cancer. FIG. 1A graphically summarizes the parallel flow cytometry data from the same single-cell digest used for droplet-based single-cell RNA sequencing (dscRNAseq) from 4 anti-PD-L1-treated tumors and paired adjacent normal tissues, showing the percentage of CD4⁺ or CD8⁺ T cells from total CD3⁺ cells. FIG. 1B is a schematic diagram of the processing for paired tumor, adjacent non-malignant tissue, and blood from either anti-PD-L1-treated, or untreated/chemotherapy-treated, cystectomy patients. FACS-sorted CD4⁺ or CD8⁺ T cells were subjected to dscRNAseq with paired T cell receptor (TCR) sequencing as described in the text. Subsequently, cell populations that are specific to “tumor” can be identified when compared to paired “normal”/“non-malignant” tissue and blood from the same patient. Comparison can also be performed across patients and treatments, such as chemotherapy and anti-PD-L1 therapy.

FIGS. 2A-2E graphically summarize the results of experiments showing that CD4⁺ T cells in bladder tumors are composed of multiple populations of regulatory and cytotoxic CD4⁺ cells. In particular, intratumoral CD4⁺ T cells include regulatory T cells (Tregs) and several populations of novel cytotoxic CD4⁺. FIG. 2A shows a pooled analysis of single-cell RNA sequencing (scRNAseq) data from 22,000 CD4⁺ tumor infiltrating lymphocytes, represented on a two-dimensional t-Distributed Stochastic Neighbor Embedding (t-SNE) plot. Unbiased clustering revealed distinct functional populations including several cytotoxic CD4⁺ populations (tCD4-c4, tCD4-c7, tCD4-c9, tCD4-c10). In these experiments, t-Distributed Stochastic Neighbor Embedding (tSNE) plots of 21,932 single sorted CD3⁺CD4⁺ T cells were obtained from bladder tumors and adjacent non-malignant tissue from 7 patients. At left, each distinct phenotypic cluster identified using graph-based k-nearest neighbor (KNN) methods (Seurat) is identified with a distinct color. At right, the sample of origin of the same cells (tumor or non-malignant) is indicated by appropriate colors (blue or red, respectively). Annotation of each unbiased cluster was performed by manual inspection of highest-ranked differentially expressed genes for each cluster, and also using reference signature-based correlation methods (SingleR) as described in the text. FIG. 2B illustrates relative intensity of expression of select genes superimposed upon the tSNE projections shown in FIG. 2A. Novel cytolytic CD4⁺ populations expressed GZMB, GZMK, or both and did not express immune checkpoints such as CD25 (IL2RA) and TNFRSF18 (GITR), as shown by relative expression within the cytotoxic populations superimposed on the tSNE plot from FIG. 2A. FIG. 2C is heatmap data showing distinct modules of genes that were differentially expressed in CD4⁺ subpopulations including cytotoxic CD4⁺ (tCD4-c4, tCD4-c7, tCD4-c9, tCD4-c10). Heatmap showing all single cells (columns) grouped by the unbiased clusters shown in FIG. 2A and by tissue of origin (colors at top of heatmap), with relative expression of the top 10 ranked differentially expressed genes for each cluster compared to the CCR7⁺ tCD4-c1 cluster (genes as rows, ordered by fold change, all P_{adj} values <0.05) displayed. FIG. 2D shows violin plots of select marker genes that were differentially expressed between regulatory subpopulations, and between cytotoxic CD4⁺ subpopulations. FIG. 2E is flow cytometry data confirming that cytotoxic CD4⁺ expressed GZMB, GZMK, or both at the protein level, and did not express immune checkpoints such as CD25 (IL2RA) and TNFRSF18 (GITR) (also see, e.g., FIGS. 7A-7B).

FIGS. 3A-3B show that CXCL13 and IFNG were expressed specifically in tumor-resident CD4⁺ T cells compared to normal tissue. FIG. 3A is heatmap data showing gene expression levels of CD4⁺ cells obtained from tumor or normal tissue specifically for the tCD4-c3 population. FIG. 3B shows relative expression of CXCL13 and IFNG are superimposed on the tSNE plots from FIG. 2A, shown for CD4⁺ obtained from tumor or normal tissue.

FIGS. 4A-4C show that intratumoral CD8⁺ T cells in bladder tumors include known populations of MAIT, effector, central memory, and cycling T cells. FIG. 4A shows tSNE plots of 11,794 single sorted CD3⁺CD8⁺ T cells obtained from bladder tumors and adjacent non-malignant tissue from 7 patients. Phenotypic clusters (left) and compartment of origin (tumor and non-malignant are blue and red, right) are shown as in FIG. 2A. FIG. 4B shows relative intensity of expression of select genes superimposed upon the tSNE projections shown in FIG. 4A. FIG. 4C is heatmap data showing all single cells (columns) grouped by the unbiased clusters shown in FIG. 4A and by tissue of origin (colors at top of heatmap), with relative expression of the top 10 ranked differentially expressed genes for each cluster compared to the CCR7⁺ tCD8-c0 cluster and conserved across tumor and non-malignant compartments based on meta testing (genes as rows, ordered by fold change, all P_{adj} values <0.05) displayed. Select marker genes were identified that were differentially expressed between subpopulations based on pairwise population testing.

FIGS. 5A-5D graphically summarize the results of experiments performed to show that the intratumoral CD4⁺ compartment exhibits a predominance of regulatory T cells as well as enrichment of activation markers. FIGS. 5A-5B: For each CD4⁺ (5A) or CD8⁺ (5B) cluster, the abundance of cells is shown as a percentage of all cells within either tumor or non-malignant compartments across all patients (orange=tumor, blue=non-malignant). FIG. 5C shows the ratio of abundances of all regulatory T cell populations (tCD4-c0+tCD4-c5+tCD4-c6) to all cytotoxic CD4⁺ populations (tCD4-c4+tCD4-c7+tCD4-c9+tCD4-c10) across all tumor and non-malignant samples. For parts (A-C), *, P<0.05, **, P<0.01 and FDR<0.1 by unpaired two-tailed T test assuming unequal variances with Benjamini-Hochberg correction for multiple testing. FIG. 5D shows scatter plots of average gene expression in tumor and non-malignant compartments for the specific CD4⁺ clusters. Genes that are significantly different with P_{adj}<0.05 are shown in black. Significantly different genes that demonstrate an absolute log₂ fold-change of greater than 1 between tumor and normal are shown in red.

FIGS. 6A-6J graphically summarize the results of experiments performed to illustrate that intratumoral cytotoxic CD4⁺ are clonally expanded in bladder tumors and modulated by anti-PD-L1 therapy. FIG. 6A shows the percentage of unique paired TRA and TRB CDR3 nucleotide sequences that are expressed by one cell (blue), shared by two cells (green), or shared by three or more cells (red) is indicated for CD4⁺ T cells from individual tumor and non-malignant tissues from anti-PD-L1-treated (“PD-L1”), untreated, and chemotherapy-treated (“chemo”) patients. Triplicate control samples from a single healthy donor's CD4⁺ T cells sorted from peripheral blood and processed for scRNAseq and TCR in identical fashion in separate sequencing runs is shown (“healthy 1-3”), as well as reference publicly available data from peripheral blood CD4⁺ from a healthy donor. FIG. 6B shows Lorenz curves showing the cumulative frequency distributions for unique CD4⁺ cells and CD4⁺ clonotypes for tumor, non-malignant tissues, and healthy donor blood. FIG. 6C shows Gini coefficients for CD4⁺ clonotypes from tumor, non-malignant tissues, and healthy donor blood, calculated from the Lorenz curves in FIG. 6B. P=0.005 by Wilcoxon for tumor versus non-malignant tissues. FIGS. 6D-6F show paired TRA/TRB clonotype sharing between cells, Lorenz curves, and Gini coefficients for CD8⁺ clonotype data as in parts A-C. FIG. 6G shows Gini coefficients for each of the CD4⁺ populations within tumor and non-malignant compartments across all samples (*, P<0.05, **, P<0.01 and FDR<0.1 by Wilcoxon test with Benjamini-Hochberg correction for multiple testing). FIG. 6H is a circle plot showing all unique paired TRA/TRB clonotypes from CD4⁺ cells that are shared between unique cells from distinct phenotypic populations. Each population is represented by a distinctly colored arc within the circle edge; the widths of each segment of the circle and the arcs connecting populations are proportional to clonotype frequency. FIGS. 6I-6J are heatmap data showing empirical P values for observed sharing of the same unique paired TRA and TRB clonotype CDR3 nucleotide sequences between 2 phenotypic populations within tumor (FIG. 6I) or between tumor and non-malignant tissue (FIG. 6J), in the total sample set (left panels) or within the subset of anti-PD-L1-treated samples (right panels). Red indicates pairs of populations with an empirical P value <0.01. A black cross indicates pairs of populations with significant sharing only in the total sample set, while a white cross indicates significant sharing only seen in anti-PD-L1-treated samples.

FIGS. 7A-7E graphically summarize the results of experiments performed to illustrate that cytotoxic CD4⁺ T cells from bladder tumors are functional. FIG. 7A shows a flow cytometry analysis of GZMB and GZMK expression within central memory (CCR7⁺ CD45RA⁻), effector memory (CCR7⁻ CD45RA⁻), or effector (CCR7⁻ CD45RA⁺) subgroups of CD8⁺, CD4⁺ FOXP3⁻, or CD4⁺ FOXP3⁺ populations from tumor-infiltrating lymphocytes obtained from a bladder tumor that was not treated with systemic therapy before cystectomy. FIG. 7B shows a flow cytometry analysis of CD3 expression versus immune checkpoints (PD-1, TIGIT), NKG7, and CD25 within CD4⁺ FOXP3⁺ and specific CD4⁺ FOXP3⁺ populations expressing GZMB and/or GZMK. FIG. 7C shows specific timepoints from a time-lapse microscopy experiment where sorted CD4⁺ TIL (with regulatory T cells excluded) from a localized bladder tumor were isolated, cultured ex vivo (see Methods), and re-incubated with autologous tumor cells at a effector:target ratio of approximately 30:1 at timepoint 0. Timepoints involving recognition of tumor by TILs (as evidenced by cluster formation), and killing (with increase in uptake of red cell death indicator) are displayed at the indicated times. FIG. 7D shows an analysis of the increase in the number of dead cells over time from the same killing assay for CD4⁺ TIL at different effector:target ratios (top) or with MHCII blockade (bottom). Control traces from the same wells but restricting analysis to free CD4⁺ TIL, or from separate wells with tumor only, are included. All traces were normalized to the number of dead cells per mm² at timepoint 0. FIG. 7E shows data from CD8⁺ TIL from the same killing assay run in parallel. The observation of autologous tumor killing within hours by CD4⁺ and CD8⁺ TIL above the background level of spontaneous death of TIL from the same wells is representative of 2 independent experiments involving distinct aliquots from the same patient.

FIGS. 8A-8G graphically summarize the results of experiments performed to illustrate that circulating CD4⁺ T cells are clonally expanded and include cytotoxic CD4⁺ T cells. FIG. 8A shows tSNE plots of phenotypic populations identified via unbiased clustering of 75,016 single sorted CD3⁺CD4⁺ T cells obtained from peripheral blood mononuclear cells (PBMC) from the 7 patients with scRNAseq data from bladder tumors, including 4 patients treated with anti-PD-L1 with blood before and after treatment, as well as 2 untreated patients and 1 chemotherapy-treated patient with blood taken at the time of cystectomy. Triplicate sorted PBMCs from a healthy donor processed in parallel, as well as reference data from 10× Genomics from a healthy donor for TCR-only data, were also included. FIG. 8B shows that sample of origin (anti-PD-L1-treated or “atezo”, anti-PD-L1-untreated or “SOC”, healthy) in tSNE space. FIG. 8C is heatmap data showing all single cells (columns) grouped by the unbiased clusters shown in FIG. 8A, showing relative expression of top ranked differentially expressed genes for each cluster versus the CCR7⁺ bCD4-c0 cluster (genes as rows, ordered by fold change, all P_{adj} values <0.05). FIG. 8D shows feature plots showing relative expression of marker genes superimposed on tSNE space. FIG. 8E shows fold change in abundance of cells within each phenotypic cluster after anti-PD-L1 treatment, shown for each individual patient who was treated. Responders are shown in blue and grey, non-responders in orange and yellow. FIG. 8F shows Gini coefficient across all samples from cancer patients and the healthy control samples detailed above. FIG. 8G shows Gini coefficient for each phenotypic population, across all cancer patient and healthy control samples. **, P<0.01 by Wilcoxon test (cancer versus healthy).

FIGS. 9A-9G graphically summarize the results of experiments performed to illustrate that circulating CD8⁺ T cells are clonally expanded. 50,421 single sorted CD3⁺CD8⁺ T cells were subjected to unbiased clustering, differential expression for marker genes, and quantitation of abundance by cluster and repertoire restriction by Gini coefficient as in FIGS. 8A-8G.

FIGS. 10A-10D show that a subset of tumor-infiltrating cytotoxic CD4⁺ circulate. FIG. 10A shows correlation matrix of all CD4⁺ populations from blood and tumor based on expression of shared genes. Populations within blood and tumor were arranged based on hierarchical clustering. FIG. 10B is a heatmap showing TCR sharing between pairs of CD4⁺ populations in blood and tumor where the empirical P value for sharing is less than 0.01, as in FIGS. 6I-6J. TCR sharing is shown for all samples and for the subset of anti-PD-L1-treated samples. FIGS. 10C-10D show correlation matrix for expression (C) and TCR sharing (D) between pairs of populations for CD8⁺ populations.

FIGS. 11A-11F show distinct phenotypic classes of cytotoxic CD4⁺ circulate in the periphery of bladder cancer patients, and may be selectively modulated by PD-1 blockade. FIG. 11A shows representative staining of peripheral blood mononuclear cells (PBMCs) from a patient with localized bladder cancer prior to anti-PD-L1 treatment on clinical trial, showing protein expression of GZMB and PRF1 (isotype control for PRF1 staining also shown). tSNE projection in FIG. 11B showing the phenotypic clusters identified by Phenograph, using the flow cytometric panel shown in the heatmap in FIG. 11C which also demonstrates expression of cytotoxic and other functional markers across stained PBMCs. A total of 14 paired pre-treatment and post-treatment samples (after PD-L1 treatment, near the time of surgery) from patients with localized bladder cancer treated on this trial, as well as PBMCs from 8 healthy donors, were used for clustering and analysis. FIGS. 11D-11F show abundance of specific cytotoxic populations (% of total) shown for healthy donors and pre/post-treatment timepoints for Cluster 5 (GZMB/NKG7/PRF1, also KLRB1/GZMK), Cluster 2 (GZMB/NKG7/PRF1/KLRG1, PD-1 negative), and Cluster 3 (GZMB/NKG7/PRF1/KLRG1, PD-1-expressing).

FIG. 12 graphically summarizes the results from an analysis that assigns each single cell to the best-known immune subset based on gene expression (SingleR). The analysis provides annotations of single CD4⁺ cells from tumor and adjacent non-malignant tissue using SingleR.

FIG. 13 shows a correlation matrix of all CD4⁺ and CD8⁺ populations from tissue (combined tumor and non-malignant tissues) based on expression of shared genes. In this figures, cell populations were arranged based on hierarchical clustering.

FIG. 14. Top panel: Gini coefficients for tissue-infiltrating CD4⁺ (top row) and CD8⁺ T cells (middle row) in individual populations, separated by tumor versus non-malignant tissue (left column) and treatment type (right column). Bottom panel: pairs of populations showing significant clonotype sharing are shown for tissue-infiltrating CD8⁺ within the tumor compartment for all samples (left), within tumor for anti-PD-L1-treated samples (middle), and between tumor and non-malignant tissues for all samples (right).

FIG. 15 summarizes the results of a flow cytometric analysis of expression of CD3, ICOS, CD127 for CD4_{reg} (FOXP3⁺), CD4⁺ non-cytotoxic cells (FOXP3⁻ GZMB/K⁻), and specific CD4_{cyto} populations (FOXP3⁻ GZMB⁺ and/or GZMK⁺).

FIG. 16 are tSNE plots showing cluster representation for circulating CD4⁺ and CD8⁺ from individual bladder cancer and healthy patients.

FIGS. 17A-17L show population metrics for circulating CD4⁺ and CD8⁺. FIGS. 17A-17B show abundance of single CD4⁺ cells per cluster by treatment (FIG. 17A), or comparing cancer versus healthy PBMC controls (FIG. 17B). FIGS. 17C-17D show Lorenz curves for cumulative frequency of CD4⁺ cells versus cumulative frequency of clones comparing tumor and non-malignant tissues (FIG. 17C) and treatment types (FIG. 17D). FIGS. 17E-17F show Gini coefficients for CD4⁺ cells separated by treatment type (combining all populations) (FIG. 17E) or by individual populations (FIG. 17F). FIGS. 17G-17J are similar to FIGS. 17A-17F but with circulating CD8⁺.

FIGS. 18A-18B graphically show abundance of single CD4⁺ T cells found in specific functional populations, within anti-PD-L1-treated tumor and non-malignant tissues (FIG. 18A) and in untreated tumor and non-malignant tissues (FIG. 18B).

### DETAILED DESCRIPTION

The present disclosure generally relates to, inter alia, compositions and methods for the treatment of bladder cancer. More particularly, the disclosure provides an ex vivo population of CD4⁺ T cells, wherein the CD4⁺ T cells express specific markers and have cytolytic capabilities. Also provided are methods for producing such CD4⁺ T cell populations, as well as pharmaceutical compositions for cell therapy containing such CD4⁺ T cell population. Additionally, the disclosure also provides (i) methods for treating an individual having or suspected of having bladder cancer, (ii) methods for targeting cancer cells in an individual having or suspected of having bladder cancer, and (iii) methods of providing cell therapy to an individual having or suspected of having bladder cancer.

Current efforts to dissect the mechanism of tumor immune surveillance and enhance efficacy of cancer immunotherapies have primarily focused on conventional cytotoxic CD8⁺ mediated response. However, given the known functional diversity of CD4⁺ effector responses, and emerging data that CD4⁺ recognition may be important for anti-tumor responses for instance in the context of a neoantigen vaccine, the role of specific CD4⁺ populations in enhancing or suppressing immune responses in the tumor microenvironment, and how these are modulated by systemic therapies including immunotherapy, remain unknown. As described in more detail below, unbiased massively parallel genotypic and phenotypic profiling of the T cell compartment in localized bladder tumors and the adjacent non-malignant compartment, including those treated with anti-PD-L1 immunotherapy, can be used to demonstrate several key conceptual advances in our understanding of tumor immunity.

Although immunotherapies such as anti-PD-1 and anti-PD-L1 checkpoint inhibitors have shown some promise in treating bladder cancer, the overall success rates have remained low. Tumor-resident T cells demonstrate considerable heterogeneity as far as antigenic repertoire and phenotype. Whether the bladder tumor environment is enriched for specific types of T cells with a particular functional profile and antigenic specificity, and whether these tumor-specific populations are associated with treatment or response to immunotherapy, remains unclear.

Currently, cytotoxic CD8⁺ T cells are the main focus of efforts to understand how immunotherapy elicits anti-tumor immunity. In melanoma, expression and chromatin state signatures of cytotoxicity and exhaustion and the presence of CD8⁺ T cells at the tumor invasive margin pre-treatment are significantly correlated with subsequent responses to PD-1-directed therapy. However, in metastatic transitional cell carcinoma (TCC) of the bladder, where response rates to PD-1 blockade are 15-20% in platinum-refractory patients and >20% in frontline platinum-ineligible patients, PD-L1 expression alone is poorly predictive. Recently, a detailed interrogation of the pre-treatment tumor microenvironment in TCC found that a higher score of CD8⁺ gene signature and tumor mutational burden, and conversely a lower score of transforming growth factor-beta (TGF-β) gene signature particularly in immune excluded tumors, were associated with response to the anti-PD-L1 agent atezolizumab. These results suggest that there is likely significant heterogeneity in TILs beyond canonical cytotoxic and exhausted phenotypes. Detailed characterization of the T lymphocytes in the tumor and periphery is needed for precisely mapping the cells responsible for tumor recognition and control and defining predictive markers of response to CPI in bladder cancer.

As described in greater detail in the Examples section, experiments were designed to address the above points by interrogating the tumor microenvironment from patients with localized muscle-invasive bladder TCC, who either received or did not receive neoadjuvant anti-PD-L1 immunotherapy (atezolizumab, Roche/Genentech) prior to surgical resection. Droplet single-cell RNA-sequencing (dscRNA-seq) of >30,000 CD4⁺ and CD8⁺ T cells from paired tumor and adjacent non-malignant tissues reveals known CD4⁺ populations such as regulatory T cells, but also several novel populations of cytotoxic CD4⁺s. Cytotoxic CD4⁺ are also found in the peripheral blood of the same patients based on dscRNAseq of >120,000 circulating CD4⁺ and CD8⁺ cells before and after anti-PD-L1 treatment. Paired TCR sequencing from the same cells revealed that the cytotoxic CD4⁺ populations are clonally expanded in the tumor microenvironment indicative of tumor specificity. This was validated by the direct autologous killing of tumor cells by cytotoxic CD4⁺ cells ex vivo. Although cytotoxic CD4⁺ display extensive phenotypic diversity, specific subsets of blood and tumor-infiltrating cytotoxic CD4⁺ expressing GZMB, NKG7, and GNLY are clonally and functionally related based on expression and TCR sequence, indicating that these particular effectors participate in systemic responses both in the circulation as well as tissue. Furthermore, we find that patients who clinically responded expand their circulating cytotoxic CD4⁺ T cells following anti-PD-L1 treatment. Overall these findings highlight the importance of CD4⁺ cytotoxic T cell effector populations for systemic tumor control and responses in patients after immunotherapy.

As will be discussed in more detail below, the disclosure provides novel population of CD4⁺ T cells specific to the bladder tumor microenvironment that express markers (e.g., proteins) indicative of cytolytic capabilities. As demonstrated below, this novel cytolytic CD4⁺ T cell population could be cultured ex vivo to form an expanded population of CD4⁺ T cells with enhanced cytolytic capabilities. In addition, experimental data presented below further demonstrate that an ex vivo population of cytolytic CD4⁺ T cells is capable of killing bladder cancer tumor cells (e.g., autologous bladder cancer tumor cells). Furthermore, a subset of these cytolytic CD4⁺ with the same function and antigenic specificity circulate in peripheral blood of individuals having bladder cancer and could be amenable to isolation from the blood of patients with bladder cancer.

### I. General Techniques

The practice of the claimed subject matter employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

### II. Definitions

Unless otherwise defined, all terms (including technical and scientific terms) used herein have the same meaning as commonly understood by one of ordinary skill in the art to which the claimed subject matter belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used in this specification and the appended claims, the term “or” is generally employed in its sense including “and/or” unless the content clearly dictates otherwise.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, an “individual” and “subject” are used interchangeably to refer to any subject to be treated. The subject can include animals and in particular mammals, such as human (e.g., human subjects) and non-human animals. Thus, the subject can be a human patient or an individual who has or is suspected of having a condition of interest (e.g., cancer) and/or one or more symptoms of the condition. The subject can also be an individual who is diagnosed with a risk of the condition of interest at the time of diagnosis or later. The term “non-human animals” includes all vertebrates, e.g., birds, e.g., mammals, e.g., rodents, e.g., mice, such as non-human primates (e.g., simians), e.g., sheep, dogs, cats, horses, cows, etc.

As used herein, the terms “treat,” treating,” “treatment,” and the like refer to reducing or ameliorating a disorder and/or symptoms associated therewith. When referring to a disease or condition, the term “treatment” is used herein to mean that at least an amelioration of the symptoms associated with the condition afflicting an individual is achieved, where amelioration is used in a broad sense to refer to at least a reduction in the magnitude of a parameter, e.g., a symptom, associated with the disease or condition (e.g., cancer) being treated. As such, treatment also includes situations where the pathological condition, or at least symptoms associated therewith, are completely inhibited, e.g., prevented from happening, or eliminated entirely such that the host no longer suffers from the condition, or at least the symptoms that characterize the condition. Thus, treatment includes: (i) prevention, that is, reducing the risk of development of clinical symptoms, including causing the clinical symptoms not to develop, e.g., preventing disease progression; (ii) inhibition, that is, arresting the development or further development of clinical symptoms, e.g., mitigating or completely inhibiting an active disease. It will be appreciated that, although not precluded, treating a disorder or condition does not require that the disorder, condition or symptoms associated therewith be completely eliminated.

“Pharmaceutically acceptable excipient” and “pharmaceutically acceptable carrier” refer to any suitable substance that provides a pharmaceutically acceptable carrier, additive or diluent for administration of a compound(s) of interest to a subject. Examples of pharmaceutically acceptable excipients include substances that aid the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethylcellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

Although various features of the disclosure may be described in the context of a single embodiment, the features may also be provided separately or in any suitable combination. Conversely, although the disclosure may be described herein in the context of separate embodiments for clarity, the disclosure may also be implemented in a single embodiment. Any published patent applications and any other published references, documents, manuscripts, and scientific literature cited herein are incorporated herein by reference for any purpose. In addition, the materials, methods, and examples are illustrative only and not intended to be limiting.

Reference in the specification to “embodiments,” “certain some embodiments,” “some embodiments,” “an embodiment,” “one embodiment” or “other embodiments” means that a particular feature, structure, or characteristic described in connection with those embodiments is included in at least some embodiments, but not necessarily all embodiments, of the disclosure.

It is understood that aspects and embodiments of the disclosure described herein include “comprising,” “consisting,” and “consisting essentially of” aspects and embodiments.

Headings, e.g., (a), (b), (i) etc., are presented merely for ease of reading the specification and claims. The use of headings in the specification or claims does not require the steps or elements be performed in alphabetical or numerical order or the order in which they are presented.

### III. Compositions of and Methods of Making Cytolytic CD4⁺ T Cells

The compositions disclosed herein include an ex vivo population of cytotoxic CD4⁺ T cells. As described in greater detail in the Examples section, a novel population of tumor-infiltrating CD4⁺ T lymphocytes has been identified specifically in bladder cancer tumor tissue. The novel CD4⁺ T cells displayed markers (e.g., proteins, peptides), identified at the RNA or protein level using techniques well known in the art (e.g., single cell RNAseq, ELISA, Western blotting, etc.) which allow them to be isolated (e.g., selected) from a heterogeneous population of cells. For example, the novel CD4⁺ population displayed one or a combination of markers including granzyme A (GZMA); granzyme B (GZMB); granzyme H (GZMH); granzyme K (GMZK); killer cell lectin-like receptor subfamily B, member 1 (KLRB1); killer cell lectin-like receptor subfamily D, member 1 (KLRD1); granulysin (GNLY); natural killer cell granule protein 7 (NKG7); chemokine (c-c motif) ligand 4 (CCL4); chemokine (c-c motif) ligand 5 (CCL5); lymphotoxin-beta (LTB); C-X-C chemokine receptor type 4 (CXCR4); C-X-C chemokine receptor type 6 (CXCR6); killer cell lectin-like receptor subfamily G, member 1 (KLRG1); perforin (PRF1); lymphocyte-activating gene 3 (LAG3); and chemokine (C-X-C motif) ligand 13 (CXCL13). These markers are associated with the ability to induce programmed cell death in target cells, recruit cells capable of inducing cell death, and/or are generally associated with cytotoxic cells (e.g., cytotoxic T cells (e.g., CD8⁺ T cells) and/or natural killer (NK) cells). As described in greater detail in the Examples below, cytotoxic CD4⁺ T cells expressing these markers and identical antigenic specificity were also found to circulate in the blood of the same bladder cancer patients which allow them to be isolated from the peripheral blood derived from patients with bladder cancer.

Granzymes are a family of serine proteases that can be released from cytoplasmic granules located within cytotoxic T cells and NK cells. The release of the granzyme can induce programmed cell death in target cells, thereby eliminating cells that have, for example, become cancerous or been infected with viruses or bacteria. Granzymes are generally packaged into cytotoxic granules with perforin, a pore-forming cytolytic protein which facilitates entry of the granzyme into the cytosol of the target cell. Five granzymes are encoded by the human genome: GZMA, GZMB, GZMH, GZMK, and GZMM. Granzymes are capable of inducing apoptosis through caspase-dependent and independent mechanisms.

KLRD1 and KLRB1 proteins are generally expressed in NK cells. KLDR1 is an antigen preferentially expressed on NK cells and is classified as a type II membrane protein because it has an external C-terminus. KLRB1 is also classified as a type II membrane protein due to the presence of an external C-terminus. Both KLRD1 and KLRB1 may be involved in the regulation of NK cell function.

Granulysin is a substance released by cytotoxic T cells, for example CD8⁺ T cells, and NK cells upon interaction (e.g., binding) with target cells. Granulysin generates holes in the target cell membrane resulting in apoptosis. Similar to granzymes, granulysin is generally packaged into granules with perforin, and may further be packed with granzymes.

NKG7 is expressed in both activated NK cells and T cells and is a granule-associated protein.

CCL4 is crucial for immune responses towards infection and inflammation, and functions as a chemoattractant for NK cells, among a variety of other immune cells. CCL5 also acts as a chemokine for T cells, eosinophils, and basophils, and plays an important role in recruiting leukocytes to sites of inflammation. Further, CCL5 may act to induce proliferation and activation of NK cells.

LTB, also known as tumor necrosis factor C (TNF-C), is a type II membrane protein of the TNF family that functions to induce an inflammatory response. LTB is expressed on activated lymphocytes.

CXCR4 is an alpha-chemokine receptor specific for CXCL12, which is a molecule with potent chemotactic activity for lymphocytes. CXCR6 is expressed on different types of T cells (e.g., CD8⁺ T cells) and becomes upregulated following T cell activation.

CXCL13 is a small cytokine that belongs to the CXC family of chemokines and may be expressed on T lymphocytes. CXCL13 interacts with chemokine receptor CXCR5, thereby recruiting B cells of the B-1 and B-2 subsets.

Thus, in one aspect, provided herein is an ex vivo population of CD4⁺ T cells wherein the CD4⁺ T cells express one or more markers selected from the group consisting of GZMA, GZMB, GZMH, GZMK, KLRB1, KLRD1, GNLY, NKG7, CCL4, CCL5, LTB, CXCR4, CXCR6, PRF1, KLRG1, LAG3, CXCL13, and combinations of any thereof, and wherein the CD4⁺ T cells have cytolytic capabilities. In some embodiments, the CD4⁺ cells of the ex vivo CD4⁺ population express one or a combination of markers selected from the group consisting of GZMA, GZMB, GZMH, GMZK, KLRB1, KLRD1, GNLY, NKG7, CCL4, CCL5, LTB, CXCR4, CXCR6, PRF1, KLRG1, LAG3, and CXCL13, and combinations of any thereof. In some embodiments, the CD4⁺ cells of the ex vivo CD4⁺ population of the disclosure express the marker LAG3.

In some embodiments, in addition to the markers described above, the CD4⁺ cells of the CD4⁺ populations of the disclosure can be found to not express one or more additional immune checkpoint markers. An “immune checkpoint marker” or “checkpoint marker” as referred to herein is used in the usual and customary sense to describe molecules that regulate the immune system (e.g., immune system response). Checkpoint markers (e.g., inhibitory checkpoint markers that result in immunosuppression) can be targets for cancer immunotherapy. Non-limiting examples of immune checkpoint markers include CLTA-4, PD-1, and PD-L1. Other examples of immune checkpoint markers include, but are not limited to IL2RA/CD25, TNFRSF4/OX40, TNFRSF9/4-1BB, TNFRSF18/GITR, CD278/ICOS, and TIGIT.

Accordingly, in some embodiments, the cytolytic CD4⁺ cells of the CD4⁺ populations do not express one or more additional immune checkpoint markers. In some embodiments, the one of more additional immune checkpoint markers which are not expressed by the cytolytic CD4⁺ cells are selected from the group consisting of IL2RA/CD25, TNFRSF4/OX40, TNFRSF9/4-1BB, TNFRSF18/GITR, CD278/ICOS, and TIGIT. In some embodiments, the cytolytic CD4⁺ cells of the ex vivo populations of the disclosure express LAG3 but lack expression of one or more additional immune checkpoint markers.

The term “lack”, or “lacking” or “lack of” when used in reference to the expression of an immune checkpoint marker as described herein, refers to a level of the immune checkpoint expression which is undetectable by the methods as used herein to measure such levels. The term “lack” generally refers to minimal, absent, or about null levels of the immune checkpoint expression, but does not necessarily mean that the immune checkpoint is completely absent. In some embodiments, the phrase “lack expression of an immune checkpoint” refers to the level of the immune checkpoint expression in a cell which is below a detectable level in that cell. In some embodiments, the term “lack” refers to the level of a given immune checkpoint expression in a cell which is below a detectable level in that cell as determined by FACS analysis. In some embodiments, when referring to a population of CD4⁺ cells, the phrase “lack of expression” refers to a CD4⁺ cell population of less than 25%, less than 20%, less than 15%, less than 10%, less than 5%, or less than 1% of CD4⁺ cells expressing a given immune checkpoint marker.

In some embodiments, the cytolytic CD4⁺ cells of the ex vivo populations of the disclosure express LAG3 but lack expression of one or more additional immune checkpoint markers selected from the group consisting of IL2RA/CD25, TNFRSF4/OX40, TNFRSF9/4-1BB, TNFRSF18/GITR, CD278/ICOS, TIGIT, and combinations of any thereof. In some embodiments, the cytolytic CD4⁺ cells of the ex vivo populations of the disclosure express LAG3 but do not express one or more additional immune checkpoint markers at a detectable level, as determined by FACS analysis. In some embodiments, the ex vivo population of cytolytic CD4⁺ cells includes less than 25%, less than 20%, less than 15%, less than 10%, less than 5%, or less than 1% of CD4⁺ cells expressing LAG3 but do not express one or more additional immune checkpoint markers at a detectable level. In some embodiments, the one or more additional immune checkpoint markers not expressed by the CD4⁺ cells is selected from the group consisting of IL2RA/CD25, TNFRSF4/OX40, TNFRSF9/4-1BB, TNFRSF18/GITR, CD278/ICOS, TIGIT, and combinations of any thereof.

Further analysis of the novel CD4⁺ population specific to the bladder tumor microenvironment revealed selective enrichment of heat shock proteins and IFN-gamma (also referred to herein as IFNG or IFNγ). Heat shock proteins can be expressed in response to conditions such as stress, exposure to cold, UV light, and during wound healing and tissue remodeling. IFN-gamma is generally secreted by T helper cells (e.g., Th1 cells), cytotoxic T cells (e.g., CD8⁺ T cells), macrophages, mucosal epithelial cells, and NK cells. IFN-gamma has been suggested to promote NK activation, increase antigen presentation and lysosome activity of macrophages, and activate inducible nitric oxide synthase, among other activities. Thus, in some embodiments, the ex vivo population of CD4⁺ cells also express heat shock proteins and/or IFN-gamma. In some embodiments, the CD4⁺ cells express heat shock proteins. In some embodiments, the CD4⁺ cells express IFN-gamma.

In some embodiments, provided herein is an ex vivo population of cytolytic CD4⁺ T cells. To determine cytolytic capability (e.g., the ability to induce cell death in target cells), various methods known in the art may be used. Non-limiting examples of methods suitable for determination of cytolytic capability of the cytolytic CD4 cells disclosed herein include lysis of autologous tumor cells (e.g., primary or transformed cells), allogeneic human tumor cells, or mouse mastocytoma target cells (P815) loaded with anti-human CD3 antibodies. Accordingly, in some embodiments, cytolytic capabilities of the cytolytic CD4⁺ cells disclosed herein are detected by lysis of autologous tumor cells (e.g., primary or transformed cells). In some embodiments, cytolytic capabilities of the cytolytic CD4⁺ cells disclosed herein are detected by allogeneic human tumor cells. In some embodiments, cytolytic capabilities of the cytolytic CD4⁺ cells disclosed herein are detected by lysis of mouse mastocytoma target cells (P815) loaded with anti-human CD3 antibodies. In some embodiments, cytolytic capabilities of the cytolytic CD4⁺ cells disclosed herein are detected by lysis of autologous tumor cells (e.g., primary or transformed cells). In some embodiments, cytolytic capabilities are detected by allogeneic human tumor cells. In a further embodiment, cytolytic capabilities are detected by lysis of mouse mastocytoma target cells (P815) loaded with anti-human CD3 antibodies. In some embodiments, a TUNEL assay can be used to determine cytotoxic capabilities. In some embodiments, markers for caspases may be used to determine cytotoxic capabilities. In some embodiments, cytolytic capabilities are detected as described in Example 15.

In order to promote apoptosis of target cells, T cells specifically interact with (e.g., bind) target cells. Activated T cells use T cell receptors (TCRs) to identify antigens on the surface of cells. In this way, activated T cells specifically target cell populations. In humans, TCRs in 95% of T cells are composed of an alpha and a beta chain. The alpha and beta chains are encoded by TRA and TRB genes, respectively. Alpha and beta chains are expressed as part of a complex with CD3 chain molecules. The variable domain of both the TCR alpha chain and beta chain contain three complementarity-determining regions (CDRs): CDR1, CDR2, and CDR3. CDR3 of both the alpha and beta chain serves as the main CDR responsible for antigen recognition. It will be appreciated by those skilled in the art that TCRs of CD4⁺ T cells recognize antigens bound to MHC II molecules.

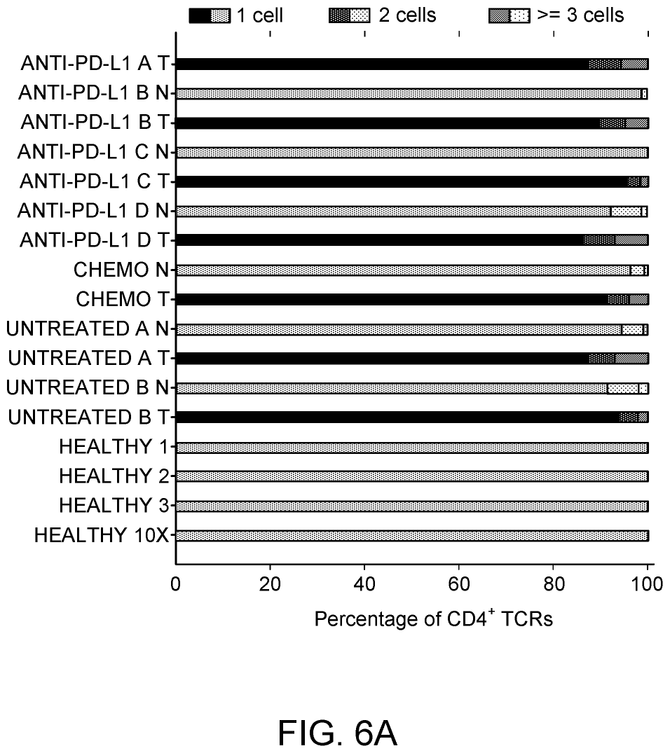

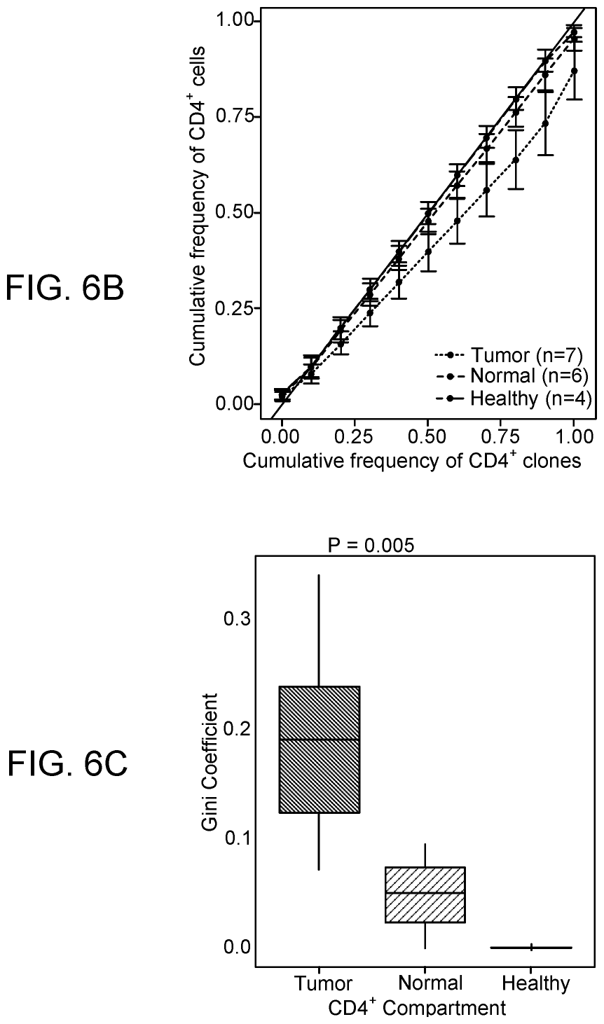

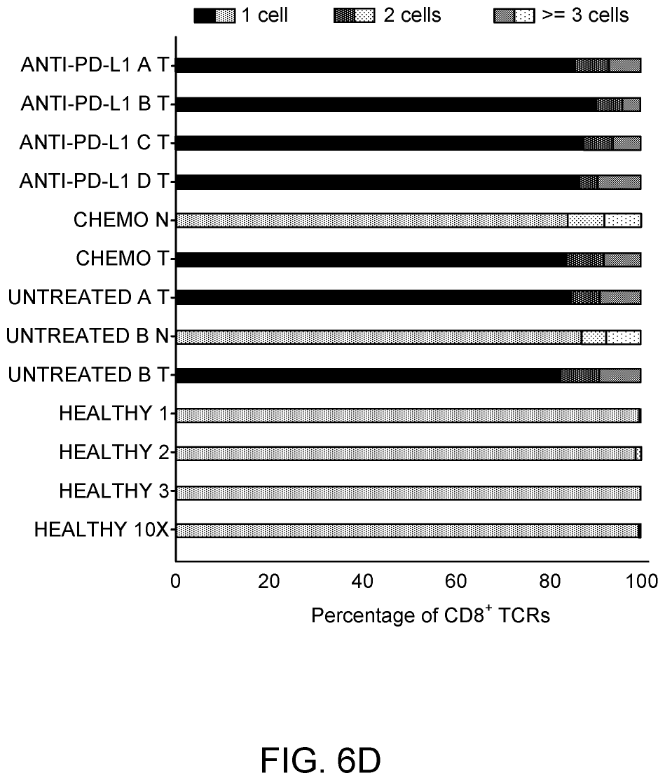

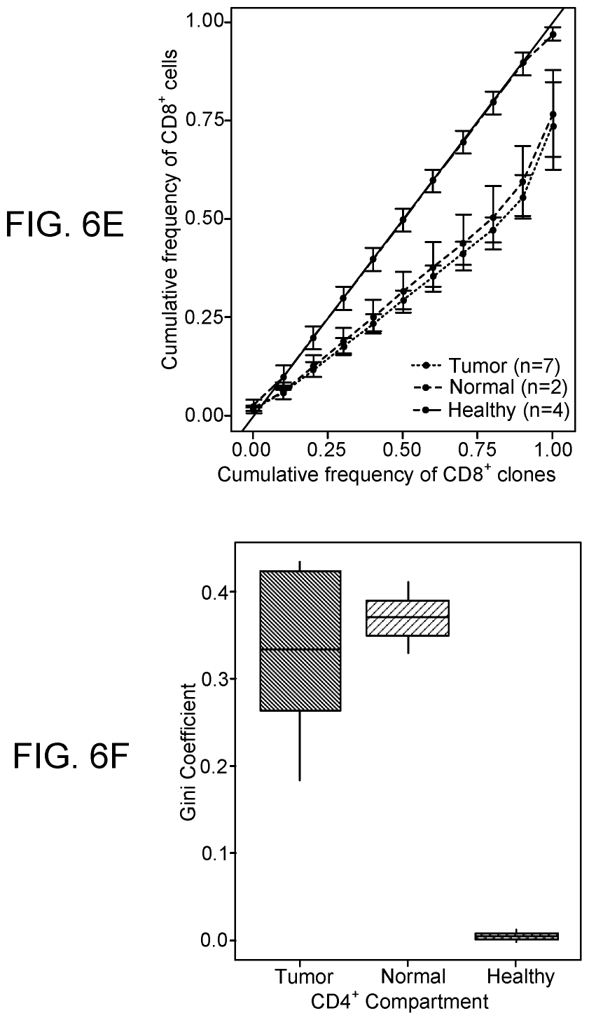

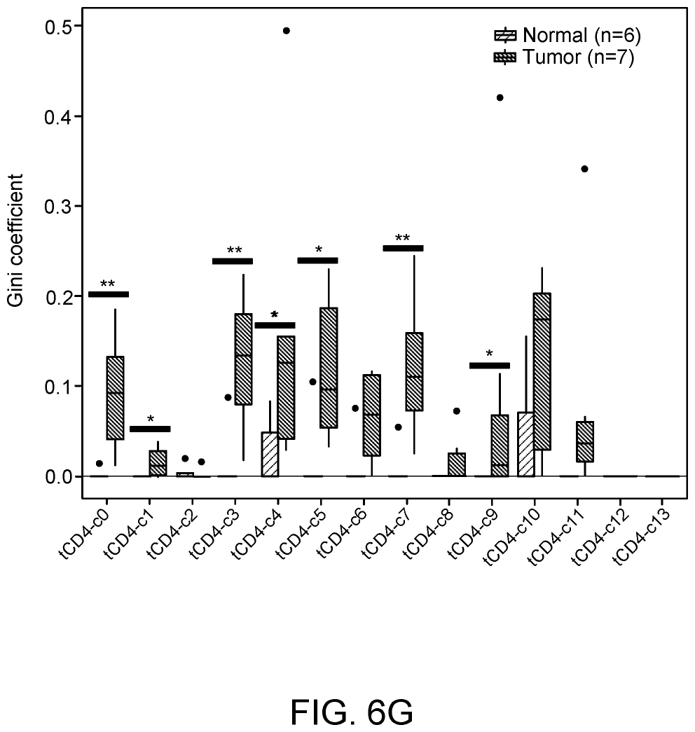

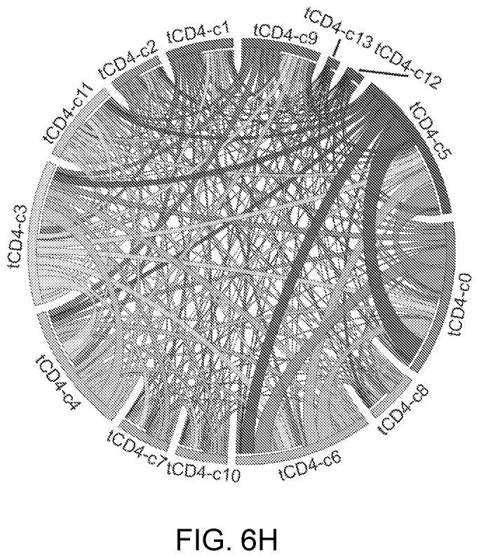

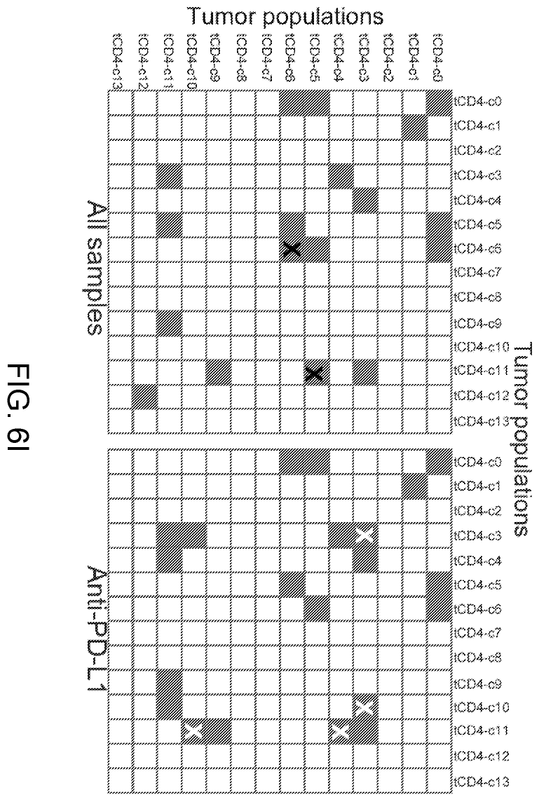

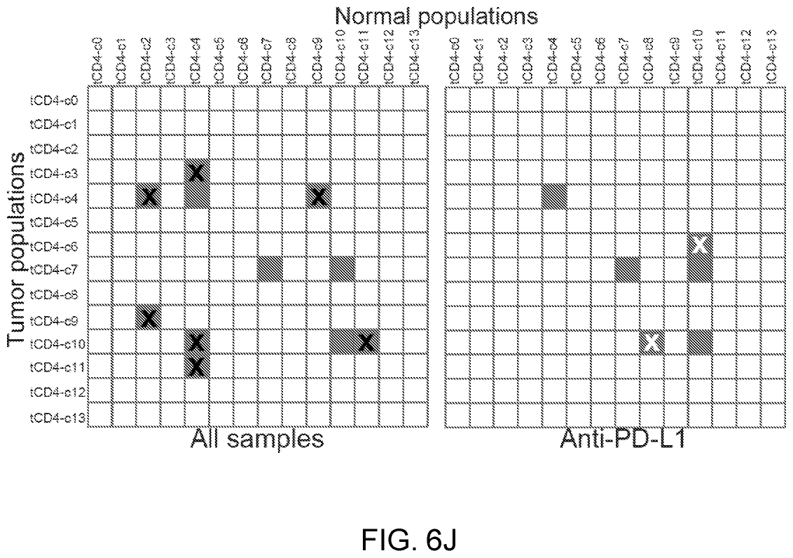

The identified novel CD4⁺ T cells specific to the bladder tumor microenvironment were found to share expression of specific CDR3 amino acid sequences for both the alpha and beta chains (see, e.g., Example 8 and FIG. 6G), suggesting that the novel population may target bladder cancer tumor specific antigens. Thus, in some embodiments, the CD4⁺ cells of the ex vivo population of CD4⁺ cells share the same alpha chain CDR3 amino acid sequence. In some embodiments, the CD4⁺ cells of the ex vivo population of CD4⁺ cells share the same beta chain CDR3 amino acid sequence. In some embodiments, the CD4⁺ cells of the ex vivo population of CD4⁺ cells share the same alpha chain and beta chain CDR3 amino acid sequences. In some embodiments, the CD4⁺ cells express T cell receptor (TCR) that includes: (i) a TCR alpha CDR3 sequence selected from the group consisting of CALRGTNTGNQFYF (SEQ ID NO: 1), CAVEAGGVVGSARQLTF (SEQ ID NO: 2), CAIIIQGAQKLVF (SEQ ID NO:3), CAFMKGNNDMRF (SEQ ID NO:4), CATADKGGAQKLVF (SEQ ID NO:5), CVVNGGGDKLIF (SEQ ID NO:6), CATENLQGAQKLVF (SEQ ID NO:7), CAPPRRVDSDGQKLLF (SEQ ID NO:8), and CALNNAGNMLTF (SEQ ID NO:9); and (ii) a TCR beta CDR3 sequence seleted from the group consisting of CASSSAMVAGAYEQYF (SEQ ID NO: 10), CSARDPKGGDTEAFF (SEQ ID NO: 11), CASSQSRDRTYEQYF (SEQ ID NO: 12), CASSQGANQPQHF (SEQ ID NO: 13), CASSSILGEVATGELFF (SEQ ID NO: 14), CASSRTGGTEAFF (SEQ ID NO: 15), CASSQDPTYEQYF (SEQ ID NO: 16), CASSPTGTGIRGYTF (SEQ ID NO: 17), CASSLIPGQGATSYGYTF (SEQ ID NO: 18), and CASSLARSFGLGEQFF (SEQ ID NO: 19). In some embodiments, the CD4⁺ cells express T cell receptor (TCR) including a TCR alpha CDR3 sequence selected from the group consisting of CALRGTNTGNQFYF (SEQ ID NO: 1), CAVEAGGVVGSARQLTF (SEQ ID NO: 2), CAIIIQGAQKLVF (SEQ ID NO:3), CAFMKGNNDMRF (SEQ ID NO:4), CATADKGGAQKLVF (SEQ ID NO:5), CVVNGGGDKLIF (SEQ ID NO:6), CATENLQGAQKLVF (SEQ ID NO:7), CAPPRRVDSDGQKLLF (SEQ ID NO:8), and CALNNAGNMLTF (SEQ ID NO:9). In some embodiments, the CD4⁺ cells express T cell receptor (TCR) including a TCR beta CDR3 sequence seleted from the group consisting of CASSSAMVAGAYEQYF (SEQ ID NO: 10), CSARDPKGGDTEAFF (SEQ ID NO: 11), CASSQSRDRTYEQYF (SEQ ID NO: 12), CASSQGANQPQHF (SEQ ID NO: 13), CASSSILGEVATGELFF (SEQ ID NO: 14), CASSRTGGTEAFF (SEQ ID NO: 15), CASSQDPTYEQYF (SEQ ID NO: 16), CASSPTGTGIRGYTF (SEQ ID NO: 17), CASSLIPGQGATSYGYTF (SEQ ID NO: 18), and CASSLARSFGLGEQFF (SEQ ID NO: 19).

The ex vivo population of CD4⁺ T cells as described herein, including embodiments thereof, may be obtained from a biological sample. The term “biological sample” as used herein refers to materials obtained from or derived from an individual, a subject, or a patient. A biological sample includes sections of tissues, such as biopsy (e.g., tumor biopsy) and autopsy samples, resected tissues (e.g., resected tumors), and frozen sections taken for histological purposes. Such samples include bodily fluids such as blood and blood fractions or products (e.g., serum, plasma, platelets, red blood cells, circulating tumor cells, and the like), lymph, sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. As the novel cytolytic CD4⁺ T cell population was found to be specific to the bladder tumor environment, it is contemplated that the biological sample may be obtained from an individual with a bladder cancer tumor. Thus, in some embodiments, the biological sample is a bladder cancer tumor. In some embodiments, the bladder cancer tumor is obtained via resection. In some embodiments, the bladder cancer tumor is obtained via tumor biopsy. The term “tumor biopsy” refers to tumor tissue sample taken by appropriate means, such as via fine needle biopsy, core needle biopsy, excisional or incisional biopsy, endoscopic biopsy, laparascopic biopsy, thorascopic mediastrinoscopic biopsy, laparotomy, thoracotomy, skin biopsy, and sentinel lymph node mapping and biopsy. Any suitable method for obtaining a tissue sample of a tumor may be used in conjunction with the methods as provided herein.

Accordingly, in some embodiments, the CD4⁺ T cells are obtained from a biological sample including bladder cancer cells. In some embodiments, the CD4⁺ T cells are obtained from a biological sample including peripheral blood from an individual having or suspected of having bladder cancer. In some embodiments, the bladder cancer is squamous cell carcinoma. In some embodiments, the bladder cancer is non-squamous cell carcinoma. In some embodiments, the bladder cancer is adenocarcinoma. In some embodiments, the bladder cancer is small cell carcinoma. In some embodiments, the bladder cancer is early stage bladder cancer, non-metastatic bladder cancer, non-invasive bladder cancer, non-muscle-invasive bladder cancer, primary bladder cancer, advanced bladder cancer, locally advanced bladder cancer (such as, e.g., unresectable locally advanced bladder cancer), metastatic bladder cancer, bladder cancer in remission, progressive bladder cancer, or recurrent bladder cancer. In some embodiments, the bladder cancer is localized resectable, localized unresectable, or unresectable. In some embodiments, the bladder cancer is a high grade, non-muscle-invasive cancer that has been refractory to standard intra-bladder infusion (intravesical) therapy.

Despite the presence of the novel CD4⁺ T cell population including cytolytic markers (e.g., granzymes) at the tumor site, the inability of the potentially cytolytic CD4⁺ T cells to prevent tumor growth suggests that the cytolytic ability of the native CD4⁺ population may be impaired in vivo. As seen in the Examples, however, culturing and expansion of the novel CD4⁺ population resulted in successful killing of bladder cancer cells (see FIGS. 6A-6C). Therefore, in some embodiments, the CD4⁺ T cells have decreased cytolytic capabilities as compared to the CD4⁺ T cells which have been expanded ex vivo. In some embodiments, the CD4⁺ T cells have decreased cytolytic capabilities, e.g., have a reduction by a statistically significant amount of cytolytic capabilities compared to the CD4⁺ T cells which have been expanded ex vivo. In some embodiments, the CD4⁺ T cells having decreased cytolytic capabilities can be CD4⁺ T cells having a reduction in their cytolytic capabilities by at least about 5% as compared to the cytolytic capabilities of a reference population of CD4⁺ T cells, for example a decrease by at least about 5%, at least about 10%, at least about 20%, at least about 30%, at least about 40%, at least about 50%, at least about 60%, at least about 70%, at least about 80%, at least about 90%, at least about 95%, at least about 99% up to and including 100% decrease (i.e. null level as compared to a reference population of CD4⁺ T cells), or any integer in between 5% and 100% as compared to a reference population of CD4⁺ T cells, e.g., a population of CD4⁺ T cells which have been expanded ex vivo.

It is contemplated that the ex vivo population can be enriched for the novel cytolytic T cell population. The terms “enriching” or “enriched” are used interchangeably herein and mean that the fraction of cells of one cell type (cytolytic CD4⁺ T cells) is increased by at least 40% over the fraction of cells of that type in the starting culture or preparation. In some embodiments, the population is at least 50%, 60%, 70%, 80%, 90%, or 95% enriched for cytolytic CD4⁺ T cells. In some embodiments, the population is at least 50% enriched for cytolytic CD4⁺ T cells. In some embodiments, the population is at least 55% enriched for cytolytic CD4⁺ T cells. In some embodiments, the population is at least 60% enriched for cytolytic CD4⁺ T cells. In some embodiments, the population is at least 65% enriched for cytolytic CD4⁺ T cells. In some embodiments, the population is at least 70% enriched for cytolytic CD4⁺ T cells. In some embodiments, the population is at least 75% enriched for cytolytic CD4⁺ T cells. In some embodiments, the population is at least 80% enriched for cytolytic CD4⁺ T cells. In some embodiments, the population is at least 81% enriched for cytolytic CD4⁺ T cells. In some embodiments, the population is at least 82% enriched for cytolytic CD4⁺ T cells. In some embodiments, the population is at least 83% enriched for cytolytic CD4⁺ T cells. In some embodiments, the population is at least 84% enriched for cytolytic CD4⁺ T cells. In some embodiments, the population is at least 85% enriched for cytolytic CD4⁺ T cells. In some embodiments, the population is at least 86% enriched for cytolytic CD4⁺ T cells. In some embodiments, the population is at least 87% enriched for cytolytic CD4⁺ T cells. In some embodiments, the population is at least 88% enriched for cytolytic CD4⁺ T cells. In some embodiments, the population is at least 89% enriched for cytolytic CD4⁺ T cells. In some embodiments, the population is at least 90% enriched for cytolytic CD4⁺ T cells. In some embodiments, the population is at least 91% enriched for cytolytic CD4⁺ T cells. In some embodiments, the population is at least 92% enriched for cytolytic CD4⁺ T cells. In some embodiments, the population is at least 93% enriched for cytolytic CD4⁺ T cells. In some embodiments, the population is at least 94% enriched for cytolytic CD4⁺ T cells. In some embodiments, the population is at least 95% enriched for cytolytic CD4⁺ T cells. In some embodiments, the population is at least 96% enriched for cytolytic CD4⁺ T cells. In some embodiments, the population is at least 97% enriched for cytolytic CD4⁺ T cells. In some embodiments, the population is at least 98% enriched for cytolytic CD4⁺ T cells. In some embodiments, the population is at least 99% enriched for cytolytic CD4⁺ T cells. In some embodiments, the population is 100% enriched for cytolytic CD4⁺ T cells.

Given the compositions provided herein may be enriched for cytolytic CD4⁺ T cells specific to the bladder cancer tumor environment, the compositions described herein will be endowed with the ability to kill bladder cancer cells. In some embodiments, the CD4⁺ T cells are capable of killing autologous cancer cells. In some embodiments, the CD4⁺ T cells are capable of killing autologous primary cancer cells. In further embodiments, the CD4⁺ T cells are capable of killing autologous transformed cancer cells. In some embodiments, the CD4⁺ T cells are capable of killing allogeneic cancer cells.

### Pharmaceutical Compositions

The ex vivo CD4⁺ populations of the disclosure can be incorporated into compositions, including pharmaceutical compositions. Such compositions generally can include the ex vivo CD4⁺ cell populations and a pharmaceutically acceptable excipient. Accordingly, in one aspect, some embodiments of the disclosure relate to pharmaceutical compositions including a population of CD4⁺ T cells with cytolytic capabilities and a pharmaceutically acceptable excipient. In some embodiments, the CD4⁺ cell population is at least 50%, 60%, 70%, 80%, 90%, or 95% enriched for CD4⁺ T cells with cytolytic capabilities. In some embodiments, the CD4⁺ cell population is at least 50% enriched for CD4⁺ T cells with cytolytic capabilities. In some embodiments, the CD4⁺ cell population is at least 55% enriched for CD4⁺ T cells with cytolytic capabilities. In some embodiments, the CD4⁺ cell population is at least 60% enriched for CD4⁺ T cells with cytolytic capabilities. In some embodiments, the population is at least 65% enriched for CD4⁺ T cells with cytolytic capabilities. In some embodiments, the CD4⁺ cell population is at least 70% enriched for CD4⁺ T cells with cytolytic capabilities. In some embodiments, the CD4⁺ cell population is at least 75% enriched for CD4⁺ T cells with cytolytic capabilities. In some embodiments, the CD4⁺ cell population is at least 80% enriched for CD4⁺ T cells with cytolytic capabilities. In some embodiments, the CD4⁺ cell population is at least 81% enriched for CD4⁺ T cells with cytolytic capabilities. In some embodiments, the population is at least 82% enriched for CD4⁺ T cells with cytolytic capabilities. In some embodiments, the CD4⁺ cell population is at least 83% enriched for CD4⁺ T cells with cytolytic capabilities. In some embodiments, the CD4⁺ cell population is at least 84% enriched for CD4⁺ T cells with cytolytic capabilities. In some embodiments, the CD4⁺ cell population is at least 85% enriched for CD4⁺ T cells with cytolytic capabilities. In some embodiments, the CD4⁺ cell population is at least 86% enriched for CD4⁺ T cells with cytolytic capabilities. In some embodiments, the CD4⁺ cell population is at least 87% enriched for CD4⁺ T cells with cytolytic capabilities. In some embodiments, the CD4⁺ cell population is at least 88% enriched for CD4⁺ T cells with cytolytic capabilities. In some embodiments, the CD4⁺ cell population is at least 89% enriched for CD4⁺ T cells with cytolytic capabilities. In some embodiments, the CD4⁺ cell population is at least 90% enriched for CD4⁺ T cells with cytolytic capabilities. In some embodiments, the CD4⁺ cell population is at least 91% enriched for CD4⁺ T cells with cytolytic capabilities. In some embodiments, the CD4⁺ cell population is at least 92% enriched for CD4⁺ T cells with cytolytic capabilities. In some embodiments, the CD4⁺ cell population is at least 93% enriched for CD4⁺ T cells with cytolytic capabilities. In some embodiments, the CD4⁺ cell population is at least 94% enriched for CD4⁺ T cells with cytolytic capabilities. In some embodiments, the CD4⁺ cell population is at least 95% enriched for CD4⁺ T cells with cytolytic capabilities. In some embodiments, the CD4⁺ cell population is at least 96% enriched for CD4⁺ T cells with cytolytic capabilities. In some embodiments, the CD4⁺ cell population is at least 97% enriched for CD4⁺ T cells with cytolytic capabilities. In some embodiments, the CD4⁺ cell population is at least 98% enriched for CD4⁺ T cells with cytolytic capabilities. In some embodiments, the CD4⁺ cell population is at least 99% enriched for CD4⁺ T cells with cytolytic capabilities. In some embodiments, the CD4⁺ cell population is 100% enriched for CD4⁺ T cells with cytolytic capabilities.

In some embodiments, the pharmaceutical composition of the disclosure can be formulated to be compatible with its intended route of administration. The cytolytic CD4⁺ T cell population disclosed herein may be administered through a parenteral route. Examples of parenteral routes of administration include, for example, intramuscular, intravenous, intradermal, subcutaneous, transdermal (topical), transmucosal, intra-peritoneal and intraomental administration. Solutions or suspensions used for parenteral application can include the following components: a sterile diluent such as water for injection, saline solution, tissue preservation solution, heparin containing isotonic fluid (Plasma-LyteA, normal saline), CMRL 1066, +50 mL 25% human serum albumin containing heparin, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5). Agents that increases viscosity, such as sodium carboxymethyl cellulose, sorbitol, dextran, hydrogel, or fibrin, may be included to facilitate cell aggregation. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. Suitable carriers include physiological saline, bacteriostatic water, Cremophor EL™. (BASF, Parsippany, N.J.) or phosphate buffered saline (PBS). In all cases, the composition should be sterile and should be fluid to the extent that easy syringability exists. It should be stable under the conditions of preparation and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. Prevention of the action of microorganisms can be achieved by various antibacterial and antifungal agents, for example, parabens, chlorobutanol, phenol, ascorbic acid, thimerosal, and the like. In many cases, isotonic agents can be included, for example, sugars, polyalcohols such as mannitol, sorbitol, sodium chloride in the composition.

Pharmaceutical formulations for parenteral administration include aqueous solutions of active compounds. For injection, the pharmaceutical compositions of the present disclosure may be formulated in aqueous solutions, preferably in physiologically compatible buffers such as Hank's solution, Ringer' solution, Wisconsin Solution, Plasma-LyteA, or physiologically buffered saline. Aqueous injection suspensions may contain substances which increase the viscosity of the suspension, such as sodium carboxymethyl cellulose, sorbitol, dextran, hydrogel, or fibrin. Human serum albumin may be included to support cell viability. Additionally, suspensions of the active solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

After the pharmaceutical compositions disclosed herein have been prepared and are formulated in an acceptable carrier, they can be placed in an appropriate container and labeled for treatment of an indicated condition with information including amount, frequency and method of administration. In some embodiments, any of the pharmaceutical compositions disclosed herein are loaded into a syringe (such as a pre-filled syringe), a catheter, or a cannula prior to administration into an individual.

The data obtained from the cell culture assays and animal studies can be used in formulating a range of dosage for use in humans. The dosage of such therapy can be estimated initially from preclinical data comparing the relative potency of the new formulation with the standard formulation in animal studies. A minimal cell dose to achieve reduction in bladder tumor size and/or increase survival time in animal studies can be compared between old and new formulations. In these dose-finding studies, tumor size at baseline and after administration of the pharmaceutical composition can be measured and compared between the old and new formulation. Such information can be used to more accurately determine useful doses in humans.

The dosages administered will vary from individual to individual; a therapeutically effective dose in humans can be estimated, for example but not limited to, by the change in tumor size. This dosage may be repeated as considered appropriate by the treating physician.

Also provided herein are methods for making (e.g., producing) an ex vivo expanded population of CD4⁺ T cells with cytolytic capabilities. The method includes (a) separating CD4⁺ T cells from a biological sample containing a mixture of different types of immune cells, (b) culturing the separated CD4⁺ T cells in media containing IL-2 in an amount sufficient to promote the expansion of CD4⁺ T cells, and (c) splitting the cultured CD4⁺ T cells to promote the enrichment of CD4⁺ T cells thus producing an ex vivo expanded population of CD4⁺ T cells with cytolytic capabilities.

Generally, the act of separating the CD4⁺ T cells of interest from the mixture of immune cells may be accomplished using any one of cell separation methods known in the art. For example, separating may be accomplished using one or more techniques selected from the group consisting of fluorescence activated cell sorting (FACs), magnetic activated cell sorting (MACs), buoyancy activated cell sorting (BACs), microfluidics, centrifugation, filtration, and combinations of any thereof. In some embodiments, the separation of the novel CD4⁺ T cells rely on the identification of one or more markers (e.g., GZMA, GZMB, CXCL13, IFNG) as described herein. As indicated supra, in some embodiments, the biological sample as described in step (a) of the production methods described herein can be a sample of a bladder cancer tumor. In some embodiments, the bladder cancer tumor is obtained via resection. In some embodiments, the bladder cancer tumor is obtained via tumor biopsy. In some embodiments, the biological sample includes bladder cancer cells. In some embodiments, the biological sample includes peripheral blood from an individual having or suspected of having bladder cancer.

In some embodiments, step (a) of the production methods described herein may include utilizing a sorting buffer. In some embodiments, the sorting buffer includes 500 ml of PBS, 2% heat inactivated FBS, 1 mM EDTA, and 1% penecillin streptomycin. In some embodiments, the buffer includes about 0.01% to about 3% heat inactivated FBS. In some embodiments, the buffer includes about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 mM EDTA.

In an embodiment, separating the CD4⁺ T cells from the biological sample, e.g., step (a), is performed as described in Example 13.

In general, the separated population of CD4⁺ T cells may be cultured using any one of suitable culturing techniques, systems, culture media, and strategies known in the art. In some embodiments, the culturing media contains T cell expansion medium (e.g., human T cell medium). In some embodiments, the culture media further includes about 1% to about 30% human AB serum. In some embodiments, the culture media further includes about 0.01% to about 5% antibiotics. In an embodiment, the antibiotic is penecillin streptomycin.

In some embodiments, the culture media includes human IL-2. In some embodiments, the culture media includes recombinant human IL-2. In some embodiments, IL-2 is used in the amount of about 1 IU/ml to about 2000 IU/ml. In some embodiments, IL-2 is used in the amount of about 10 IU/ml to about 2000 IU/ml. In some embodiments, IL-2 is used in the amount of 20 IU/ml to about 2000 IU/ml. In some embodiments, IL-2 is used in the amount of about 30 U/ml to about 2000 IU/ml. In some embodiments, IL-2 is used in the amount of about 40 U/ml to about 2000 IU/ml. In some embodiments, IL-2 is used in the amount of about 50 U/ml to about 2000 IU/ml. In some embodiments, IL-2 is used in the amount of about 60 U/ml to about 2000 IU/ml. In some embodiments, IL-2 is used in the amount of about 70 U/ml to about 2000 IU/ml. In some embodiments, IL-2 is used in the amount of about 80 U/ml to about 2000 IU/ml. In some embodiments, IL-2 is used in the amount of about 90 U/ml to about 2000 IU/ml. In some embodiments, IL-2 is used in the amount of 100 U/ml to about 2000 IU/ml. In some embodiments, IL-2 is used in the amount of about 150 U/ml to about 2000 IU/ml. In some embodiments, IL-2 is used in the amount of about 200 U/ml to about 2000 IU/ml. In some embodiments, IL-2 is used in the amount of about 250 U/ml to about 2000 IU/ml. In some embodiments, IL-2 is used in the amount of about 300 U/ml to about 2000 IU/ml. In some embodiments, IL-2 is used in the amount of about 350 U/ml to about 2000 IU/ml. In some embodiments, IL-2 is used in the amount of about 400 U/ml to about 2000 IU/ml. In some embodiments, IL-2 is used in the amount of about 450 U/ml to about 2000 IU/ml. In some embodiments, IL-2 is used in the amount of about 500 U/ml to about 2000 IU/ml. In some embodiments, IL-2 is used in the amount of about 550 U/ml to about 2000 IU/ml. In some embodiments, IL-2 is used in the amount of about 600 U/ml to about 2000 IU/ml. In some embodiments, IL-2 is used in the amount of about 650 U/ml to about 2000 IU/ml. In some embodiments, IL-2 is used in the amount of about 700 U/ml to about 2000 IU/ml. In some embodiments, IL-2 is used in the amount of about 750 U/ml to about 2000 IU/ml. In some embodiments, IL-2 is used in the amount of about 800 U/ml to about 2000 IU/ml. In some embodiments, IL-2 is used in the amount of about 850 U/ml to about 2000 IU/ml. In some embodiments, IL-2 is used in the amount of about 900 U/ml to about 2000 IU/ml. In some embodiments, IL-2 is used in the amount of about 950 U/ml to about 2000 IU/ml. In some embodiments, IL-2 is used in the amount of about 1000 U/ml to about 2000 IU/ml. In some embodiments, IL-2 is used in the amount of about 1100 U/ml to about 2000 IU/ml. In some embodiments, IL-2 is used in the amount of about 1150 U/ml to about 2000 IU/ml. In some embodiments, IL-2 is used in the amount of about 1200 U/ml to about 2000 IU/ml. In some embodiments, IL-2 is used in the amount of about 1250 U/ml to about 2000 IU/ml. In some embodiments, IL-2 is used in the amount of about 1300 U/ml to about 2000 IU/ml. In some embodiments, IL-2 is used in the amount of about 1350 U/ml to about 2000 IU/ml. In some embodiments, IL-2 is used in the amount of about 1400 U/ml to about 2000 IU/ml. In some embodiments, IL-2 is used in the amount of about 1450 U/ml to about 2000 IU/ml. In some embodiments, IL-2 is used in the amount of about 1500 to about 2000 IU/ml. In some embodiments, IL-2 is used in the amount of about 1550 U/ml to about 2000 IU/ml. In some embodiments, IL-2 is used in the amount of about 1600 to about 2000 IU/ml. In some embodiments, IL-2 is used in the amount of about 1650 U/ml to about 2000 IU/ml. In some embodiments, IL-2 is used in the amount of about 1700 U/ml to about 2000 IU/ml. In some embodiments, IL-2 is used in the amount of about 1750 U/ml to about 2000 IU/ml. In some embodiments, IL-2 is used in the amount of about 1800 U/ml to about 2000 IU/ml. In some embodiments, IL-2 is used in the amount of about 1850 to about 2000 IU/ml. In some embodiments, IL-2 is used in the amount of 1900 IU/ml to about 2000 IU/ml. In some embodiments, IL-2 is used in the amount of about 1950 U/ml to about 2000 IU/ml. In some embodiments, IL-2 is used in the amount of about 2000 U/ml to about 3000 IU/ml.

In some embodiments, the culture media further includes Dynabeads™ Human T-Activator.

In some embodiments, splitting the cultured cells, e.g., step (c), requires that half of the media is fresh and half of the media is original. In some embodiments, the media must be changed (e.g., 50% fresh) every 24 hours. In some embodiments, the media must be changed (e.g., 50% fresh) every 48 hours. In some embodiments, the media must be changed (e.g., 50% fresh) every 72 hours. In some embodiments, the media must be changed (e.g., 50% fresh) every 96 hours.

In some embodiments, the culturing and splitting steps, e.g., steps (b) and (c), are performed as described in Example 14.

In some embodiments, CD8⁺ cells are removed from the mixture in step (a). Removal of CD8⁺ T cells can be accomplished using any one of suitable cell removal methods known in the art (e.g., FACs). In some embodiments, removal of CD8⁺ T cells is accomplished as described in Example 13.

In some embodiments, an ex vivo expanded population of CD4⁺ T cells with cytolytic capabilities is obtained after about 1 months, about 2 months, about 3 months, about 4 months, after 5 months, or about 6 months. In some embodiments, an ex vivo expanded population of CD4⁺ T cells with cytolytic capabilities is obtained after 1 month. In some embodiments, an ex vivo expanded population of CD4⁺ T cells with cytolytic capabilities is obtained after 2 months. In some embodiments, an ex vivo expanded population of CD4⁺ T cells with cytolytic capabilities is obtained after 3 month.

As described above, to determine cytolytic capability (e.g., the ability to induce cell death in target cells), various suitable methods known in the art may be used. Non-limiting examples of methods suitable for determination of cytolytic capability of the cytolytic CD4 cells disclosed herein include lysis of autologous tumor cells (e.g., primary or transformed cells), allogeneic human tumor cells, or mouse mastocytoma target cells (P815) loaded with anti-human CD3 antibodies. Accordingly, in some embodiments, cytolytic capabilities of the cytolytic CD4⁺ cells disclosed herein are detected by lysis of autologous tumor cells (e.g., primary or transformed cells). In some embodiments, cytolytic capabilities of the cytolytic CD4⁺ cells disclosed herein are detected by allogeneic human tumor cells. In some embodiments, cytolytic capabilities of the cytolytic CD4⁺ cells disclosed herein are detected by lysis of mouse mastocytoma target cells (P815) loaded with anti-human CD3 antibodies.

In some embodiments, the enriched population of CD4⁺ T cells with cytolytic capabilities express one or more markers selected from the group consisting of GZMA, GZMB, GZMH, GZMK, KLRB1, KLRD1, GNLY, NKG7, CCL4, CCL5, LTB, CXCR4, CXCR6, PRF1, KLRG1, LAG3, CXCL13, and combinations of any thereof.

In some embodiments, cytolytic capabilities are detected by lysis of autologous tumor cells (e.g., primary or transformed cells), allogeneic human tumor cells, or mouse mastocytoma target cells (P815) loaded with anti-human CD3 antibodies. In some embodiments, cytolytic capabilities are detected by lysis of autologous tumor cells (e.g., primary or transformed). In some embodiments, cytolytic capabilities are detected by allogeneic human tumor cells. In some embodiments, cytolytic capabilities are detected by or mouse mastocytoma target cells (P815) loaded with anti-human CD3 antibodies. In some embodiments, cytolytic capabilities are detected as described in Example 15.

### IV. Methods of Use

The ex vivo populations of CD4⁺ T cells and the pharmaceutical compositions in accordance with the present disclosure, and particularly the populations of cytolytic CD4⁺ T cells and the pharmaceutical compositions containing the same, can be particularly suited for therapeutic applications. In this context, some embodiments of the disclosure relate to a method for treating bladder cancer in an individual including administering to the individual a therapeutically effective amount of a population of cytolytic CD4⁺ T cells as disclosed herein and/or a pharmaceutical composition as disclosed herein.

Thus, it is contemplated that the ex vivo populations of CD4⁺ T cells and the pharmaceutical compositions provided herein may be useful for the treatment of bladder cancer. In one aspect, provided is a method of treating an individual having or suspected of having bladder cancer, the method including administering to the individual an effective amount of a population of cytolytic CD4⁺ T cells. In another aspect, a method of targeting cancer cells in an individual having or suspected of having bladder cancer is provided, the method including administering to the individual an effective amount of a population of cytolytic CD4⁺ T cells. In a further aspect, a method of providing cell therapy to an individual having or suspected of having bladder cancer, the method including administering to the individual an effective amount of a population of cytolytic CD4⁺ T cells is provided.

Bladder cancer that can be treated with methods described herein include, but are not limited to, metastatic bladder cancer, non-muscle-invasive bladder cancer, or bladder cancer that is refractory to a standard therapy (such as BCG) or recurrent after the standard therapy. In some embodiments, the bladder cancer is BCG-refractory non-muscle-invasive bladder cancer. In some embodiments, the bladder cancer is platinum-refractory bladder cancer. In some embodiments, the bladder cancer is platinum-refractory metastatic urothelial carcinoma.

The methods described herein can be used for any one or more of the following purposes: alleviating one or more symptoms of bladder cancer, delaying progressing of bladder cancer, shrinking tumor size in bladder cancer patient, inhibiting bladder cancer tumor growth, prolonging overall survival, prolonging disease-free survival, prolonging time to bladder disease progression, preventing or delaying bladder cancer metastasis, reducing (such as eradiating) preexisting bladder cancer metastasis, reducing incidence or burden of preexisting bladder cancer metastasis, preventing recurrence of bladder cancer.

In some embodiments, the individual can be a human. In some embodiments, the human can be a human patient who has, who is suspected of having, or who may be at high risk for developing bladder cancer. In some embodiments, the individual can be a patient under the care of a physician.

The terms “administration” and “administering”, as used herein, refer to the delivery of a bioactive composition or formulation by an administration route including, but not limited to, oral, intravenous, intra-arterial, intramuscular, intraperitoneal, subcutaneous, intramuscular, and topical administration, or combinations thereof. The term includes, but is not limited to, administering by a medical professional and self-administering. One of ordinary skill in the art would be familiar with techniques for administering CD4⁺ cells and CD4⁺ cell-containing compositions to an individual. Furthermore, one of ordinary skill in the art would be familiar with techniques and pharmaceutical reagents necessary for preparation of these cells and cell-containing compositions prior to administration to an individual.

The term “effective amount,” “therapeutically effective amount” or “pharmaceutically effective amount” is an amount sufficient for a composition to accomplish a stated purpose relative to the absence of the composition (e.g., achieve the effect for which it is administered, treat a disease, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition or a “therapeutically effective amount” will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In reference to bladder cancer, an effective amount includes an amount sufficient to cause a tumor to shrink and/or to decrease the growth rate of the tumor (such as to suppress tumor growth) or to prevent or delay other unwanted cell proliferation in bladder cancer. In some embodiments, an effective amount is an amount sufficient to delay development of bladder cancer. In some embodiments, an effective amount is an amount sufficient to prevent or delay recurrence. In some embodiments, an effective amount is an amount sufficient to reduce recurrence rate in the individual. An effective amount can be administered in one or more administrations. In the case of bladder cancer, the effective amount of the drug or composition may: (i) reduce the number of bladder cancer cells; (ii) reduce tumor size; (iii) inhibit, retard, slow to some extent and preferably stop bladder cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; (vii) reducing recurrence rate of tumor, and/or (viii) relieve to some extent one or more of the symptoms associated with bladder cancer.

In some embodiments, the term “therapeutically effective amount” as used herein means the amount of a population of cytolytic CD4⁺ T cells when administered to a mammal, for example, a human, in need of such treatment, is sufficient to treat bladder cancer in the individual.

The methods described herein are useful for various aspects of bladder cancer treatment. In some embodiments, a method of inhibiting bladder cancer cell proliferation (such as bladder cancer tumor growth) in an individual includes administering to the individual an effective amount of a composition including an ex vivo population of cytolytic CD4⁺ T cells and/or a pharmaceutical composition in accordance with the present disclosure. In some embodiments, at least about 10% (including for example at least about any of 20%, 30%, 40%, 60%, 70%, 80%, 90%, or 100%) cell proliferation is inhibited.

In some embodiments, a method of inhibiting bladder cancer tumor metastasis in an individual includes administering to the individual an effective amount of a composition including an ex vivo population of cytolytic CD4⁺ T cells and/or a pharmaceutical composition in accordance with the present disclosure. In some embodiments, at least about 10%, such as for example at least about any of 20%, 30%, 40% metastasis is inhibited. In some embodiments, at least about 60%, 70%, 80%, 90%, or 100% metastasis is inhibited. In some embodiments, the composition including the ex vivo population of cytolytic CD4⁺ T cells and/or a pharmaceutical composition is administered by intravenous administration.

In some embodiments, a method of reducing (such as eradiating) preexisting bladder cancer tumor metastasis (such as pulmonary metastasis or metastasis to the lymph node) in an individual, including administering to the individual an effective amount of a composition including an ex vivo population of cytolytic CD4⁺ T cells and/or a pharmaceutical composition in accordance with the present disclosure. In some embodiments, at least about 10%, such as for example at least about any of 20%, 30%, 40%, 60%, 70%, 80%, 90%, or 100%, of metastasis may be reduced. In some embodiments, the composition including the ex vivo population of cytolytic CD4⁺ T cells and/or a pharmaceutical composition is administered by intravenous administration.

In some embodiments, the methods described herein result in prolonging survival of an individual having bladder cancer. In some embodiments, the method prolongs the survival of the individual by at least any of 1, 2, 3, 4, 5, 6, 7, 8, 9, 10, 11, 12, 18, or 24 month. In some embodiments, the methods described herein result in alleviating one or more symptoms in an individual having bladder cancer. In some embodiments, the methods described herein result in reducing incidence or burden of preexisting bladder cancer tumor metastasis (such as pulmonary metastasis or metastasis to the lymph node) in an individual. In some embodiments, the methods described herein result prolonging time to disease progression of bladder cancer in an individual. In some embodiments, the method prolongs the time to disease progression by at least any of 1, 2, 3, 4, 5, 6, 7, 8, 9, 10, 11, or 12 weeks. In some embodiments, the methods described herein result in reducing bladder cancer tumor size in an individual. This is because one indication of successful treatment may be a reduction in the size of the bladder cancer tumor. A reduction in tumor size may occur through the killing of bladder cancer cells. Thus, in some embodiments, one or more bladder cancer cells is destroyed.

In an embodiment, the population of cytolytic CD4⁺ T cells is an ex vivo population of cytolytic CD4⁺ T cells. In an embodiment, the CD4⁺ T cells express one or markers selected from the group consisting of GZMA, GZMB, GZMH, GZMK, KLRB1, KLRD1, GNLY, NKG7, CCL4, CCL5, LTB, CXCR4, CXCR6, PRF1, KLRG1, LAG3, CXCL13, and combinations of any thereof, and wherein the CD4⁺ T cells have cytolytic capabilities. In some embodiments, the population of cytolytic CD4⁺ T cells is an ex vivo population of cytolytic CD4⁺ T cells as described herein, including embodiments thereof.

In some embodiments of the treatment methods, the CD4⁺ T cells express the immune checkpoint marker LAG3 but lack expression of one or more additional immune checkpoint markers. In some embodiments, the additional immune checkpoint markers not expressed by the CD4⁺ T cells are selected from the group consisting of IL2RA/CD25, TNFRSF4/OX40, TNFRSF9/4-1BB, TNFRSF18/GITR, CD278/ICOS, TIGIT, and combinations of any thereof. In some embodiments, the CD4⁺ T cells further express heat shock proteins and/or IFN-gamma.

An effective amount of the CD4⁺ T cells disclosed herein can be determined based on the intended goal, for example tumor regression. For example, where existing cancer is being treated, the number of cells to be administered may be greater than where administration of the CD4⁺ T cells disclosed herein is for prevention of bladder cancer. One of ordinary skill in the art would be able to determine the number of cells to be administered and the frequency of administration in view of this disclosure. The quantity to be administered, both according to number of treatments and dose, also depends on the individual to be treated, the state of the individual, and the protection desired. Precise amounts of the therapeutic composition also depend on the judgment of the practitioner and are peculiar to each individual. Frequency of administration could range from 1-2 days, to 2-6 hours, to 6-10 hours, to 1-2 weeks or longer depending on the judgment of the practitioner.

Longer intervals between administration and lower numbers of CD4⁺ T cells disclosed herein may be employed where the goal is prevention. For instance, numbers of CD4⁺ T cells administered per dose may be 50% of the dose administered in treatment of active disease, and administration may be at weekly intervals. One of ordinary skill in the art, in light of this disclosure, would be able to determine an effective number of CD4⁺ T cells and frequency of administration. This determination would, in part, be dependent on the particular clinical circumstances that are present (e.g., type of cancer, severity of cancer).

In some embodiments, it may be desirable to provide a continuous supply of the therapeutic compositions to the individual. In some embodiments, continuous perfusion of the region of interest (such as the tumor) may be suitable. The time period for perfusion would be selected by the clinician for the particular individual and situation, but times could range from about 1-2 hours, to 2-6 hours, to about 6-10 hours, to about 10-24 hours, to about 1-2 days, to about 1-2 weeks or longer. Generally, the dose of the therapeutic composition via continuous perfusion will be equivalent to that given by single or multiple injections, adjusted for the period of time over which the doses are administered.

In some embodiments, administration is by bolus injection. In some embodiments, administration is by intravenous infusion. In some embodiments, an ex vivo population of CD4⁺ cells is administered in a dosage of about 1×10² cells, 1×10³ cells, 1×10⁴ cells, 1×10⁵ cells, 1×10⁶ cells, 1×10⁷ cells or more, or in a range of about 1×10³ to 1×10⁴ cells, 1×10³ to 1×10⁵ cells, 1×10³ to 1×10⁶ cells, 1×10⁴ to 1×10⁵ cells, or 1×10⁵ to 1×10⁶ cells, 1×10⁶ to 1×10⁷ cells, 1×10⁷ to 1×10⁸ cells. In some embodiments, the CD4⁺ cells are administered in a single administration. In some embodiments, CD4⁺ cells are administered in multiple administrations, (e.g., once or more per week for one or more weeks). In some embodiments, doses are administered about every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more days. In some embodiments, there are 1, 2, 3, 4, 5, 6, 7, 8, 9, 10, or more total doses. In some embodiments, 4 doses are administered, with a 3 week span between doses.

In some embodiments, the CD4⁺ cell-containing compositions can be an aqueous composition that includes the CD4⁺ cells of the disclosure. Aqueous compositions of the present disclosure contain an effective amount of a population of the CD4⁺ cells disclosed herein in a pharmaceutically acceptable carrier or aqueous medium. Thus, the “pharmaceutical preparation” or “pharmaceutical composition” of the disclosure can include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the CD4⁺ cells disclosed herein, its use in the manufacture of the pharmaceutical compositions is contemplated. Supplementary active ingredients can also be incorporated into the compositions. For human administration, preparations should meet sterility, pyrogenicity, general safety, and purity standards as required by the FDA Center for Biologics.

The CD4⁺ cells can generally be formulated for administration by any known route, such as parenteral administration. Determination of the number of cells to be administered can be made by one of skill in the art, and can in part be dependent on the extent and severity of cancer, and whether the CD4⁺ cells are being administered for treatment of existing cancer or prevention of cancer. The preparation of the pharmaceutical composition containing the CD4⁺ cells of the disclosure will be known to those of skill in the art in light of the present disclosure.

Upon formulation, pharmaceutical compositions can be administered in a manner compatible with the dosage formulation and in such amount as is therapeutically effective. The formulations are easily administered in a variety of dosage forms, such as the type of injectable solutions described above. For parenteral administration, the pharmaceutical compositions including the CD4⁺ cells disclosed herein should be suitably buffered. The CD4⁺ cells may be administered with other agents that are part of the therapeutic regiment of the individual, such as other immunotherapy or chemotherapy.

The CD4⁺ cells and/or pharmaceutical compositions (described herein can be used to stimulate the proliferation of one or more immune cells, for example, one or more of CD3⁺CD8⁺ T cells, CD3⁺CD4⁺ T cells, memory CD8⁺ T cells, NK cells, and NKT cells relative to the proliferation of these cells in individuals who have not been administered one of the compositions disclosed herein. The immune cells can be stimulated to proliferate up to about 20 fold, such as any of about 2 fold, 3 fold, 4 fold, 5 fold, 6 fold, 7 fold, 8 fold, 9 fold, 10 fold, 11 fold, 12 fold, 13 fold, 14 fold, 15 fold 16 fold, 17 fold, 18 fold, 19 fold, or 20 fold or higher compared to the proliferation of comparable cells in individuals who have not been administered one of the vaccine or cell-containing compositions described herein.

As discussed supra, any one of CD4⁺ cells and/or pharmaceutical compositions described herein can be administered in combination with one or more chemotherapeutics or anti-cancer agents or therapies. Administration “in combination with” one or more therapeutic agents includes simultaneous (concurrent) and consecutive administration in any order. In some embodiments, the one or more anti-cancer agents or therapies is selected from the group consisting of chemotherapy, radiotherapy, immunotherapy, hormonal therapy, toxin therapy, and surgery. “Chemotherapy” and “anti-cancer agent” are used interchangeably herein. Various classes of anti-cancer agents can be used. Non-limiting examples include: alkylating agents, antimetabolites, anthracyclines, plant alkaloids, topoisomerase inhibitors, podophyllotoxin, antibodies (e.g., monoclonal or polyclonal), tyrosine kinase inhibitors (e.g., imatinib mesylate such as Gleevec® or Glivec®), hormone treatments, soluble receptors and other antineoplastics.

Topoisomerase inhibitors are also another class of anti-cancer agents that can be used herein. Topoisomerases are essential enzymes that maintain the topology of DNA. Inhibition of type I or type II topoisomerases interferes with both transcription and replication of DNA by upsetting proper DNA supercoiling. Some type I topoisomerase inhibitors include camptothecins: irinotecan and topotecan. Examples of type II inhibitors include amsacrine, etoposide, etoposide phosphate, and teniposide. These are semisynthetic derivatives of epipodophyllotoxins, alkaloids naturally occurring in the root of American Mayapple (Podophyllum peltatum).

Antineoplastics include the immunosuppressant dactinomycin, doxorubicin, epirubicin, bleomycin, mechlorethamine, cyclophosphamide, chlorambucil, ifosfamide. The antineoplastic compounds generally work by chemically modifying a cell's DNA.

Alkylating agents can alkylate many nucleophilic functional groups under conditions present in cells. Cisplatin and carboplatin, and oxaliplatin are alkylating agents. They impair cell function by forming covalent bonds with the amino, carboxyl, sulfhydryl, and phosphate groups in biologically important molecules.

Vinca alkaloids bind to specific sites on tubulin, inhibiting the assembly of tubulin into microtubules (M phase of the cell cycle). The vinca alkaloids include: vincristine, vinblastine, vinorelbine, and vindesine.

Anti-metabolites resemble purines (azathioprine, mercaptopurine) or pyrimidine and prevent these substances from becoming incorporated in to DNA during the “S” phase of the cell cycle, stopping normal development and division. Anti-metabolites also affect RNA synthesis.

Plant alkaloids and terpenoids are obtained from plants and block cell division by preventing microtubule function. Since microtubules are vital for cell division, without them, cell division cannot occur. The main examples are vinca alkaloids and taxanes.

Podophyllotoxin is a plant-derived compound which has been reported to help with digestion as well as used to produce two other cytostatic drugs, etoposide and teniposide. They prevent the cell from entering the G1 phase (the start of DNA replication) and the replication of DNA (the S phase).

Taxanes as a group includes paclitaxel and docetaxel. Paclitaxel is a natural product, originally known as Taxol and first derived from the bark of the Pacific Yew tree. Docetaxel is a semi-synthetic analogue of paclitaxel. Taxanes enhance stability of microtubules, preventing the separation of chromosomes during anaphase.

In some embodiments, the anti-cancer agents can be selected from remicade, docetaxel, celecoxib, melphalan, dexamethasone (Decadron®), steroids, gemcitabine, cisplatinum, temozolomide, etoposide, cyclophosphamide, temodar, carboplatin, procarbazine, gliadel, tamoxifen, topotecan, methotrexate, gefitinib (Iressa®), taxol, taxotere, fluorouracil, leucovorin, irinotecan, xeloda, CPT-11, interferon alpha, pegylated interferon alpha (e.g., PEG INTRON-A), capecitabine, cisplatin, thiotepa, fludarabine, carboplatin, liposomal daunorubicin, cytarabine, doxetaxol, pacilitaxel, vinblastine, IL-2, GM-CSF, dacarbazine, vinorelbine, zoledronic acid, palmitronate, biaxin, busulphan, prednisone, bortezomib (Velcade®), bisphosphonate, arsenic trioxide, vincristine, doxorubicin (Doxil®), paclitaxel, ganciclovir, adriamycin, estrainustine sodium phosphate (Emcyt®), sulindac, etoposide, and combinations of any thereof.

In other embodiments, the anti-cancer agent can be selected from bortezomib, cyclophosphamide, dexamethasone, doxorubicin, interferon-alpha, lenalidomide, melphalan, pegylated interferon-alpha, prednisone, thalidomide, or vincristine.

In some embodiments, the methods of treatment as described herein further include administration of a compound that inhibits one or more immune checkpoint molecules. In some embodiments, the one or more immune checkpoint molecules include one or more of CTLA4, PD-1, PD-L1, A2AR, B7-H3, B7-H4, TIM3, and combinations of any thereof. In some embodiments, the compound that inhibits the one or more immune checkpoint molecules includes an antagonistic antibody. In some embodiments, the antagonistic antibody is ipilimumab, nivolumab, pembrolizumab, durvalumab, atezolizumab, tremelimumab, or avelumab.

In some aspects, the one or more anti-cancer therapy is radiation therapy. As used herein, the term “radiation therapy” refers to the administration of radiation to kill cancerous cells. Radiation interacts with molecules in the cell such as DNA to induce cell death. Radiation can also damage the cellular and nuclear membranes and other organelles. Depending on the radiation type, the mechanism of DNA damage may vary as does the relative biologic effectiveness. For example, heavy particles (e.g. protons, neutrons) damage DNA directly and have a greater relative biologic effectiveness. Electromagnetic radiation results in indirect ionization acting through short-lived, hydroxyl free radicals produced primarily by the ionization of cellular water. Clinical applications of radiation consist of external beam radiation (from an outside source) and brachytherapy (using a source of radiation implanted or inserted into the individual). External beam radiation consists of X-rays and/or gamma rays, while brachytherapy employs radioactive nuclei that decay and emit alpha particles, or beta particles along with a gamma ray. Radiation also contemplated herein includes, for example, the directed delivery of radioisotopes to cancer cells. Other forms of DNA damaging factors are also contemplated herein such as microwaves and UV irradiation.

Radiation may be given in a single dose or in a series of small doses in a dose-fractionated schedule. The amount of radiation contemplated herein ranges from about 1 to about 100 Gy, including, for example, about 5 to about 80, about 10 to about 50 Gy, or about 10 Gy. The total dose may be applied in a fractioned regime. For example, the regime may include fractionated individual doses of 2 Gy. Dosage ranges for radioisotopes vary widely, and depends on the half-life of the isotope and the strength and type of radiation emitted. When the radiation includes use of radioactive isotopes, the isotope may be conjugated to a targeting agent, such as a therapeutic antibody, which carries the radionucleotide to the target tissue (e.g., tumor tissue).

Surgery described herein includes resection in which all or part of a cancerous tissue is physically removed, exercised, and/or destroyed. Tumor resection refers to physical removal of at least part of a tumor. In addition to tumor resection, treatment by surgery includes laser surgery, cryosurgery, electrosurgery, and micropically controlled surgery (Mohs surgery). Removal of precancers or normal tissues is also contemplated herein.

In some embodiments, the methods of the disclosure further include administering to the individual a second anti-cancer agent or therapy. In some embodiments, the second anti-cancer agent or therapy is selected from the group consisting of chemotherapy, radiotherapy, immunotherapy, hormonal therapy, toxin therapy, and surgery. In some embodiments, the first agent and the second anti-cancer agent or therapy are administered concomitantly. In some embodiments, the first agent and the second anti-cancer agent or therapy are administered sequentially. In some embodiments, the first agent is administered before the second anti-cancer agent or therapy. In some embodiments, the first agent or therapy is administered before and/or after the second anti-cancer agent or therapy. In some embodiments, the first agent and the second anti-cancer agent or therapy are administered in rotation. In some embodiments, the first agent is administered at the same time as the second anti-cancer agent or therapy. In some embodiments, the first agent and the second anti-cancer agent or therapy are administered together in a single formulation.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

Additional embodiments are disclosed in further detail in the following examples, which are provided by way of illustration and are not in any way intended to limit the scope of this disclosure or the claims.

### EXAMPLES

In the Examples described below, droplet single-cell RNA and paired T cell receptor (TCR) sequencing were performed on CD4⁺ and CD8⁺ T cells isolated from the blood, tumors, and paired adjacent non-malignant tissue from patients with localized muscle-invasive bladder cancer. Patients who received anti-PD-L1 treatment before surgery were also assessed. As discussed below, in addition to known CD4⁺ regulatory and central memory populations, it was observed that bladder tumors are also infiltrated by novel populations of CD4⁺ T cells expressing cytotoxic effector and granule-associated proteins. These cytotoxic CD4⁺ T cells were found to be phenotypically heterogeneous, clonally expanded in the tumor environment, and could kill autologous tumor cells. Moreover, these cytotoxic CD4⁺ T cells circulate, including specific subsets expressing GZMB, NKG7, and GZMK that share identical TCR clonotypes in tumors and the periphery. In response to anti-PD-L1 therapy, clonotype sharing between cytotoxic CD4⁺ subtypes in the tumor is increased consistent with clonal expansion. Moreover, anti-PD-L1 treatment may increase the abundance of specific circulating cytotoxic CD4⁺ T cells expressing GZMB, NKG7, and GZMK. Although the intratumoral CD8⁺ TCR repertoire is more oligoclonal overall, the frequency and repertoire of individual CD8⁺ populations are not altered in tumor. However, specific CD8⁺ populations in the periphery are clonally expanded, including a GZMK-expressing population that shares specificity with tumor-infiltrating CD8⁺. These findings reveal the importance of cytotoxic CD4⁺ effector T cells in both the tumor and circulation of bladder cancer patients that can mediate tumor killing and are modulated by PD-1 blockade during response to therapy.

### Example 1

### Methods for Prospective Isolation of Tumor-Reactive Cytotoxic CD4⁺ T Cells for Anti-Cancer Therapy

Some embodiments of the disclosure relate to methods involving the use of specific markers identified from a novel tumor-infiltrating subset of cytotoxic CD4⁺ T lymphocytes in bladder cancer, to prospectively identify and isolate these effector immune cells from patient samples (blood and/or tumor) for ex vivo expansion, optimization, and readministration to patients as adoptive cell therapy for bladder cancer. These markers include a combination of GZMA, GZMB, GZMH, GZMK, KLRB1, KLRD1, GNLY, NKG7, CCL4, CCL5, LTB, CXCR4, CXCR6, PRF1, KLRG1, LAG3, and CXCL13.

Through the use of a novel combination of markers, the methods described herein allow for therapeutic isolation of a novel effector T cell population (e.g., cytotoxic CD4⁺ T cells) that possess more potent, or more specific, anti-tumor immune responses than existing cancer therapeutic approaches including adoptive T cell approaches focused on conventional cytotoxic CD8⁺ T cells.

The isolated novel tumor-infiltrating subset of cytotoxic CD4⁺ T lymphocytes as disclosed herein is contemplated to be useful for autologous T cell therapy in bladder cancer. Isolated tumor-reactive T cells may be engineered ex vivo to enhance anti-tumor activity and subsequently reintroduced into patients.

The cytotoxic potential of the CD4⁺ T cells identified by the markers can be confirmed in model organisms and in vitro experiments with CD4⁺ T cells and tumor targets from representative patients with solid bladder cancer tumors. The antigenic specificity of these isolated CD4⁺ T cells can also be identified.

Further, development of appropriate ex vivo culture/expansion conditions to generate sufficient numbers of cytotoxic CD4⁺ T cells for readministration to patients can be completed, in addition to preclinical and early phase clinical testing of safety and efficacy.

### Example 2

### Single-Cell Discrimination of Altered Human T Cell States Specific to the Bladder Tumor Microenvironment

Bladder cancer can be responsive to immunotherapies such as anti-PD-1 and anti-PD-L1 checkpoint inhibitors, but overall response rates are low. Tumor-resident T cells demonstrate considerable heterogeneity as far as antigenic repertoire and phenotype. Whether the bladder tumor environment is enriched for specific types of T cells with a particular functional profile and antigenic specificity, and whether these tumor-specific populations are associated with treatment or response to immunotherapy, remains unclear. We performed single-cell RNA sequencing of CD4⁺ and CD8⁺ T cells from localized human bladder tumors and paired adjacent non-malignant bladder. We also assessed resected bladder tumors treated with neoadjuvant chemotherapy or atezolizumab (anti-PD-L1) as part of an ongoing phase II trial. We find both known and unexpected CD4⁺ T cell functional populations that are specific to the tumor microenvironment, including regulatory T cells and a novel population of cytotoxic CD4⁺ T cells. Furthermore, by combining whole-transcriptome data with paired T cell receptor (TCR) identification on single cells, we find that tumor-specific CD4⁺ populations are clonally expanded and utilize an antigenic repertoire which is distinct from uninvolved bladder. These findings provide evidence for specialization of both phenotype and antigenic specificity of CD4⁺ T cells in the bladder tumor environment, and indicate that a limited number of tumor-specific antigens may drive in situ expansion and differentiation of specialized CD4⁺ subsets whose function may be critical to tumor control.

The intratumoral CD4⁺ compartment in bladder tumors demonstrates functional specialization (for Tregs and novel cytotoxic CD4⁺) as well as restricted antigenic specificity in these subsets. This suggests that a limited number of tumor-specific antigens may drive clonal expansion of these specialized tumor-resident CD4⁺ populations.

### Example 3

### CD4⁺ T Cells in Bladder Tumors are Composed of Multiple Populations of Regulatory and Cytotoxic CD4⁺ Cells

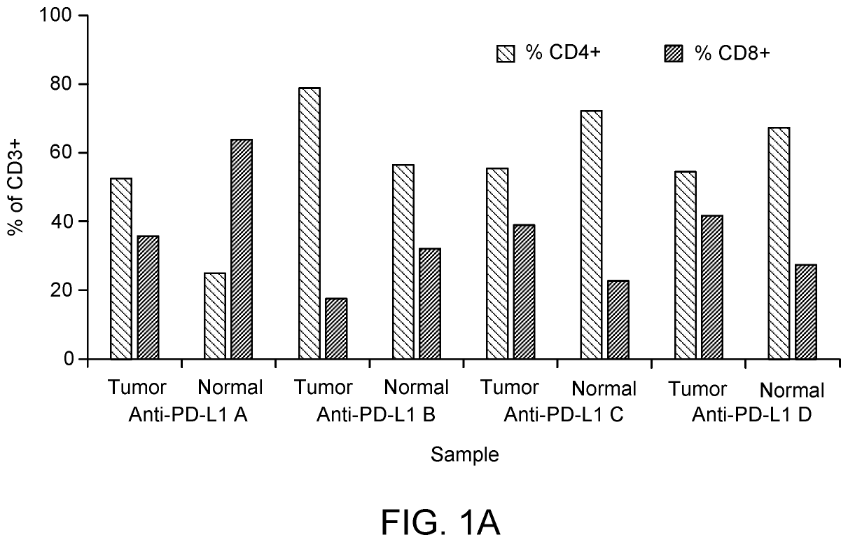

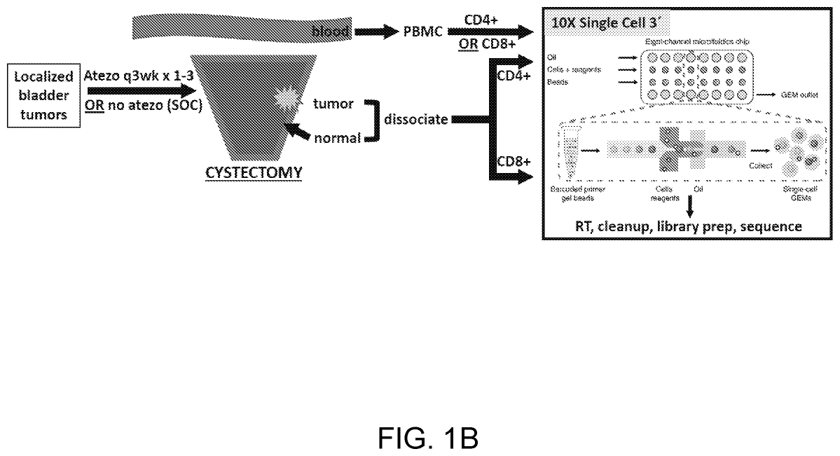

This Example describes the isolation and characterization of CD4⁺ T cells from patient populations. CD4⁺ T cells from tumor and adjacent normal tissue from localized bladder tumors (treated with atezolizumab/anti-PD-L1 or untreated) were isolated by FACS and subjected to scRNAseq (10× Genomics Single Cell 3′ Version 1) as shown in FIGS. 1A and 1B.

To assess the T cell composition of the tumor environment, the assessment of T cells from dissociated bladder tumors and adjacent uninvolved bladder tissues was performed using single cell techniques. As indicated in flow cytometry experiments, it was observed that the frequency of CD4⁺ T cells exceeds that of CD8⁺ T cells (FIG. 1A). In these experiments, the 10× Genomics Chromium platform was used to sequence 18,979 tumor- and 3,263 non-malignant-infiltrating CD4⁺ T cells isolated from 7 patients (see, FIG. 1A and Table 1). All samples were muscle-invasive bladder cancer (MIBC) derived from seven (7) patients: two standard-of-care untreated patients (“untreated”), one chemotherapy-treated patient (gemcitabine+carboplatin, “chemo”), and 4 anti-PD-L1-treated patients (“anti-PD-L1”) with detailed clinical annotations (see, e.g., Table 1).

In these experiments, pooled analysis of samples was conducted using Seurat v2.0 and canonical correlation analysis (CCA) to align multiple samples followed by unbiased clustering to identify functional populations.

Barcoded cDNA was amplified using consensus primers to generate libraries of TCR sequence encoded with 10× cell barcodes and the 10× unique molecular identifiers (UMI). After sequencing, miXCR was run for each individual cell (mixcr align and mixcr assemble) to assign clonotypes. Filtering was conducted to restrict analysis to clones supported by at least 2 UMIs; chimeric species (e.g., identical CDR3 sequences associated with multiple combinations of 10× barcodes+UMIs) were removed.

### Example 4

### Study Population

Table 1 provides information about the seven (7) patients used in this study, including patient ID, age, sex, presence and size of tumor at surgery, Path T stage, Path N stage, and the number of CD4 and CD8 cells isolated from the tumor, normal tissue, and blood.

TABLE 1

| | | | MIBC | | | | | | |
|---|---|---|---|---|---|---|---|---|---|
| | | | or | Neoadj | Tumor at | | Path T | Path N | # tumor |
| Pt ID | Age | M/F | NMIBC | tx | surgery? | | stage | stage | CD4 |
| Anti-PD-L1 A | 74 | M | MIBC | Atezo x | Y | (2.0 cm) | ypTa | ypN0 | 2396 |
| | | | | 1 | | | | | |
| Anti-PD-L1 B | 64 | M | MIBC | Atezo x | Y | (6.5 cm) | ypT4b | ypN2 | 2843 |
| | | | | 2 | | | | | |
| Anti-PD-L1 C | 68 | F | MIBC | Atezo x | Y | (6.8 cm) | ypT1 | ypN0 | 3694 |
| | | | | 2 | | | | | |
| Anti-PD-L1 D | 71 | M | MIBC | Atezo x | Y | (1.5 cm) | ypT2b | ypN0 | 3721 |
| | | | | 2 | | | | | |
| Chemo | 67 | F | MIBC | Chemo | Y | (<0.1 cm) | ypT1s | ypN0 | 1826 |
| Untreated A | 82 | M | MIBC | None | Y | (3 cm) | pT3b | pN0 | 3378 |
| Untreated B | 76 | M | MIBC | None | Y | (4 cm) | pT3b | pN0 | 1039 |

| | | | | | | | | |
|---|---|---|---|---|---|---|---|---|
| Healthy | NA | NA | NA | NA | NA | NA | NA | NA |

| | | # pre-tx | # post-tx | | | # pre-tx | # post-tx |
|---|---|---|---|---|---|---|---|
| | # normal | blood | blood | # tumor | # normal | blood | blood |
| Pt ID | CD4 | CD4 | CD4 | CD8 | CD8 | CD8 | CD8 |
| Anti-PD-L1 A | NA | 5509 | 5383 | 1301 | 93 | 2937 | 4089 |
| Anti-PD-L1 B | 441 | 834 | 7557 | 663 | NA | 732 | 5696 |
| Anti-PD-L1 C | 672 | 7031 | 7180 | 1402 | NA | 5479 | 4958 |
| Anti-PD-L1 D | 347 | 5343 | 4187 | 1023 | NA | 3827 | 2375 |
| Chemo | 1075 | NA | 6217 | 1975 | 437 | NA | 2813 |
| Untreated A | 135 | NA | 5818 | 1911 | NA | NA | 4399 |
| Untreated B | 365 | NA | 6323 | 1425 | 1564 | NA | 6262 |
| Healthy | NA | NA | 13634 | NA | NA | NA | 7054 |

### Example 5

### Intratumoral CD4⁺ Include Tregs and Novel Cytotoxic CD4⁺

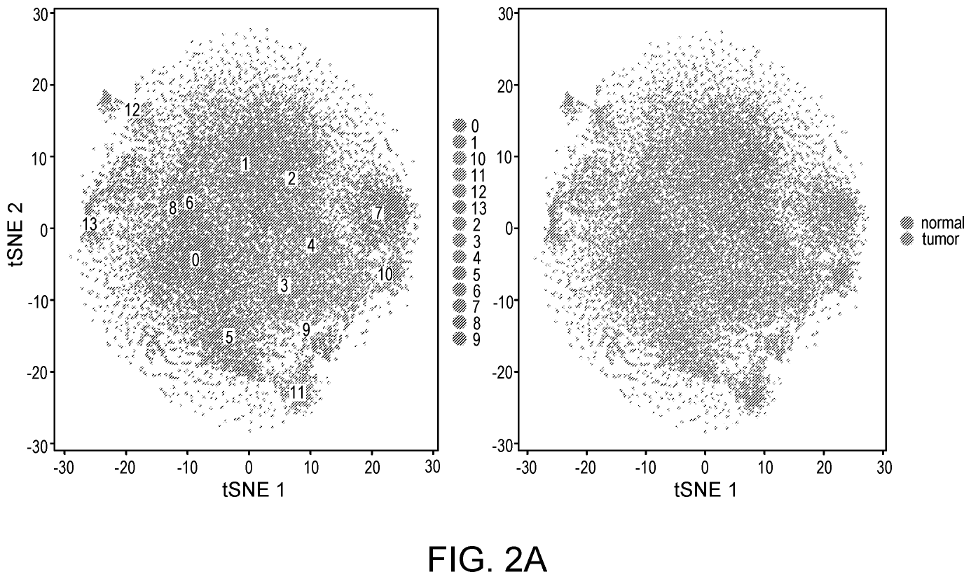

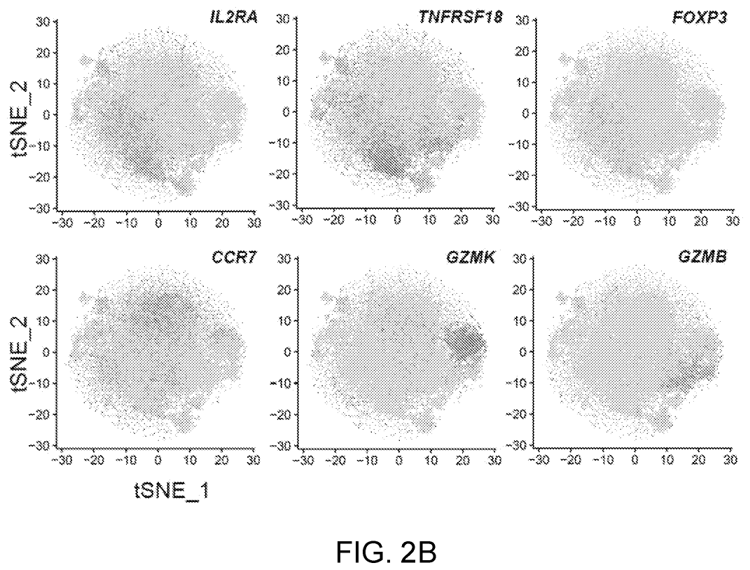

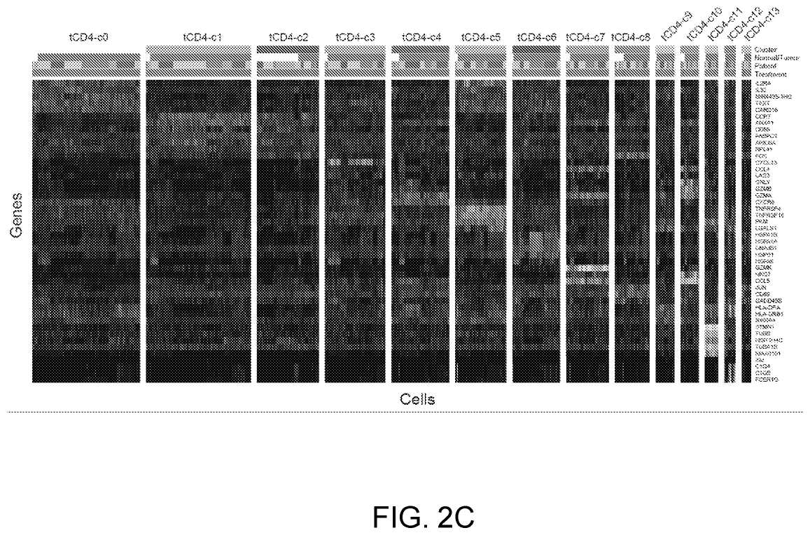

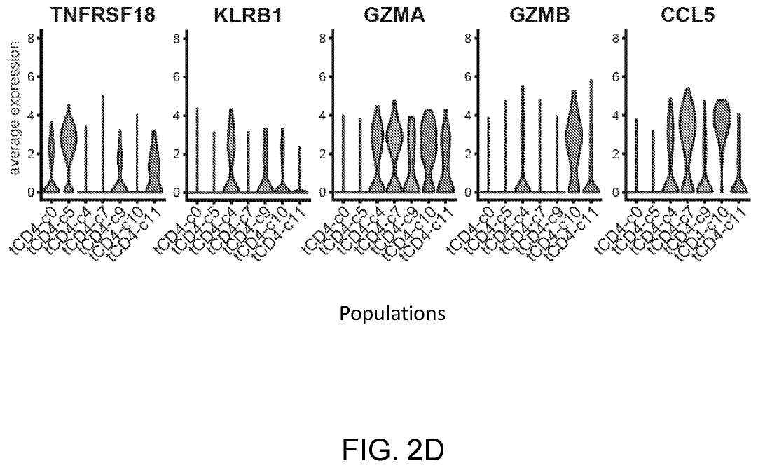

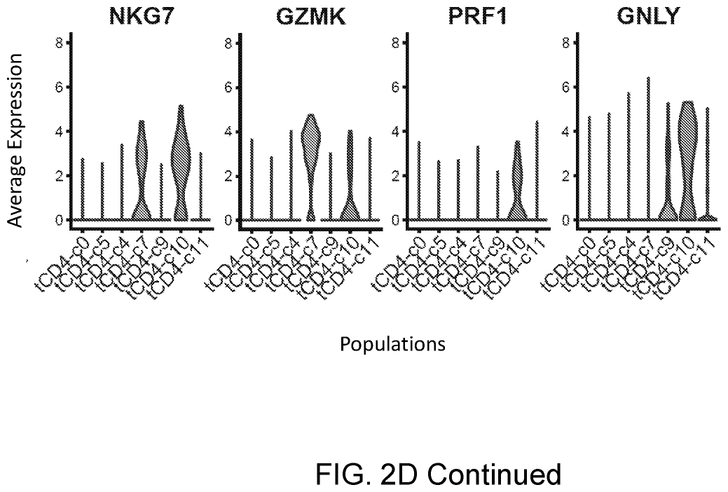

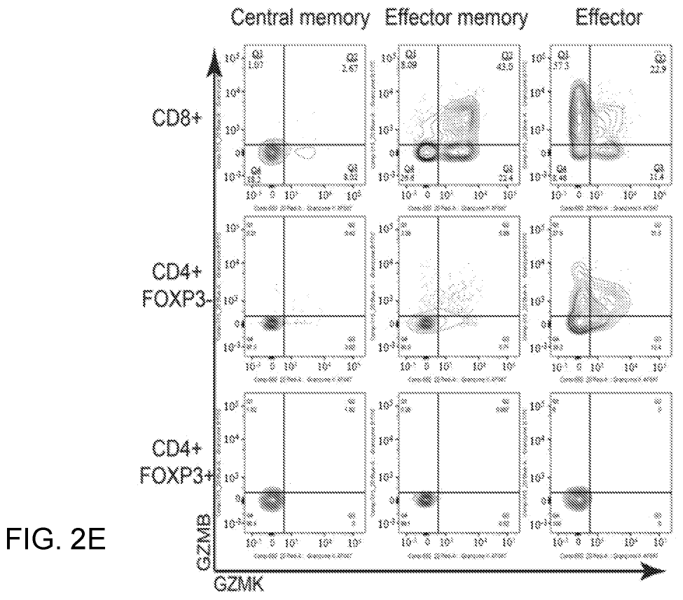

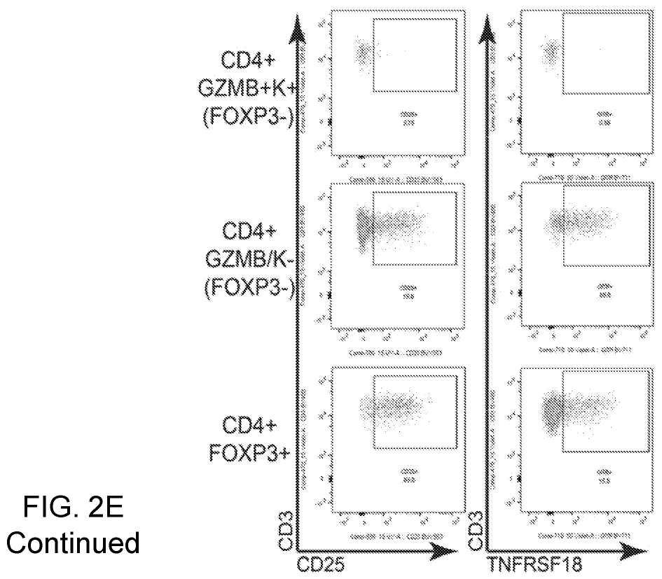

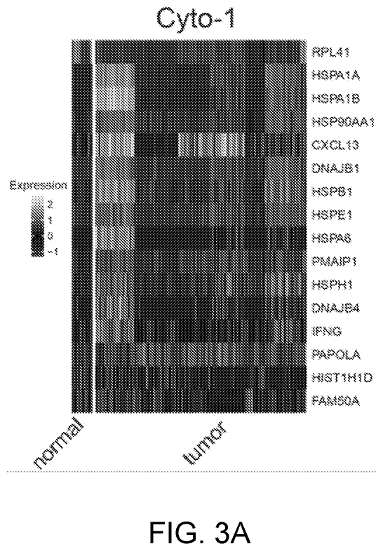

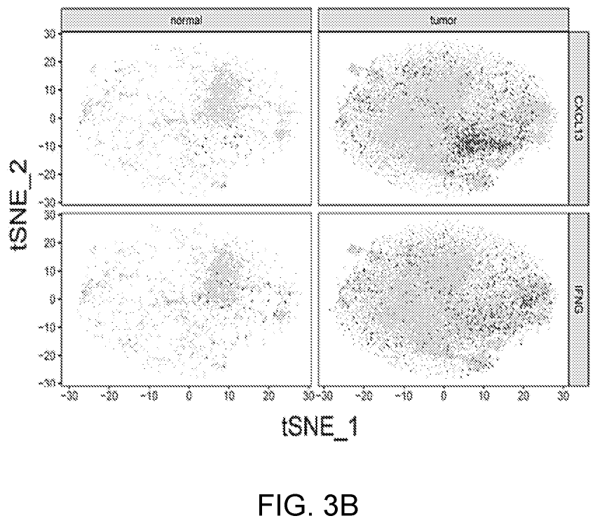

To assess the heterogeneity of CD4⁺ T cells across samples while controlling for technical and biological artifacts, the analysis was restricted to highly variable genes and used canonical correlation analysis (CCA) to identify common sources of variation among samples and projected the data onto maximally correlated subspaces. Following CCA, the k-nearest neighbor graph on a 20-dimensional manifold of the data was calculated and Louvain community detection was used to define clusters which were visualized using t-Stochastic Neighbor Embedding (tSNE). Tumor- and non-malignant-derived CD4⁺ form 14 clusters that are populated by cells from each individual sample without noticeable patient-specific artifacts. FIGS. 2A-2E show pooled analysis of CD4⁺ tumor-infiltrating lymphocytes (TILs) from 7 localized bladder tumors.

Each of 13 clusters were compared to a CCR7⁺ central memory population as reference (tCD4-c1) to focus on relative differences between clusters, and identified 1,117 genes that are differentially expressed in at least one cluster (P_{adj}<0.05, |log₂(FC)|>0.5 (see, e.g., Table 2 and FIGS. 2B-2C). Several canonical populations of CD4⁺ T cells could be identified. These include 2 populations of regulatory T cells (tCD4-c0, tCD4-c5) together constituting 27±1.8% (mean±s.e.m.) of tumor-infiltrating CD4⁺ cells, which express FOXP3 (tCD4-c0: log₂(FC)=0.62; tCD4-c5: log₂(FC)=0.73) and known immune checkpoints (tCD4-c0 and tCD4-c5: log₂(FC)>0.93 for IL2RA, TIGIT, TNFRSF4/9/18, CD27; FIG. 2C). tCD4-c5 was distinguished from tCD4-c0 based on higher expression of TNFRSF4/18 and LAG3 (log₂(FC) vs CCR7⁺ reference, P_{adj}<0.05 (see, e.g., Table 2). Also found was a population expressing high levels of CXCL13, LAG3 and IFNG (tCD4-c3: log₂(FC)=2.8, 1.5, 0.92), whose presence has been associated with improved outcomes in breast and gastric cancer, and also is found in microsatellite-unstable colorectal carcinoma which is an immune-responsive tumor. Other populations included an additional CCR7⁺ central memory population (tCD4-c2); 2 effector populations expressing either heat shock proteins with checkpoints such as TIGIT/IL2RA/TNFRSF18/TNFRSF4 (tCD4-c6: log₂(FC)>0.87) or CD69 (tCD4-c8: log₂(FC)=1.11) but not FOXP3 or granzymes (log₂(FC)<0.5); actively cycling cells (tCD4-c11) expressing proliferation markers (MKI67), microtubule-associated markers (e.g. STMN1/TUBB1), and DNA-binding proteins associated with cell cycle progression such as PCNA, HMGB1, and HMGB2; and a population of contaminating macrophages expressing CD14, CD68, and CST3 (tCD4-c12) (see, e.g., FIG. 2C).

Also found were 4 distinct populations of cytotoxic CD4⁺ in all samples constituting 23±2.3% of tumor-infiltrating CD4⁺ T cells. These populations all significantly expressed (log₂(FC)>0.5, P_{adj}<0.05) a core set of cytolytic effector molecules: GZMA and GZMB and the granule-associated GNLY which is a pore-forming protein known to function in pathogen killing (FIGS. 2B-2C). Individual cytotoxic populations overexpressed distinct cytolytic molecules: GZMB (tCD4-c4: log₂(FC)=1.7), GZMK (tCD4-c7: log 2(FC)=2.4), GNLY (tCD4-c9: log 2(FC)=1.5), and NKG7 (a granule protein that translocates to the surface of NK cells following target cell recognition suggesting a cytolytic role) (tCD4-c10: log₂(FC)=3.0). Specific cytotoxic populations also co-expressed additional genes which may contribute to anti-tumor effector function: GZMH and PRF1 (perforin) in tCD4-c7 and tCD4-c10, IFNG in tCD4-c3/tCD4-c7/tCD4-c10 populations, and CXCR6 in tCD4-c4 which is expressed in both regulatory and non-regulatory CD4 TILs from colorectal carcinoma, nasopharyngeal carcinoma, and renal cell carcinoma, and its ligand CXCL16 can mediate TIL chemotaxis. (FIGS. 2B-2C). Cytotoxic CD4⁺ populations expressed low levels of activation markers such as IL2RA in tCD4-c9 (log₂FC=0.57), and low levels of other checkpoints such as HAVCR2 (TIM3) in tCD4-c7 and tCD4-c10 (log 2FC=0.63-0.67), and TNFRSF18 (GITR) in tCD4-c4 (log 2FC=0.52). Similar populations are found with unbiased clustering without CCA alignment for paired tumor- and non-malignant-derived CD4⁺ cells from individual patients (see, e.g., FIGS. 2A-2E).

### Example 6

### Intratumoral CD8⁺ T Cells in Bladder Tumors Include Known Populations of MAIT, Effector, Central Memory, and Cycling T Cells

This Example describes experiments performed to demonstrate that intratumoral CD8⁺ T cells in bladder tumors include previously known categories of CD8⁺ T cells that are not enriched in tumor.

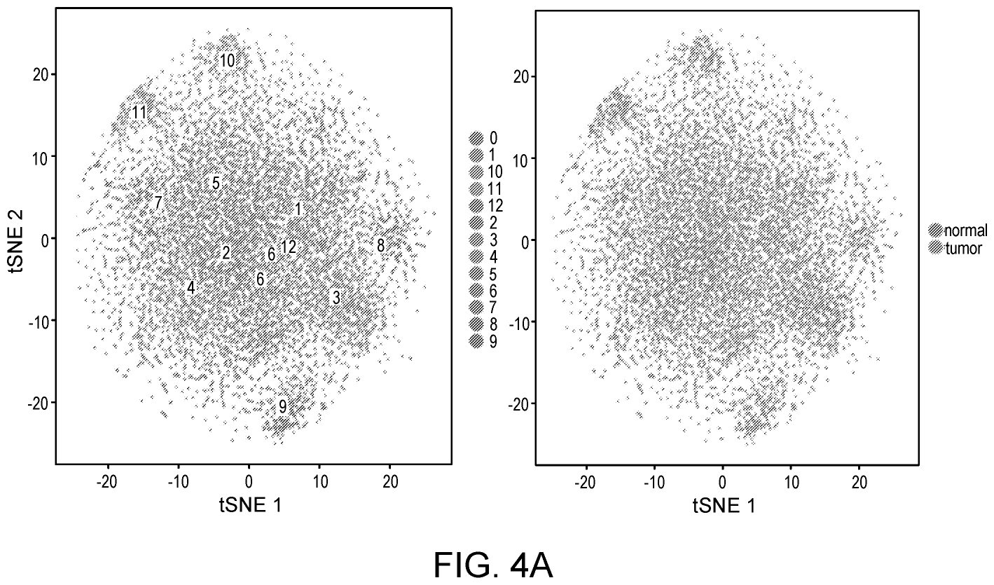

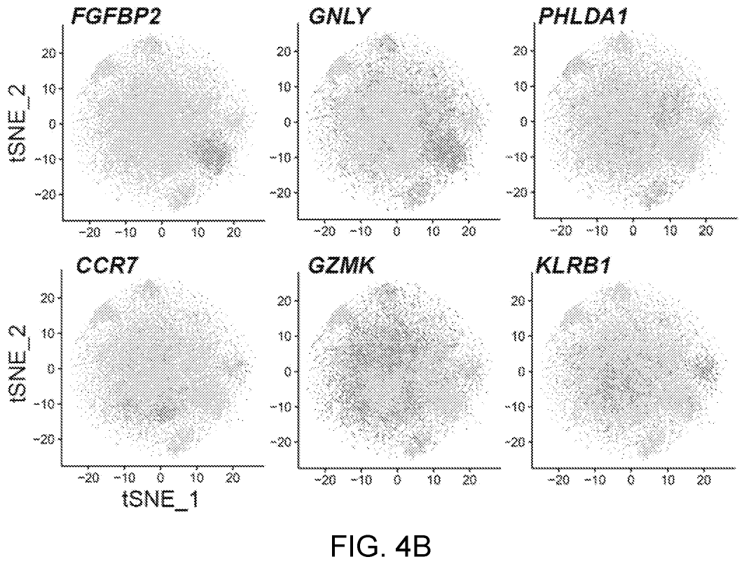

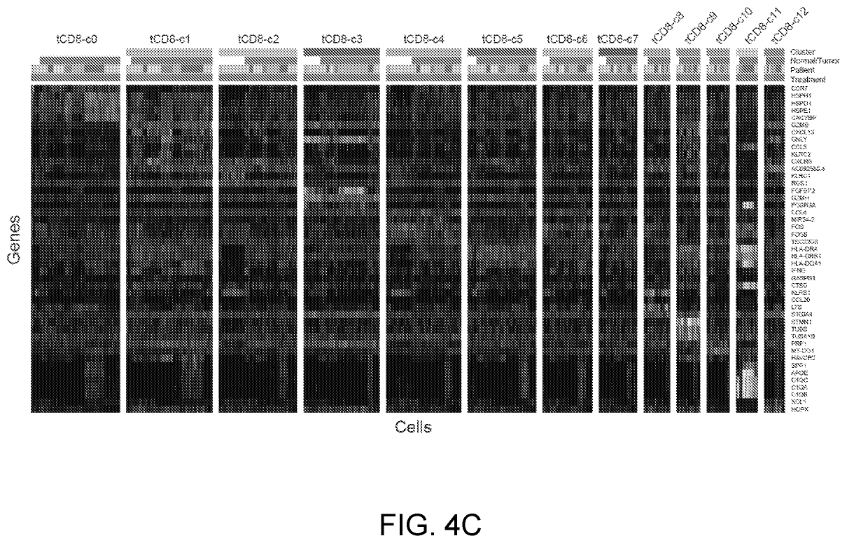

A total number of 10,145 tumor- and 2,288 non-malignant-derived CD8⁺ T cells from the same patients were sequenced and analyzed (see, Table 1) to identify 13 clusters with representation across individual patients (FIG. 4A). Using a CCR7⁺ central memory population as reference (tCD8-c0) and comparing each of the remaining 12 clusters, a total of 510 genes were identified as being differentially expressed in at least one cluster within tumor while being conserved across tumor and non-malignant compartments (P_{adj}<0.05, |log₂(FC)|>0.5). The identified populations included cells expressing ENTPD1 (tCD8-c1) previously described as tumor-reactive CD8⁺ T cells; effector cells expressing FGFBP2 (tCD8-c3 log₂(FC)=2.2) or activation markers such as MHC II (tCD8-c5: log₂(FC): 0.7-1.1) or CD69 (tCD8-c6: log₂(FC)=0.72); mucosal-associated invariant cells expressing KLRB1 (tCD8-c8: log₂(FC)=2.2) that frequently use the semi-invariant TCR alpha chains TRAV1-2/TRAJ33 in our own TCR data (see below) in agreement with published findings; cycling cells expressing STMN1/TUBB and MK167 similar to cycling CD4⁺ (tCD8-c9); and contaminating macrophages expressing CD14/CD68/CST3/CSF1R (tCD8-c 11) (see, e.g., FIGS. 4B-4C). Similar populations were also identified in the tumor environment of hepatocellular carcinoma based on scRNA-seq.

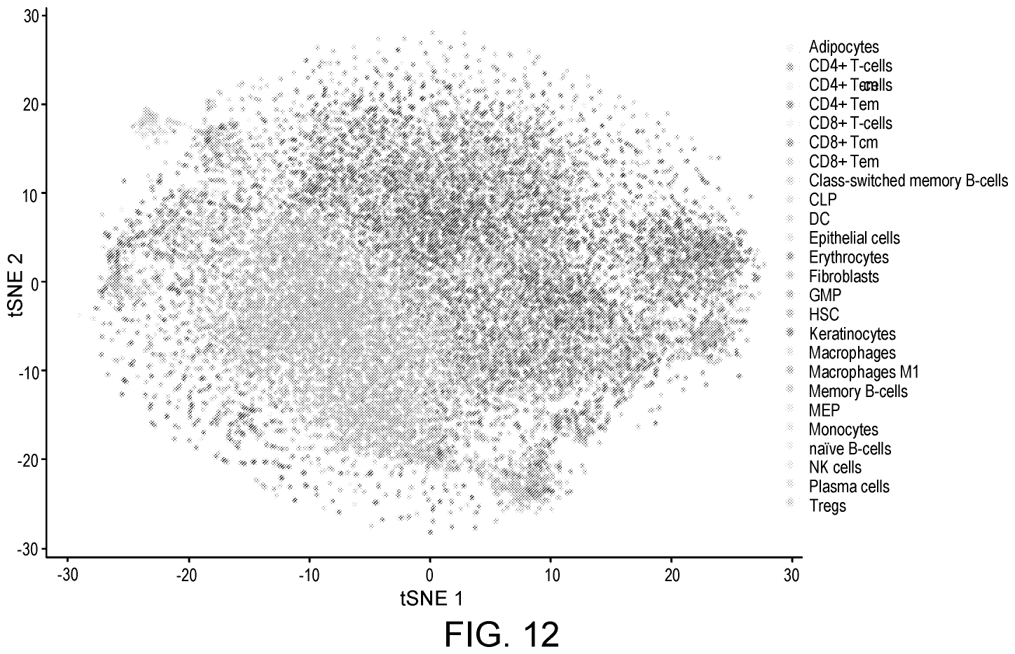

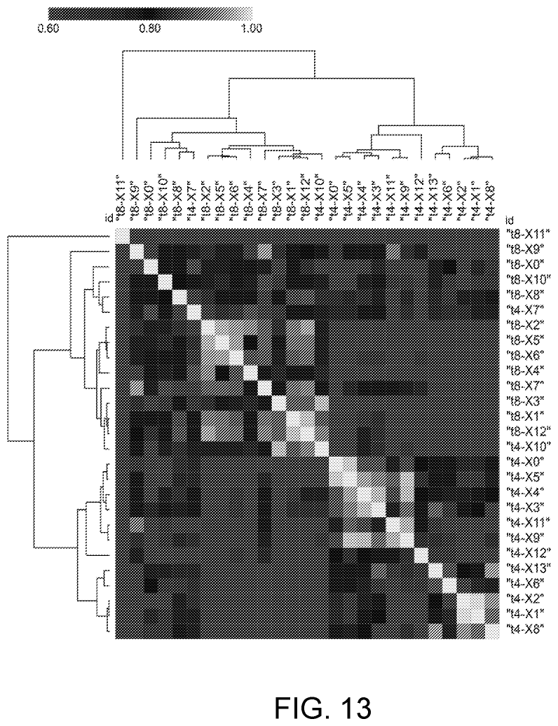

Annotation of clusters was supported by an independent analysis that assigns each single cell to the best-known immune subset based on gene expression (SingleR). This corroborates the identification of regulatory T cells and demonstrates that multiple cytotoxic CD4⁺ populations are most similar to CD8⁺ central or effector memory T cells, reinforcing their cytotoxicity profile (FIG. 12). In addition, a comparison of all CD4⁺ and CD8⁺ TIL clusters indicated that while the correlation is generally higher amongst clusters in the CD4⁺ and CD8⁺ compartments, cytotoxic CD4⁺ are an exception. The tCD4-c7 cytotoxic cells were found to be most correlated with tCD8-c4 (R=0.86) and the tCD4-c10 cytotoxic cells are most correlated with tCD8-c3 (R=0.94) (FIG. 13). Hence, unbiased dscRNAseq revealed that heterogeneous cytotoxic CD4⁺, a subset of which are closely related to conventional cytotoxic CD8⁺ based on their functional program, are an unexpected but frequent constituent of the bladder tumor microenvironment.

### Example 7

### Intratumoral CD4⁺ Compartment Exhibits a Predominance of Regulatory T Cells as Well as Enrichment of Activation Markers

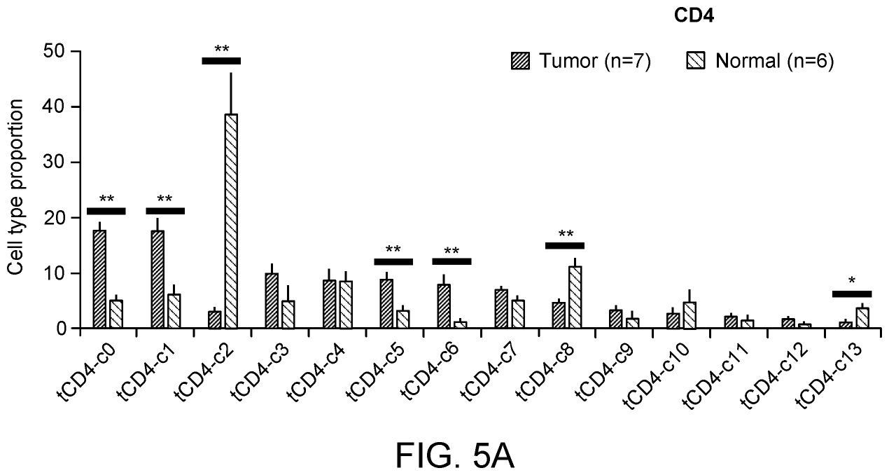

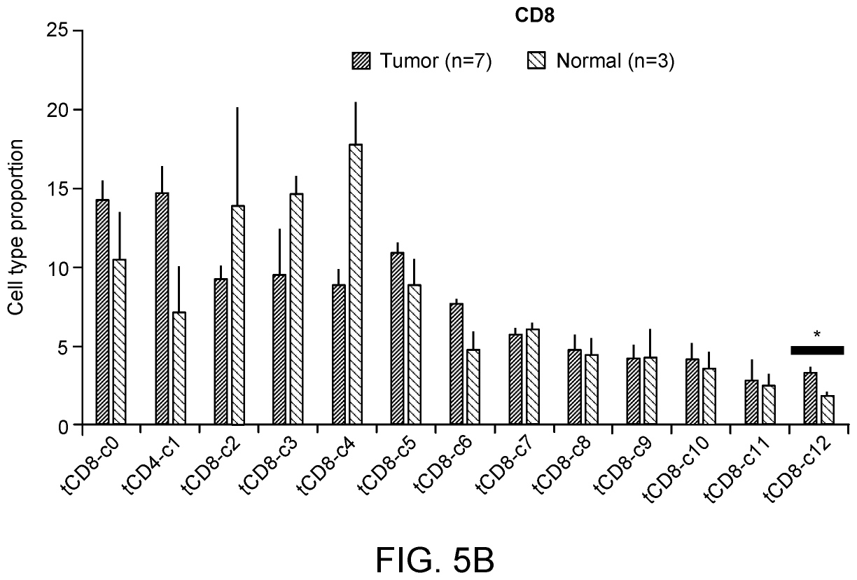

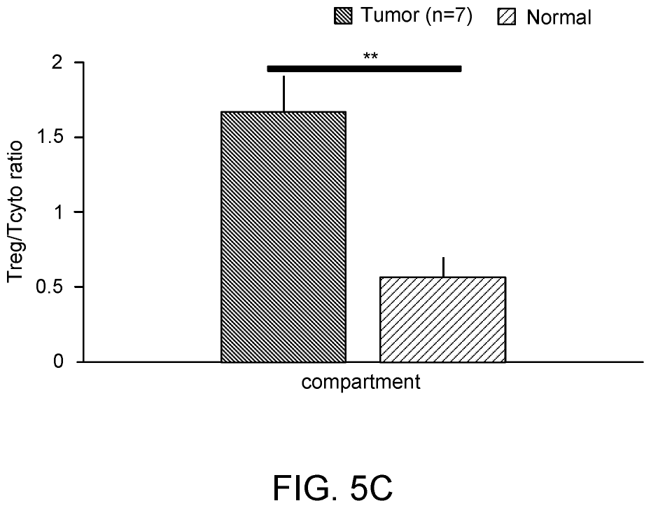

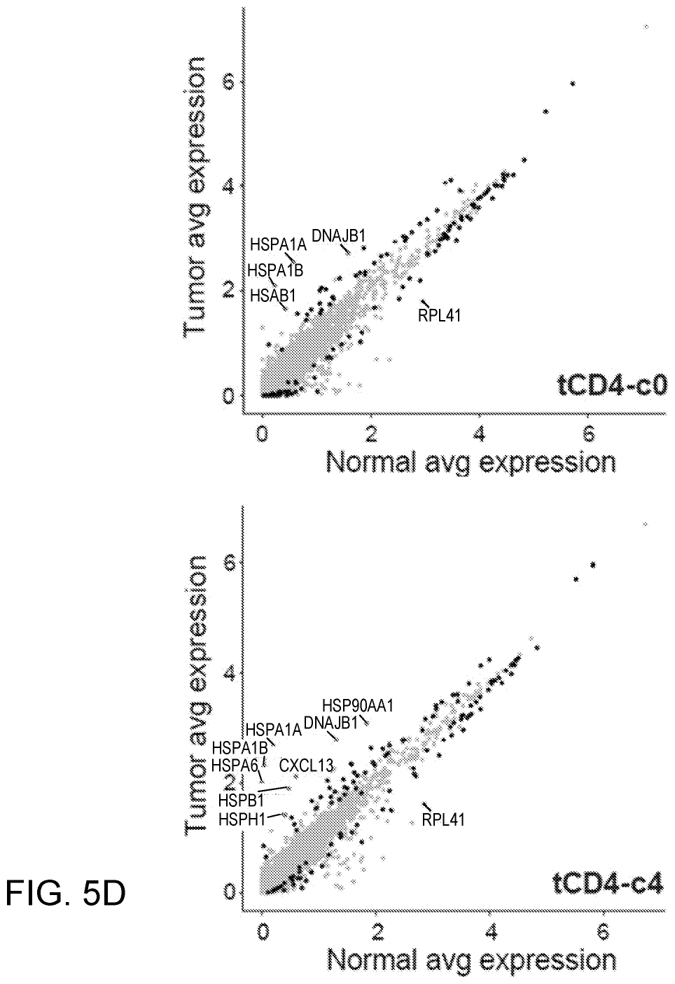

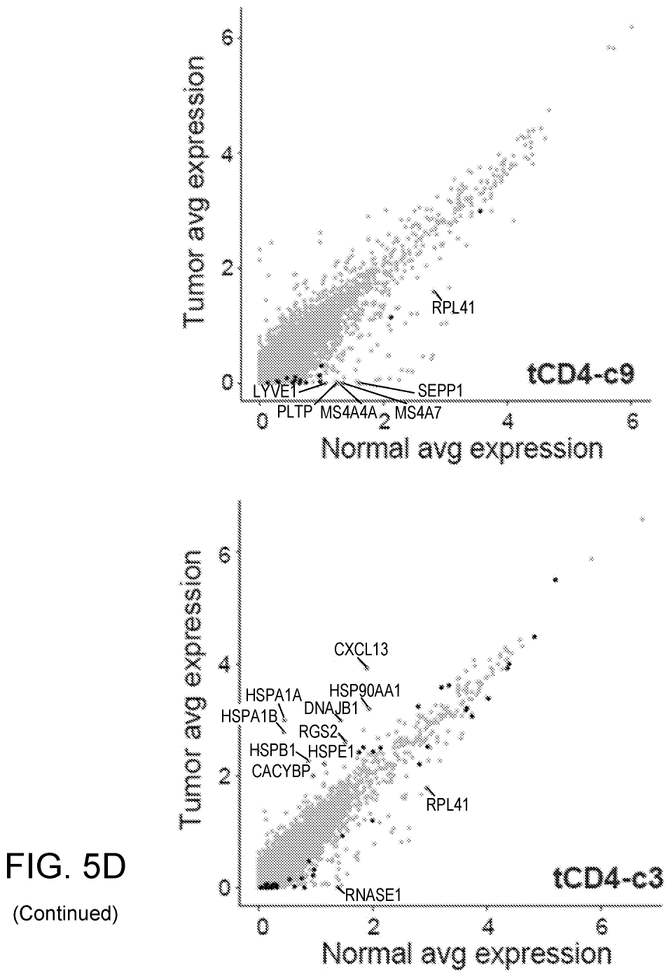

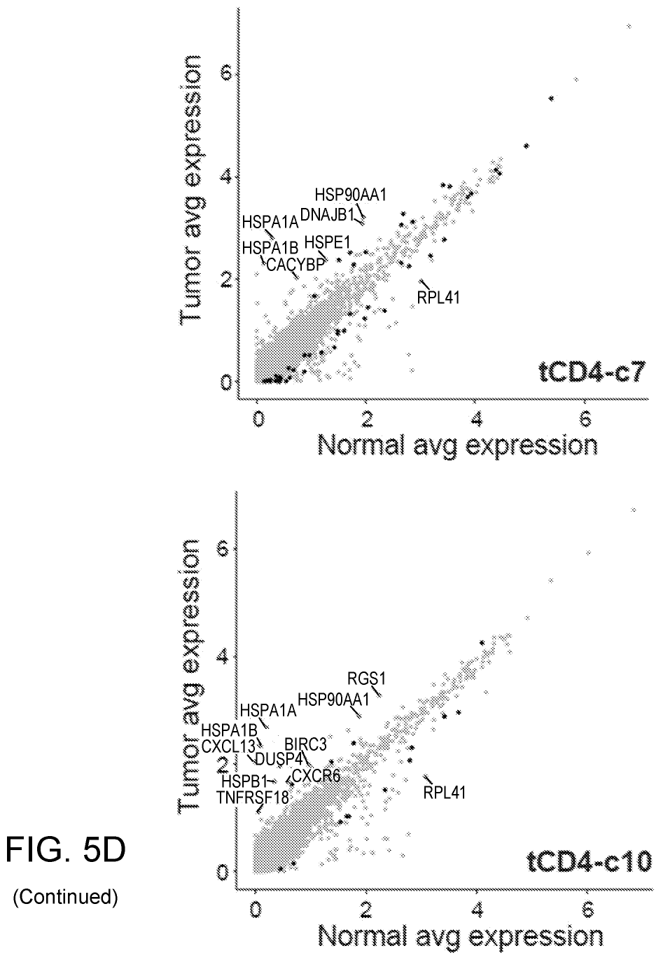

Although each of 14 CD4⁺ populations contain T cells from all patients and compartments, specific populations were differentially enriched in either tumor or non-malignant compartments. When tumor was compared to the non-malignant compartment, it was found that tCD4-c0 (18.0 vs 5.3%, P=7.4×10⁻⁶), tCD4-c5 (9.1 vs 3.5%, P=0.0031), tCD4-c1 (17.8 vs 6.4%, P=0.0025) and tCD4-c6 (8.2 vs 1.4%, P=0.0042) were significantly enriched while tCD4-c2 (3.3 vs 38.9%, P=0.0047), tCD4-c8 (4.9 vs 11.5%, P=0.0044) and tCD4-c13 (1.4 vs 3.9%, P=0.011) were significantly depleted (T-test, FDR<0.1, FIG. 5A). In contrast, it was also found that CD8⁺ populations did not display any significant differences in frequencies between the tumor and non-malignant bladder (FIG. 5B), In addition, CD4⁺ populations also demonstrated increased abundance of regulatory relative to cytotoxic T cells in tumors as compared to non-malignant tissues (regulatory:cytotoxic CD4⁺ ratio=1.7±0.23 in tumor vs 0.6±0.13 in non-malignant, P=0.002 by T-test, FIG. 5C).

Across populations, the tumor-specific T cells expressed a canonical set of genes. For CD4⁺ T cells, multiple populations showed tumor-specific expression of heat shock proteins and immune transcripts such as IL32 (tCD4-c0, -c2, -c5, -c6, -c8, -c13), CXCL13 (tCD4-c2, -c3, -c4, -c8, -c10), and CD3D (tCD4-c1, -c5, -c6,), (all genes with P_{adj}<0.05, |log₂(FC)|>0.5; see, e.g., FIG. 5D). Differential expression analysis for CD8⁺ T cells comparing paired tumor/non-malignant compartments also revealed multiple populations with tumor-specific heat shock expression as well as tumor enrichment of MHC class II alleles including HLA-DRA/-DRB1/-DPA1/-DPB1 and CD74 (Class II-associated invariant chain) which was likely reflective of activation by antigen (all genes with P_{adj}<0.05, |log₂(FC)|>0.5; data not shown).

Hence, without being bound to theory, it is concluded that the bladder tumor microenvironment is characterized by a particular enrichment of regulatory T cells relative to cytotoxic CD4⁺, as well as conserved expression of activation markers or immune checkpoints across several populations that are not observed in the CD8⁺ compartment.

### Example 8

### Intratumoral Cytotoxic CD4⁺ are Clonally Expanded in Bladder Tumors and Modulated by Anti-PD-L1 Therapy

To query the antigenic specificities of the TCR in the same single cells for which whole-transcriptome data was available, the complementarity-determining region 3 (CDR3) sequences of the TCR alpha (TRA) and beta (TRB) loci were PCR-amplified from the barcoded full-length cDNA library and sequenced the resulting library to saturation. After filtering, this approach yielded 12,855 CD4⁺ T cells (58% of cells with expression data) and 7,038 CD8⁺ T cells (57% of cells with expression data) with paired TRA and TRB CDR3 sequences. These results were consistent with the expected frequency based on recovery of individual TRA (CD4⁺: 62%, CD8⁺; 60%) and TRB (CD4⁺: 69%, CD8⁺: 68%) sequences (data not shown).

The TCR repertoire was found more restricted in the tumor microenvironment than adjacent non-malignant tissue. In intratumoral CD4⁺ T cells, 9.9±1.4% of unique paired TRA/TRB clonotypes were shared by 2 or more cells; this degree of sharing was significantly greater than in the non-malignant compartment (4.6±1.4%; unpaired T-test, P=0.021), and was not seen in healthy donors processed using our protocol (0.09-0.23%) or from publicly available reference circulating CD4⁺ data (0%) (FIG. 6A). This was reflected in a skewing of the intratumoral CD4⁺ repertoire towards an increased cumulative frequency of clonotypes over fewer cells (FIG. 6B) and a corresponding higher Gini coefficient (0.19 for tumor vs 0.05 for non-malignant, unpaired T-test, P=0.005, FIG. 6C) compared to the non-malignant compartment and healthy controls. Repertoire restriction was also seen in CD8⁺ T cells from the same samples, but CD8⁺ cells from both tumor and non-malignant compartments demonstrated similarly high percentages of unique clonotypes shared between cells (14±0.97% and 14.3±1.5%, FIG. 6D) and high Gini coefficients (0.34±0.04 and 0.37±0.04, FIGS. 6E-6F). The percentage of shared clonotypes was significantly higher in CD8⁺ than CD4⁺ from tumor (unpaired T-test, P=0.03). Hence while intratumoral repertoire restriction was a general property of T cells in bladder tumors, CD8⁺ demonstrated a further degree of restriction than CD4⁺ within tumors, as well as restriction in the non-malignant compartment that was not seen with CD4⁺ T cells. This was likely due in part to decreased diversity of the CD8⁺ repertoire and decreased CD8⁺ infiltration.

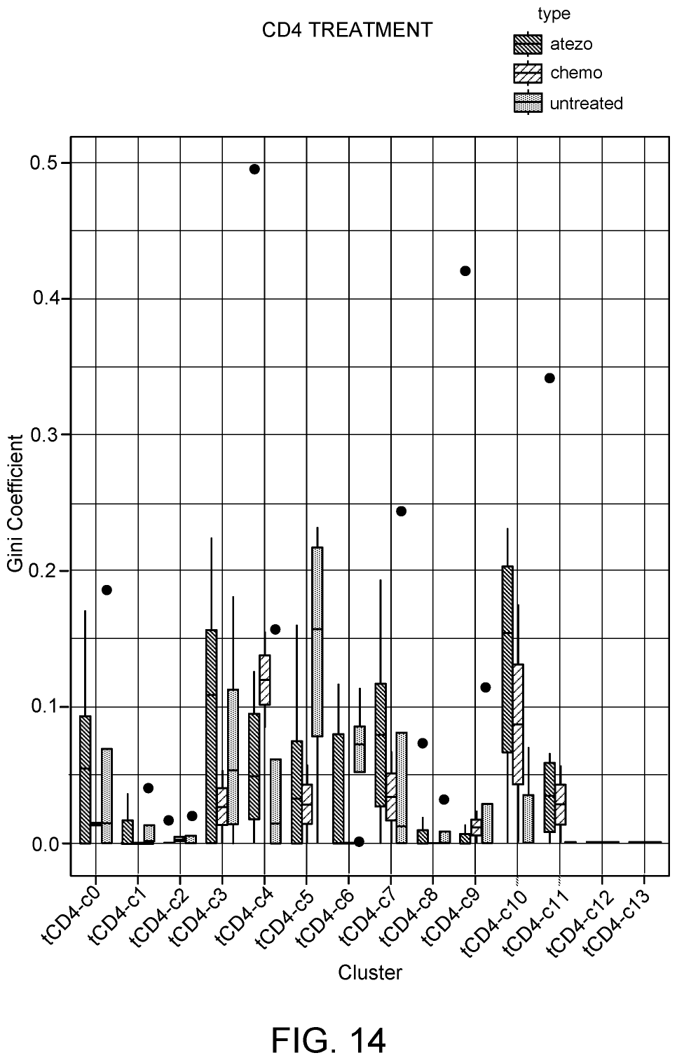

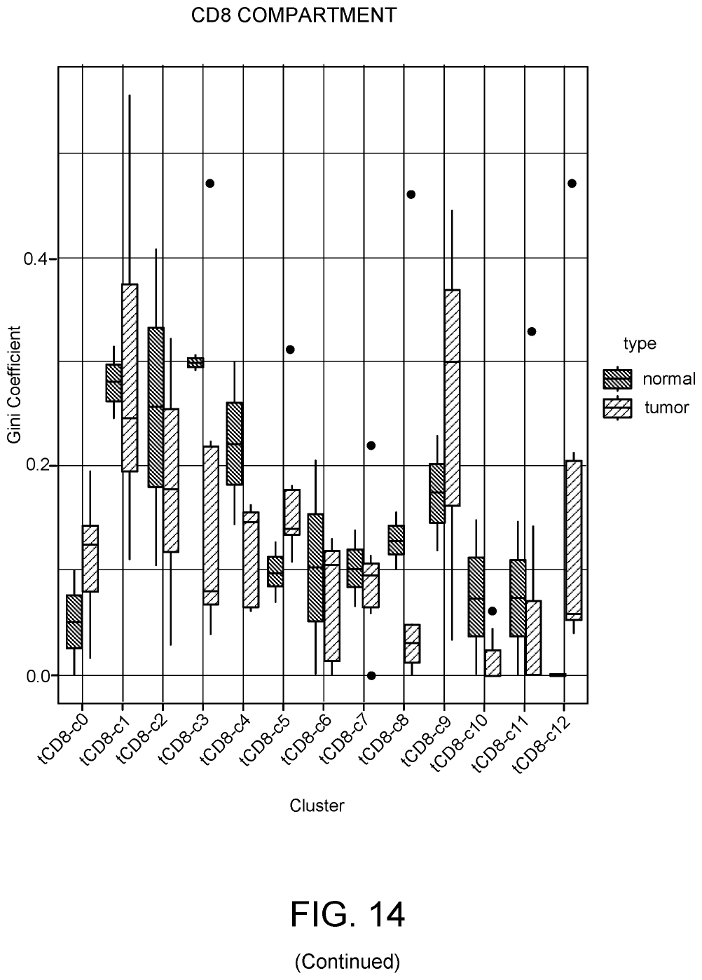

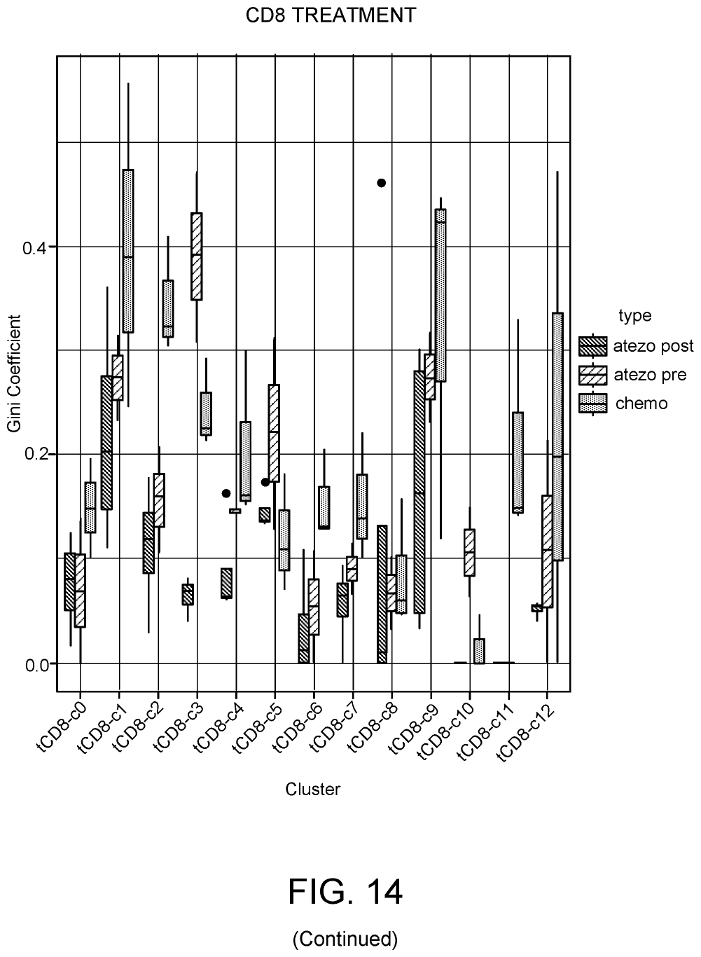

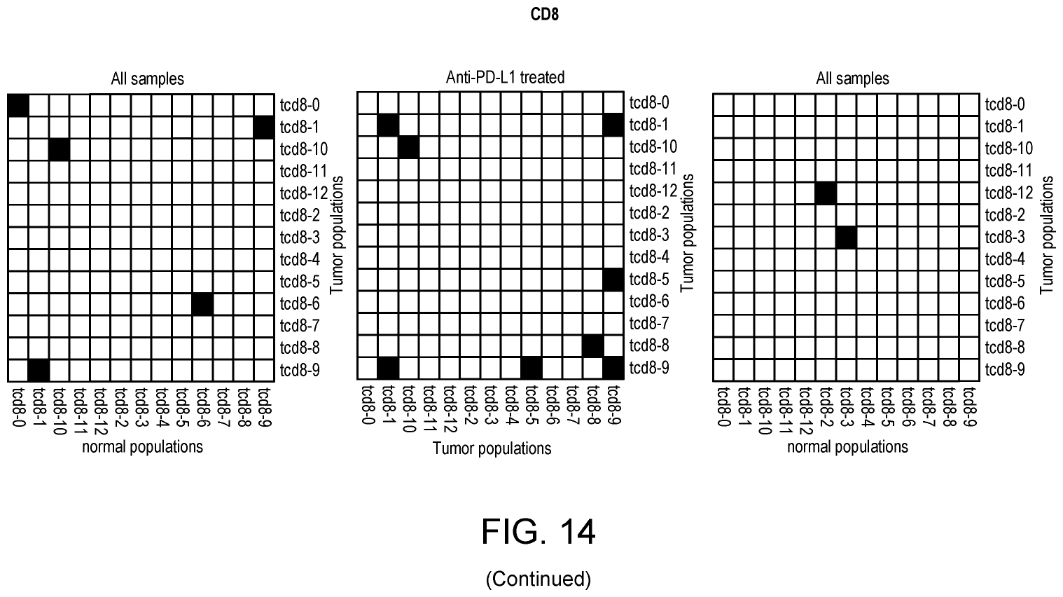

Specific CD4⁺ populations, in particular cytotoxic CD4⁺, demonstrated greater clonal expansion within the tumor. Both regulatory T cell populations (tCD4-c0 and tCD4-c5), tCD4-c3 and 3 cytotoxic CD4⁺ populations (tCD4-c4/c7/c9) exhibited significantly increased repertoire restriction in tumor compared to paired non-malignant tissue. Of these, the most restricted population was the cytotoxic population tCD4-c4 (mean Gini coefficients in tumor versus non-malignant: tCD4-c0 0.09 vs 0.002; tCD4-c3 0.13 vs 0.02; tCD4-c4 0.15 vs 0.02; tCD4-c5 0.12 vs 0.02; tCD4-c7 0.12 vs 0.01; tCD4-c9 0.08 vs 0; P<0.01 for CD4-c0/c3/c7 and P<0.05 for CD4-c4/c5/c9, Wilcoxon test FDR<0.1, FIG. 6G). Tumor-specific clonal expansion was not seen with any CD8⁺ population (see, e.g., FIG. 14). Thus, both regulatory as well as multiple cytotoxic CD4⁺ populations were clonally expanded in the bladder tumor environment.

Analysis of TRA/TRB clonotype sequences that are shared between CD4⁺ cells, and the functional populations to which they belong, identified sharing of the exact same antigenic specificity between functionally related and to a lesser extent functionally distinct populations (FIG. 6H). Quantitative analysis confirmed significant clonotype sharing between cells from the same population within tumor in several cases (tCD4-c0, tCD4-c1, tCD4-c6; empirical permutation P value <0.01, left panel, FIG. 6I). Further, significant intratumoral clonotype sharing was also confirmed between functionally distinct populations, for instance between CXCL13-expressing tCD4-c3 and cytotoxic tCD4-c4 (left panel, FIG. 6I). Within anti-PD-L1-treated samples, although the pattern of sharing was largely similar, anti-PD-L1-specific sharing was seen in tumor within the CXCL13-expressing tCD4-c3 population, and between tCD4-c3 and the functionally distinct cytotoxic population tCD4-c10 (right panel, FIG. 6I). In addition, cytotoxic populations represent the majority of populations sharing clonotypes between paired tumor and non-malignant tissues, either within the same cytotoxic population or among distinct cytotoxic populations (see, e.g., FIG. 14). Similar analyses for CD8⁺ revealed clonotype sharing between tCD8-c1 and activated effector tCD8-c5 in anti-PD-L1-treated samples, and sharing between tumor and non-malignant tissue between pairs of effector tCD8-c3 cells (see, e.g., FIG. 14). Overall, these results highlight that shared antigen recognition in bladder tumors may involve significant plasticity of distinct functional T cell populations. One effect of anti-PD-L1 therapy may be to enhance cytotoxic CD4⁺ recruitment or expansion within tumor leading to clonal expansion and repertoire restriction.

### Example 9

### Cytotoxic CD4⁺ T Cells from Bladder Tumors are Functional

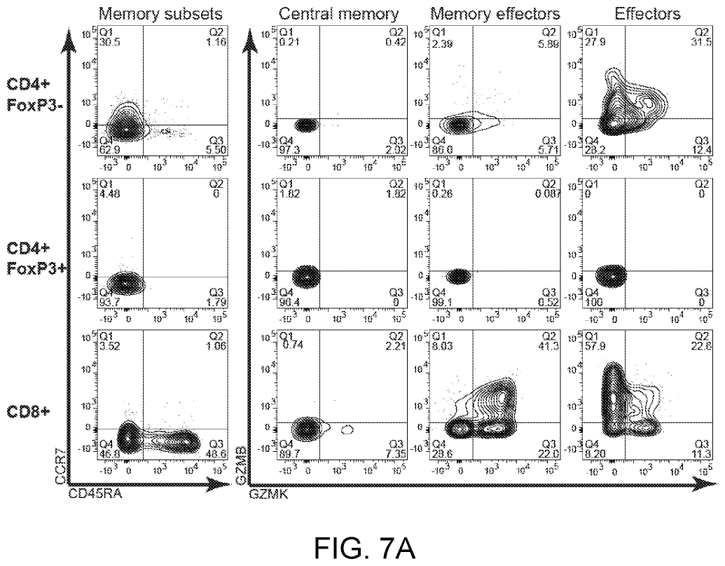

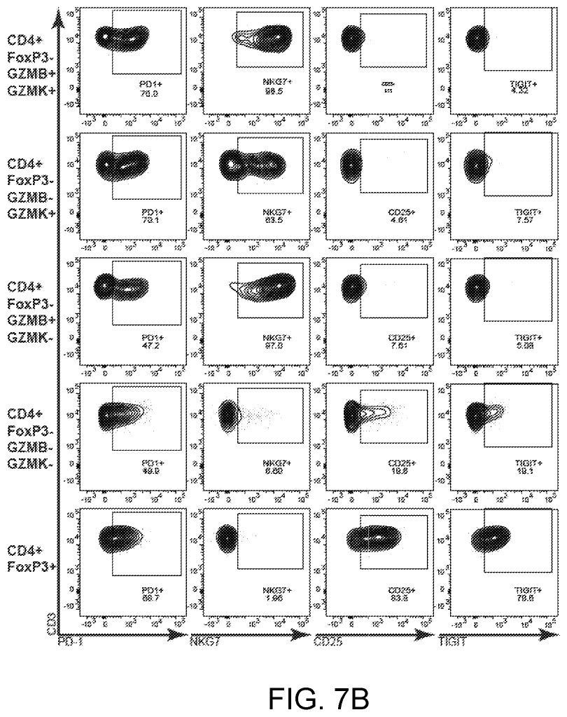

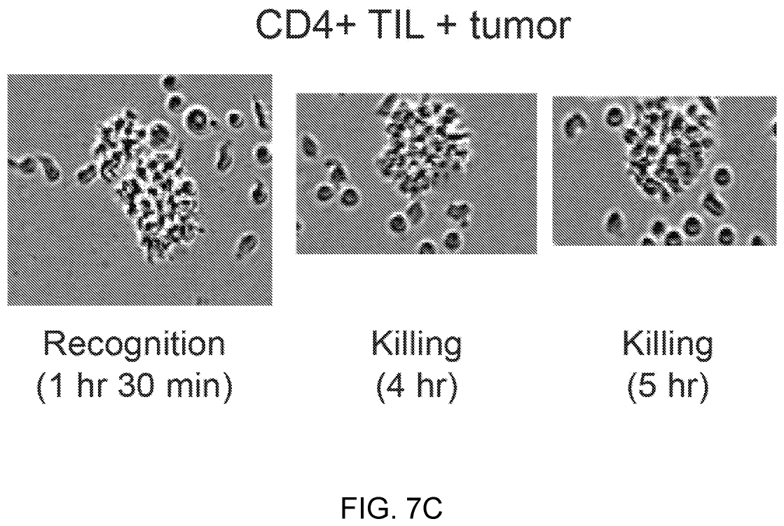

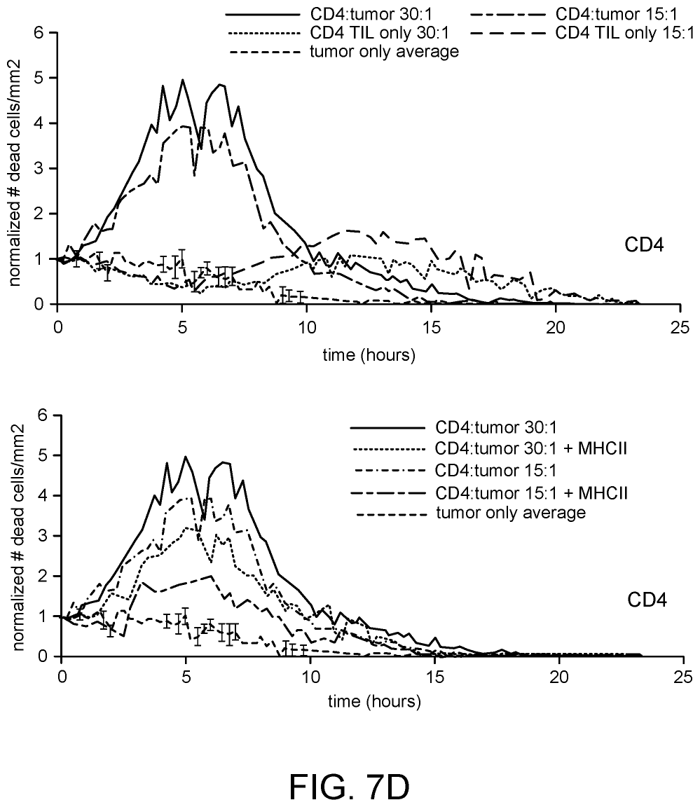

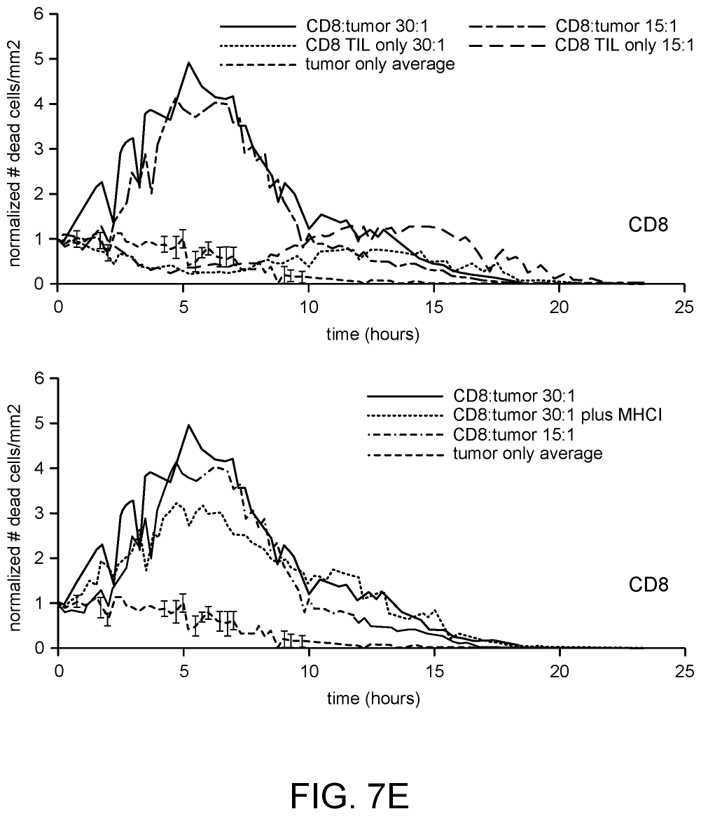

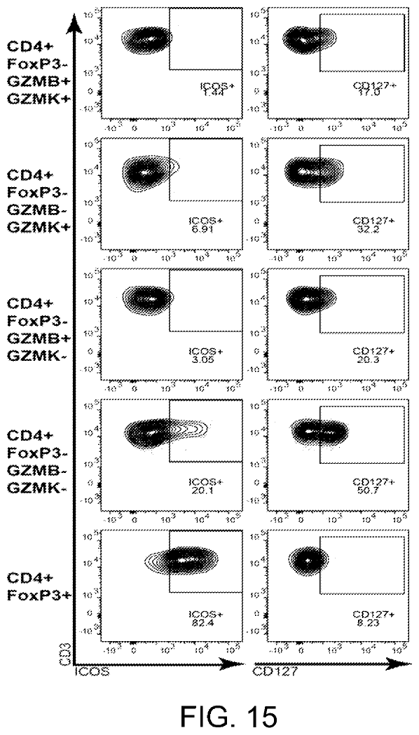

The expression of cytotoxic proteins in CD4⁺ bladder tumor-infiltrating lymphocytes (TIL) was validated by assessing protein expression with flow cytometry analysis. CD4⁺FOXP3⁻ (non-regulatory) cells express combinations of GZMB, GZMK, or both, particularly in the effector population (CCR7⁻CD45RA⁺) and at levels comparable to conventional cytotoxic CD8⁺ from the same patient (FIG. 7A). Of note, the CCR7⁺ cells in the CD4⁺FOXP3⁻ population were lamely CD45RA⁻, consistent with their annotation in the scRNAseq data as central memory rather than naïve (FIG. 7A). Also, while cytotoxic CD4⁺ co-expressed NKG7, they generally did not express checkpoints at high levels such as ICOS/TIGIT or activation markers such as CD25, which were highly expressed in CD4⁺FOXP3⁺ regulatory cells and to a lesser extent in CD4⁺FOXP3⁻GZMB⁻/K⁻ T cells (FIG. 7B and FIG. 15). This was concordant with the scRNAseq data with the exception of low-level expression of CD25 in tCD4-c9 as previously noted. PD-1 expression by flow cytometry, however, was shared across cytotoxic, non-cytotoxic, and regulatory cells in the CD4⁺ compartment (FIG. 7B). Importantly, bladder tumor cells (included in the single viable CD45⁻ gate) expressed multiple alleles of HLA-DR II (data not shown), which would allow for antigen recognition by TCRs expressing CD4 as a co-receptor.

To validate the functional relevance of cytotoxic CD4⁺ in bladder tumors, CD4⁺ TILs were isolated from localized human bladder tumors by FACS, excluding regulatory T cells, and then cultured these ex vivo with IL-2. These cells were then co-cultured with autologous tumor cells in an imaging-based time-lapse cytotoxicity assay. CD4⁺ TILs formed clusters around tumor cells within 1-2 hours of co-culture (indicative of tumor recognition) followed by killing of tumor cells (as measured by an increase in number of cells staining with a red fluorescent cell death indicator) within 4-5 hours (FIG. 7C). An increase in tumor cell death was seen at 5 hours indicative of rapid killing by CD4⁺ TIL (for 30:1 ratio of TIL to tumor, 4.9× increase in death from baseline; FIG. 7D, top panel) that was not seen in surrounding TILs from the same wells containing tumor, or in tumor cells cultured alone in separate wells (TIL only at 30:1: 0.42×, tumor only: 0.99×). The kinetics and extent of autologous CD4⁺ killing are similar to CD8⁺ killing (CD8⁺ at 30:1 TIL to tumor ratio: 4.9× at 5.25 hrs; FIG. 7E). CD4⁺ killing was dose dependent across various effector:target ratios (30:1 ratio: 4.9×, 15:1 ratio: 3.9× at 5 hrs; FIG. 7D, bottom panel) and was also partially blocked by pre-incubation of tumor cells with a pan-MHCII antibody (30:1 ratio: 3.2× at 5 hrs, 15:1 ratio: 1.95× at 6 hrs FIG. 7D). CD8⁺ autologous killing was also similarly blocked in part by MHCI blockade (30:1 ratio: 3.1× at 5 hrs; FIG. 7E, bottom panel). The observation of autologous tumor killing within hours by CD4⁺ and CD8⁺ TILs above the background level of spontaneous death of TILs from the same wells was representative of 2 independent experiments involving distinct aliquots from the same patient. Hence, flow cytometry and functional analyses confirmed that cytotoxic CD4⁺ not only express cytolytic proteins such as granzymes, but that these cells were functionally competent to lyse tumor cells in an MHC-II-dependent fashion.

### Example 11

### A Subset of Tumor-Infiltrating Cytotoxic CD4⁺ Circulate

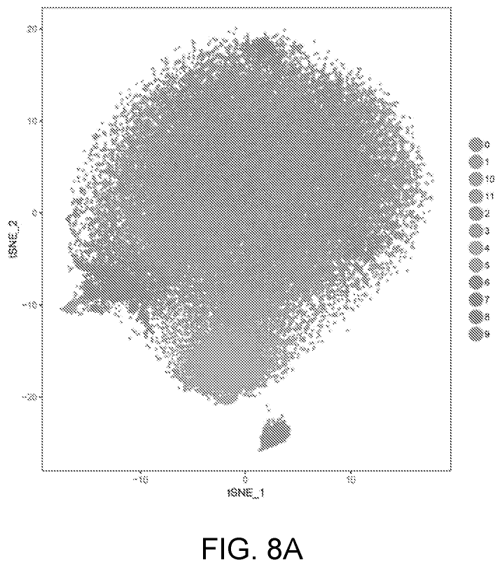

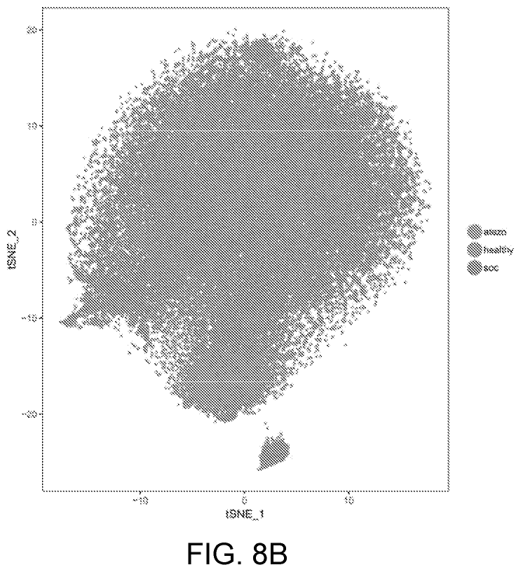

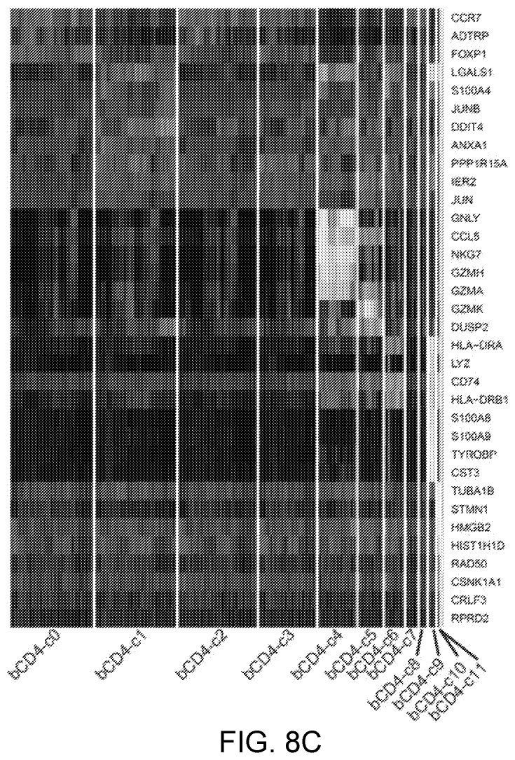

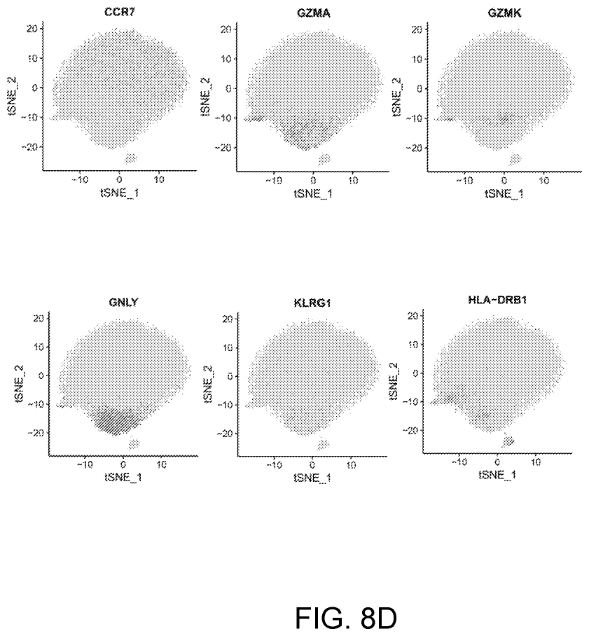

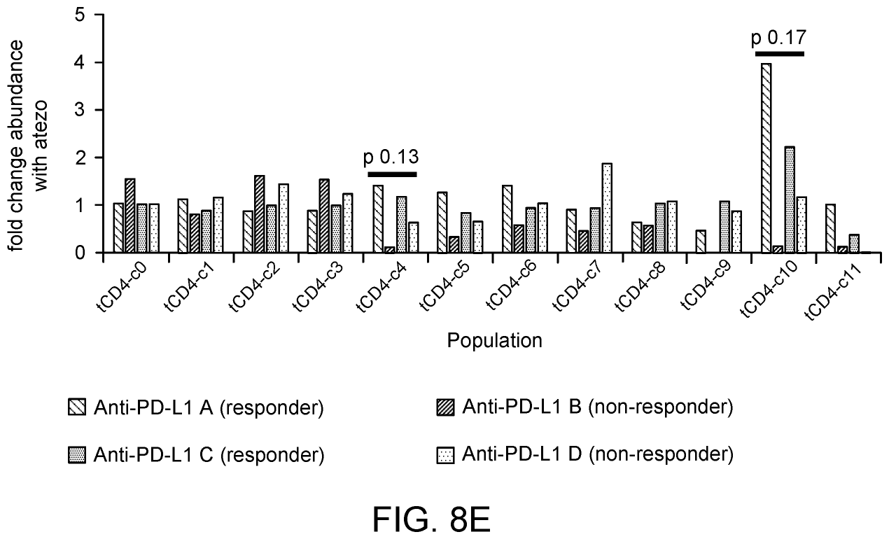

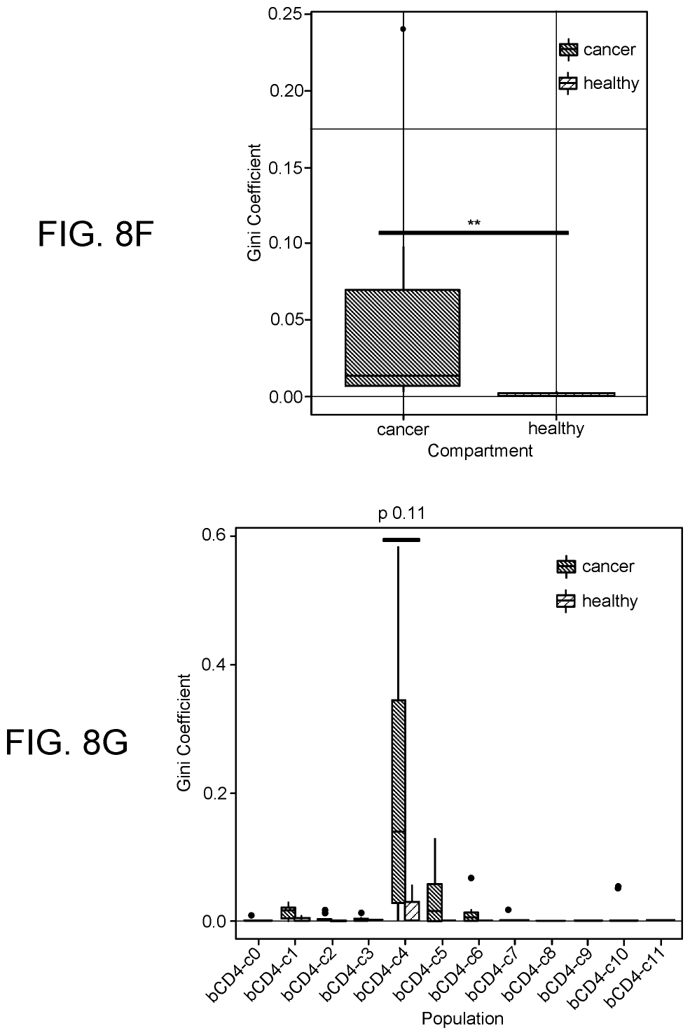

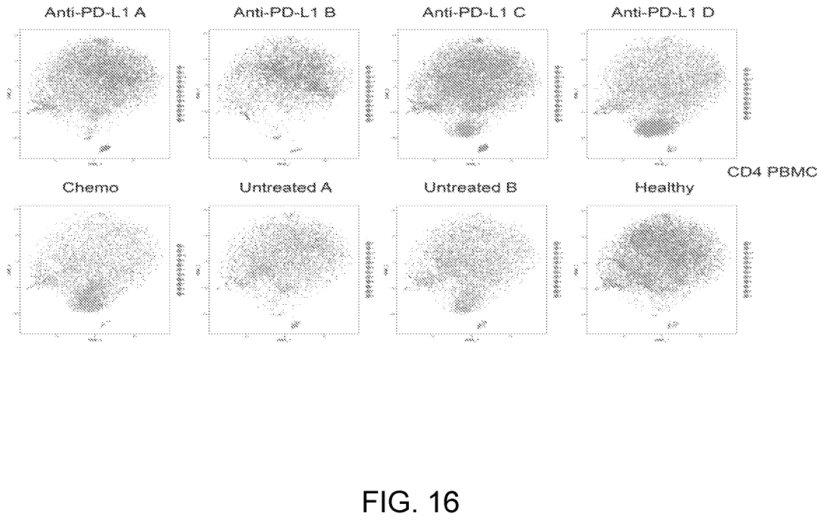

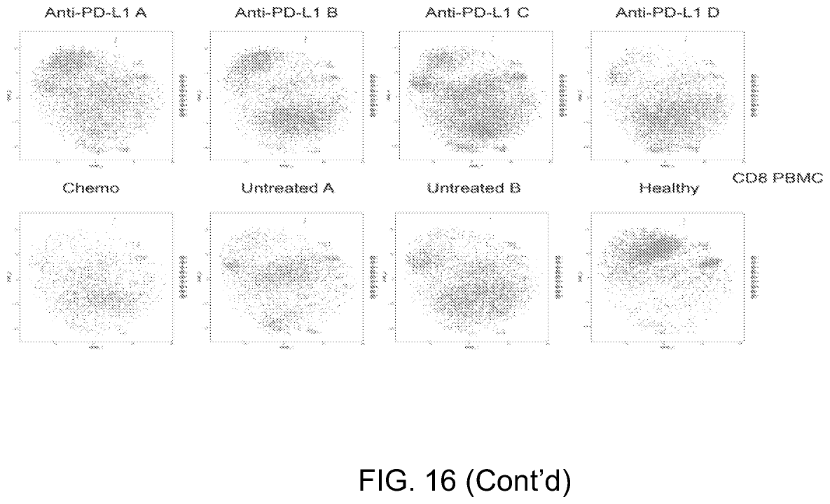

To understand systemic T cell responses in bladder cancer and how these relate to intratumoral responses, we also sequenced circulating T cells from the blood of the same patients. For sorted CD4⁺, this encompassed 75,016 cells from 7 bladder cancer patients and one healthy control patient (data not shown). Of 12 conserved CD4⁺ populations identified by unbiased clustering of the samples above, which were represented in all patients (FIG. 16), notably we identified 2 cytotoxic populations which together included 15±3.1% of all cells (FIGS. 8A-8D). The first cytotoxic population expressed high levels of GNLY, NKG7, GZMH, GZMB, PRF1, and KLRG1 (bCD4-c4: log₂(FC)=3.6, 3.4, 2.8, 1.7, 1.4, 1.2), while the second expressed GZMK and KLRB1 (bCD4-c5: log₂(FC)=2.3, 0.83), Compared to a reference CCR7⁺ population bCD4-c0, both cytotoxic populations expressed GZMA (log₂(FC)=1.8-2.5) (all genes with P_{adj}<0.05, FIGS. 8C-8D). Thus cytotoxic CD4⁺ are also a feature of the circulating immune compartment in localized bladder cancer.

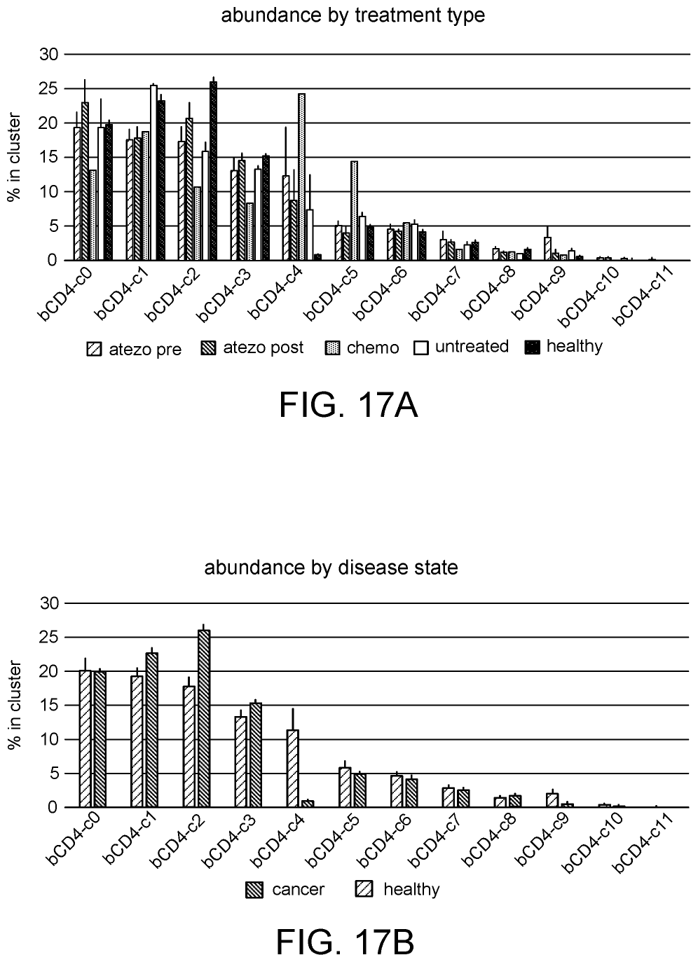

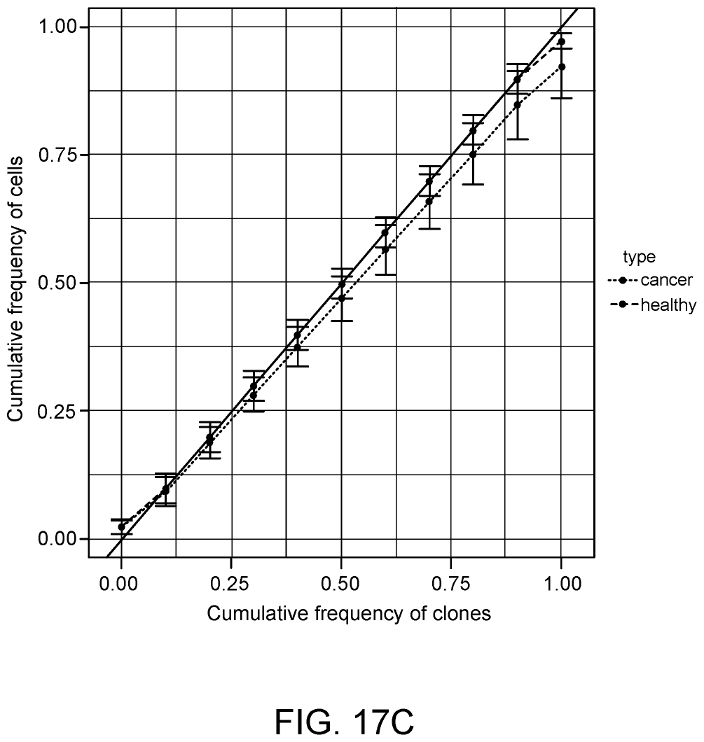

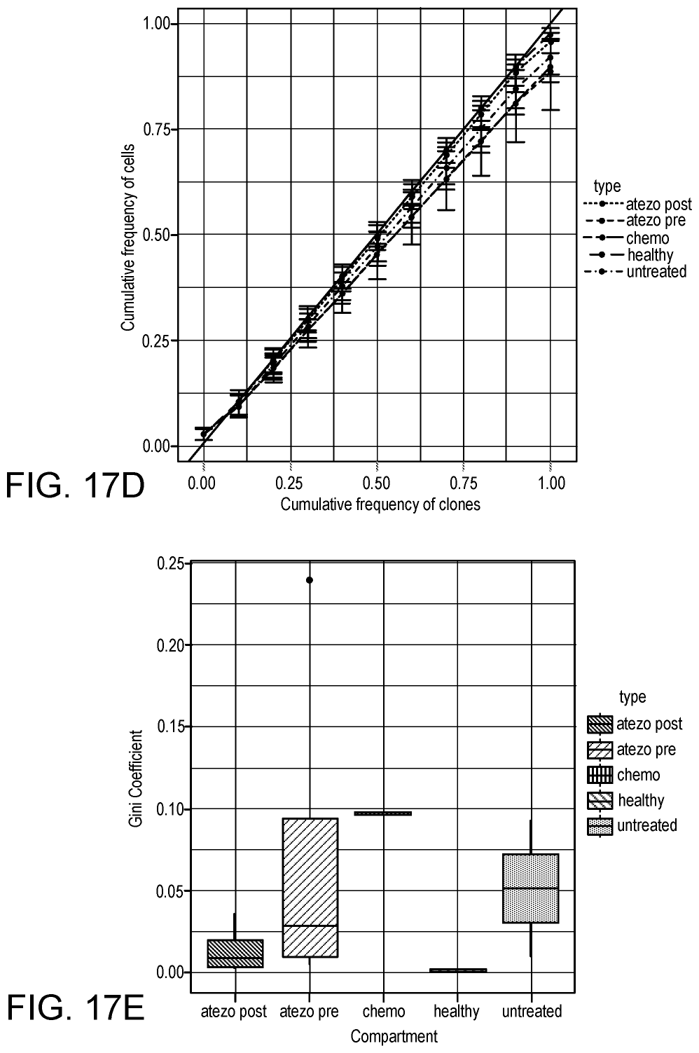

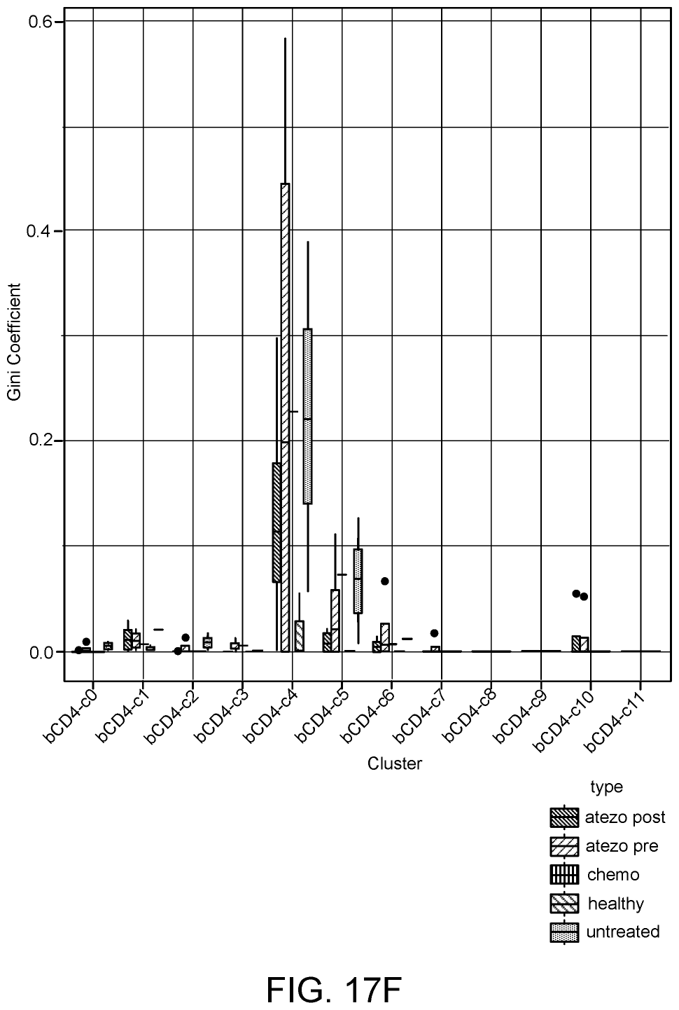

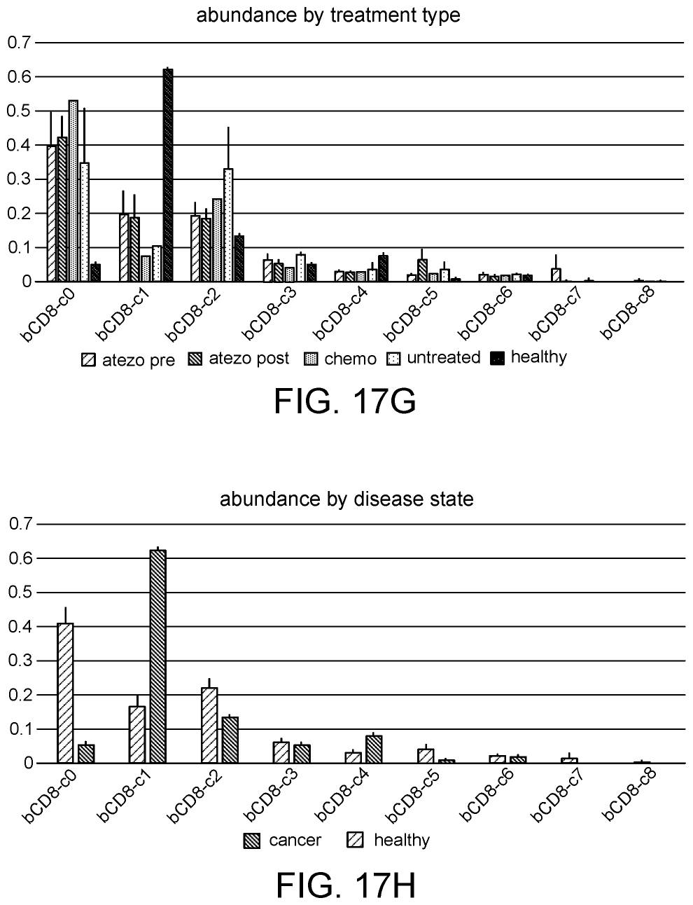

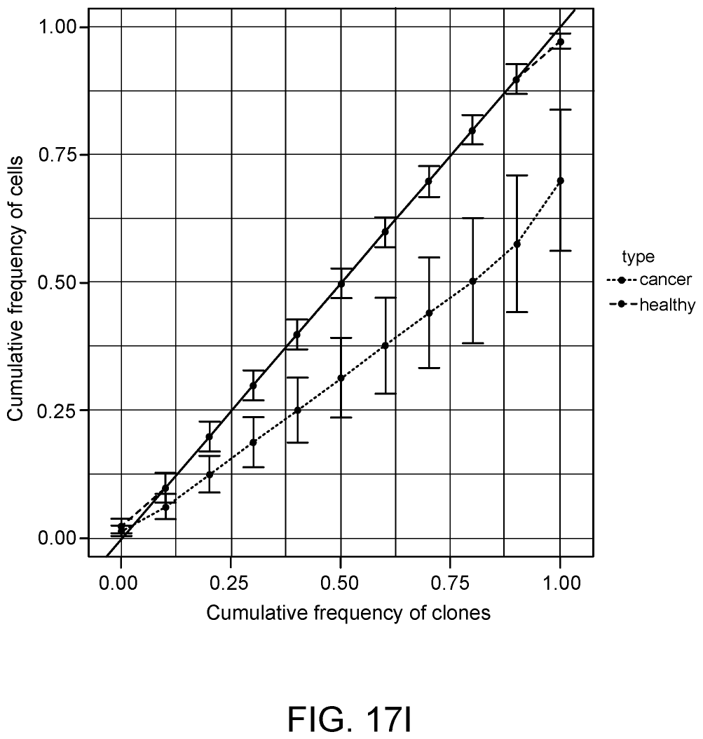

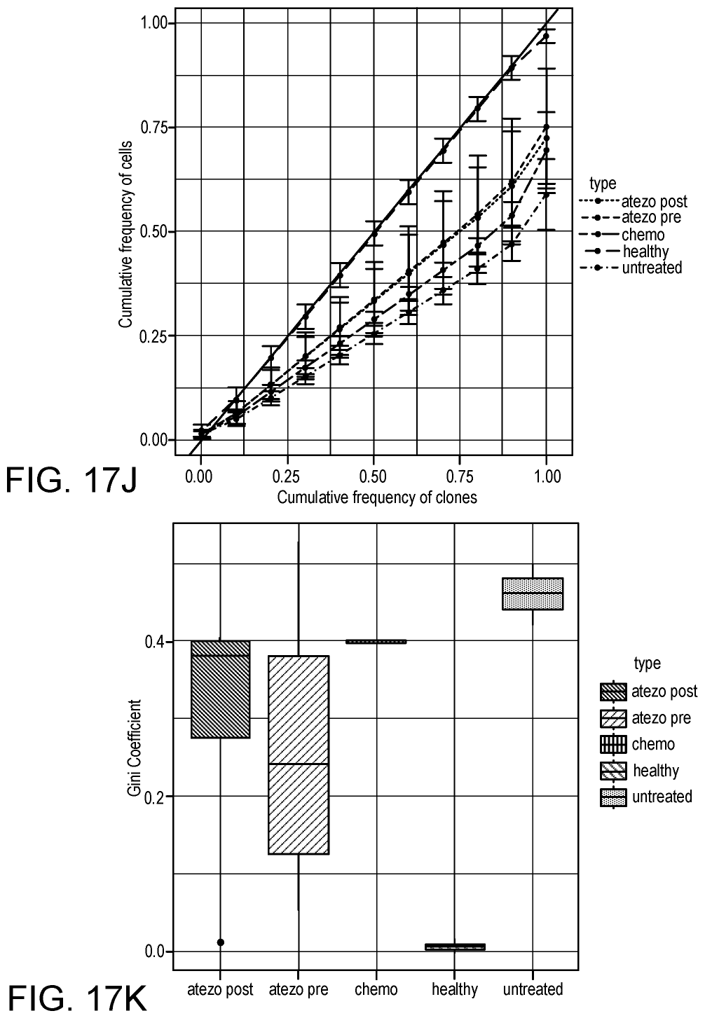

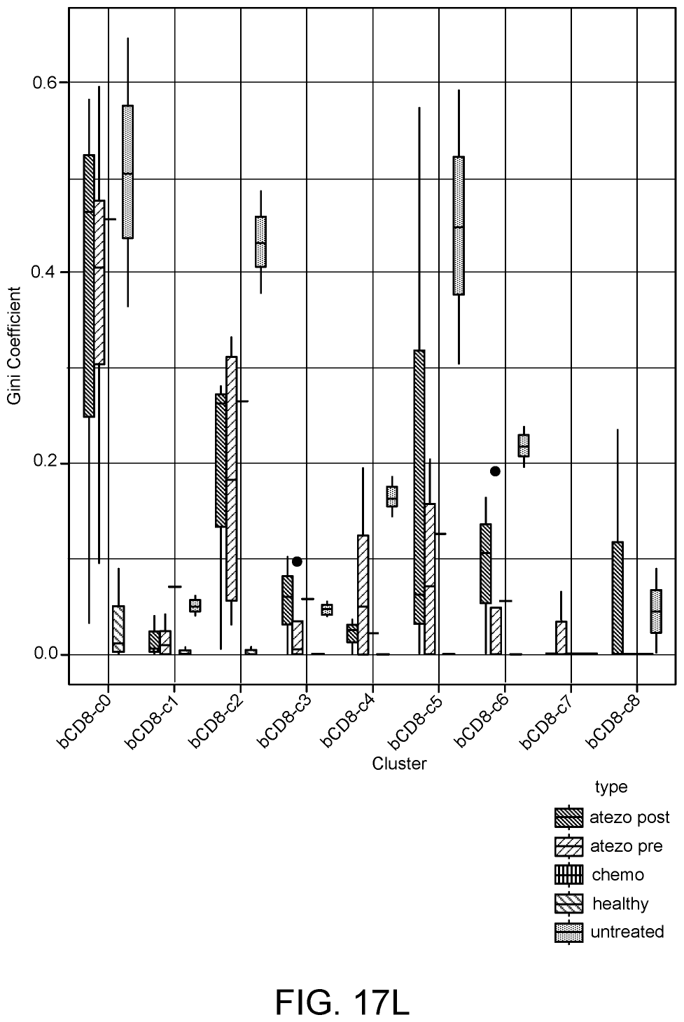

Repertoire restriction is also a feature of circulating CD4⁺ T cells in bladder cancer. TCR sequencing of the same circulating CD4⁺ T cells yielded paired TCRα/β sequences from 33,656 cells (45% of cells with expression data), in line with expected results based on TRA recovery (53%) and TRB recovery (61%) (data not shown). Cancer patients in general demonstrated a significantly higher Gini coefficient compared to healthy controls (mean Gini coefficient: 0.05 versus 0.01, n=11 samples from 7 bladder cancer patients versus triplicate samples from one healthy donor and a single reference sample from 10× Genomics healthy donor TCR data, FIG. 8F). Gini coefficient within individual populations is not significantly increased or impacted by anti-PD-L1 treatment (FIGS. 17D-17F).

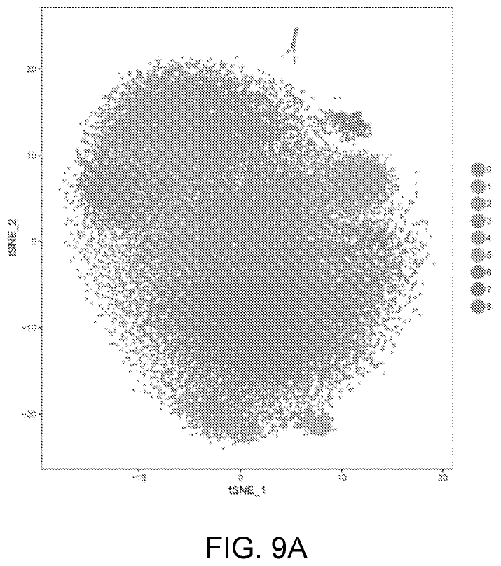

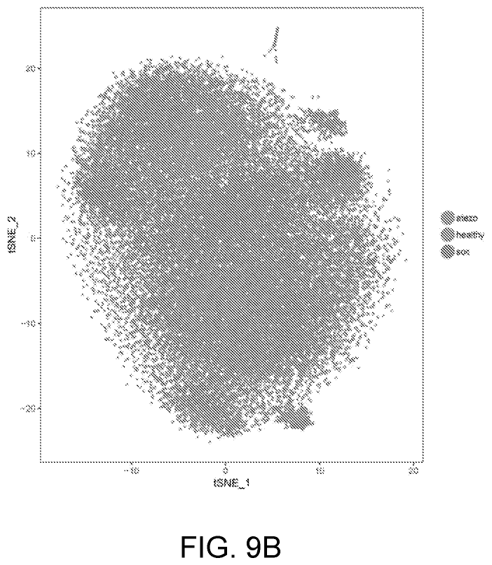

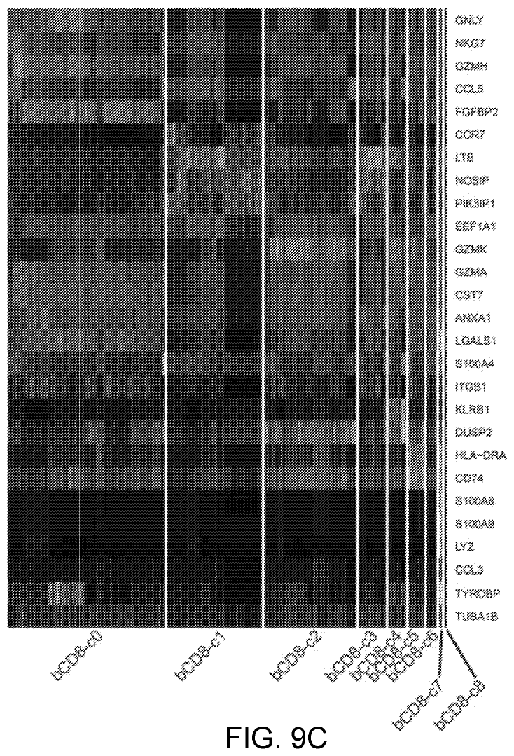

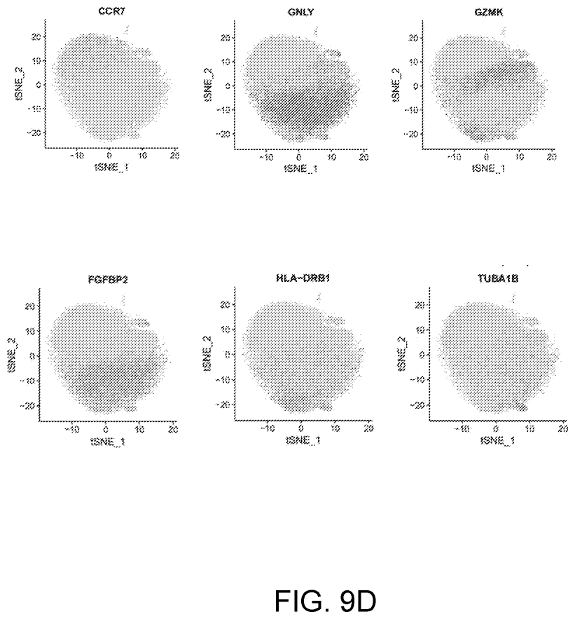

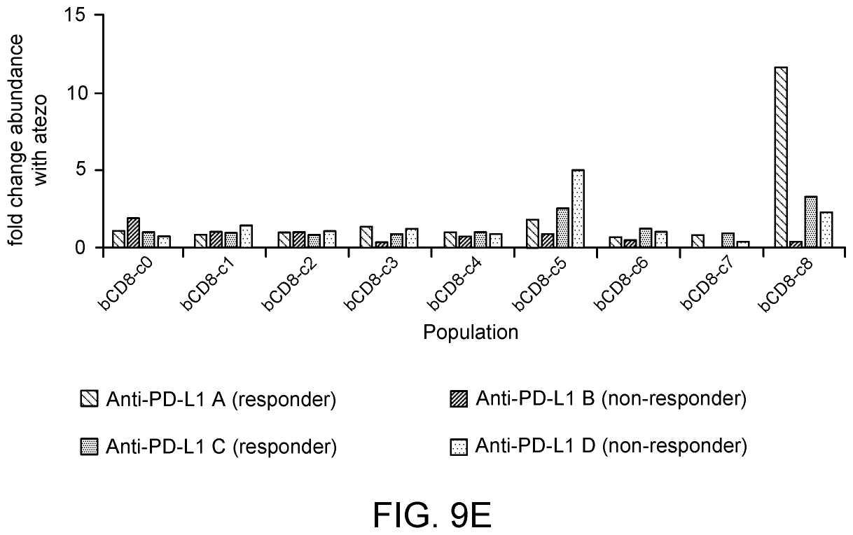

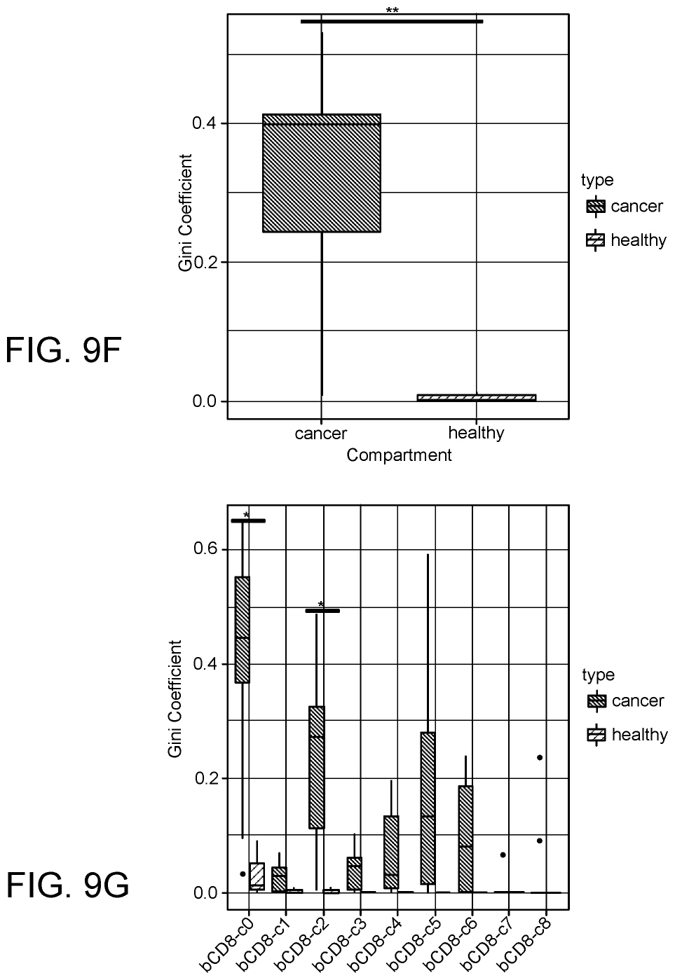

Similar approaches for circulating sorted CD8⁺ were used to identify 9 conserved populations, some of which were specifically clonally expanded. In total we sequenced 50,421 total CD8⁺ from blood from 7 bladder cancer patients and one healthy control patient (Table 1). This reveals a CCR7⁺ population (bCD8-c1), several effector populations including those overexpressing GNLY (bCD8-c0), GZMK (bCD8-c2), NKG7 (bCD8-c6), or MHC II (bCD8-c5), as well as KLRB1-expressing MAIT cells (bCD8-c4) which are enriched for TRAV1-2/TRAJ33 expression (see below) and cycling cells (bCD8-c8) (all genes with P_{adj}<0.05, log₂(FC) versus CCR7⁺ bCD8-c1 reference population, FIGS. 9A-9D). Significant changes were not seen in the absolute abundance of individual populations or relative induction in abundance with anti-PD-L1 (FIGS. 9E and 17G-17H). However, TCR analysis from the same cells which recovered paired TCRα/β from 24,912 cells (49% of cells with expression data; single TRA recovery 59%, single TRB recovery 63%; data not shown), demonstrates that similar to circulating CD4⁺, circulating CD8⁺ also display increased repertoire restriction in cancer patients compared to healthy controls (mean Gini coefficient: 0.32 vs 0.01, n=11 samples from 7 bladder cancer patients versus triplicate samples from one healthy donor and a single reference sample from 10× Genomics healthy donor TCR data, FIG. 9F). This is driven by specific CD8⁺ effectors which demonstrate cancer-specific clonal expansion (mean Gini for cancer versus healthy: bCD8-c0=0.40 versus 0.03, P 0.01; bCD8-c2: 0.24 versus 0.002, P 0.02; T-test FDR<0.1. FIG. 9G). Hence the circulating CD8⁺ compartment in bladder cancer is characterized by clonal expansion of specific effector subsets.

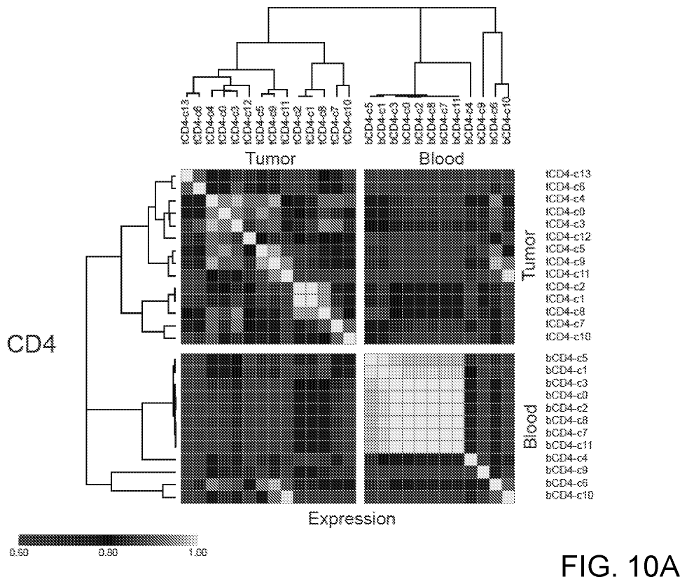

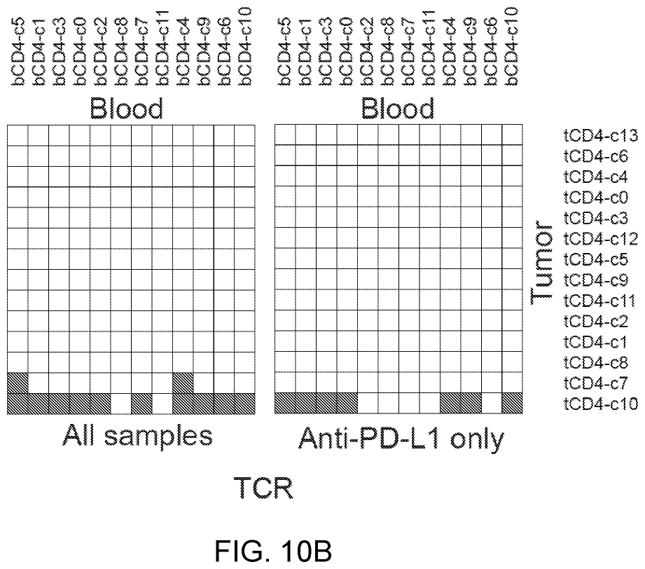

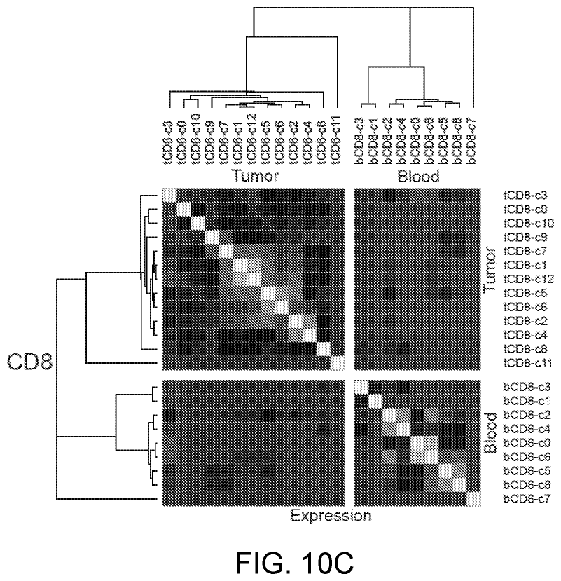

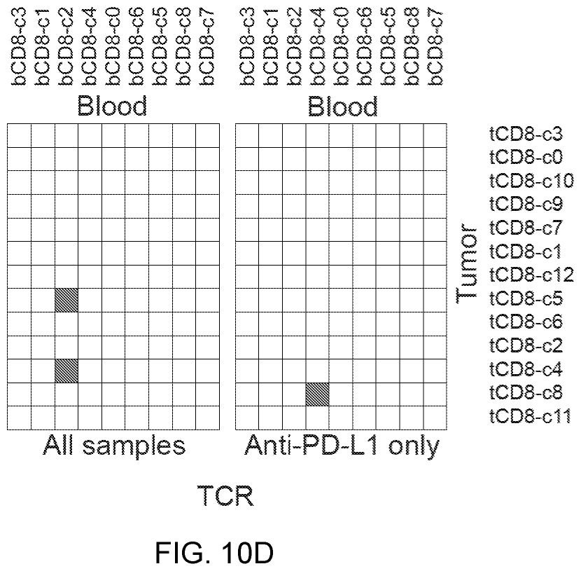

Comparison of CD4⁺ populations from blood and tissue revealed that a subset of cytotoxic CD4⁺ is closely related between these compartments. Correlation of transcriptional profiles revealed close relationships between blood bCD4-c6 effectors and tumor tCD4-c9 cytotoxic cells (R=0.95), as well as between cycling cells in both blood and tumor (R=0.98). As for cytotoxic CD4⁺, the circulating cytotoxic bCD4-c4 were most correlated with intratumoral tCD4-c10 (0.85), while circulating cytotoxic bCD4-c5 were most correlated with intratumoral cytotoxic tCD4-c7 (0.81) as well as several other populations including activated effector tCD4-c8 (0.85) and central memory populations tCD4-c1/c2 (0.81 and 0.82) (FIG. 10A). Examination of TCRα/β clonotypic sharing between blood and tumor populations confirmed that cytotoxic CD4⁺ in blood (bCD4-c4 and c5) are not only functionally related to specific intratumoral cytotoxic CD4⁺ (tCD4-c7 and -c10), but also share the exact same antigenic specificity to a significant degree (FIG. 10B, left panel). Of note, the intratumoral NKG7⁺ tCD4-c10 population also shares clonotypes with other non-cytotoxic populations in the periphery including several CCR7⁻ overexpressing populations, and activated/cycling cells (bCD4-c6, bCD4-c10), underscoring plasticity in the phenotype of cells that share specificity between compartments (FIG. 10B, left panel). Intercompartment clonotype sharing in the subset of anti-PD-L1-treated samples involves cytotoxic CD4⁺ in blood and tumor, but is restricted to the intratumoral cytotoxic tCD4-c10 population (FIG. 10B, right panel). Thus, specific subsets of cytotoxic CD4⁺ in the blood and tumor constitute a major class of CD4⁺ effectors that share both specificity and function between these compartments. In addition, intratumoral cytotoxic CD4⁺ can also share specificity with functionally distinct phenotypes in the periphery. Similar approaches for CD8⁺ populations demonstrated significant sharing of specificity between populations in blood and tumor that are moderately correlated by function (circulating cytotoxic bCD8-c2 and intratumoral activated tCD8-c5, R=0.84 in all samples), as well as clonotype sharing between functionally related KLRB1⁺ populations in blood and tumor that is only seen in the anti-PD-L1-treated samples (R=0.86) (FIGS. 10C-10D). As this latter population is thought to represent mucosal-associated invariant T cells (MATT), this may reflect enhanced sharing of antigenic specificity between these cells in both tumor and periphery in response to PD-1 blockade.

### Example 12

### Distinct Phenotypic Classes of Cytotoxic CD4⁺ Circulate in the Periphery of Bladder Cancer Patients, and May be Selectively Modulated by PD-1 Blockade

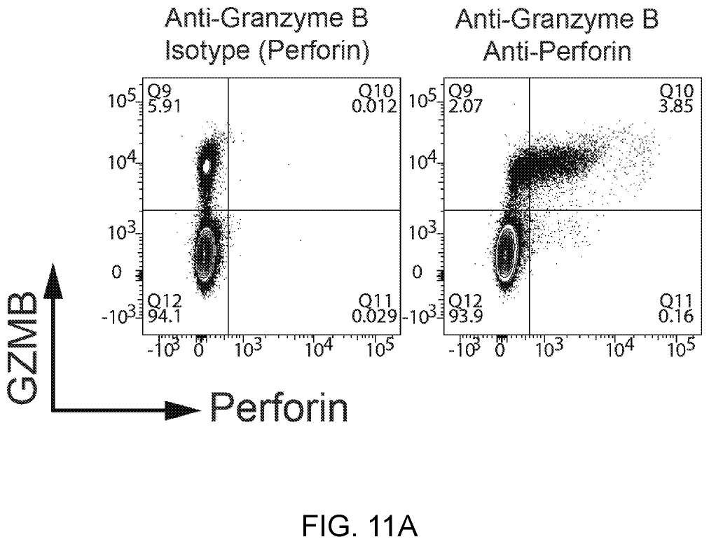

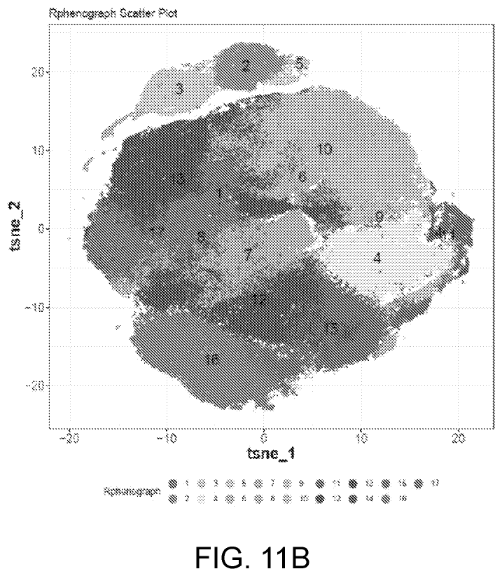

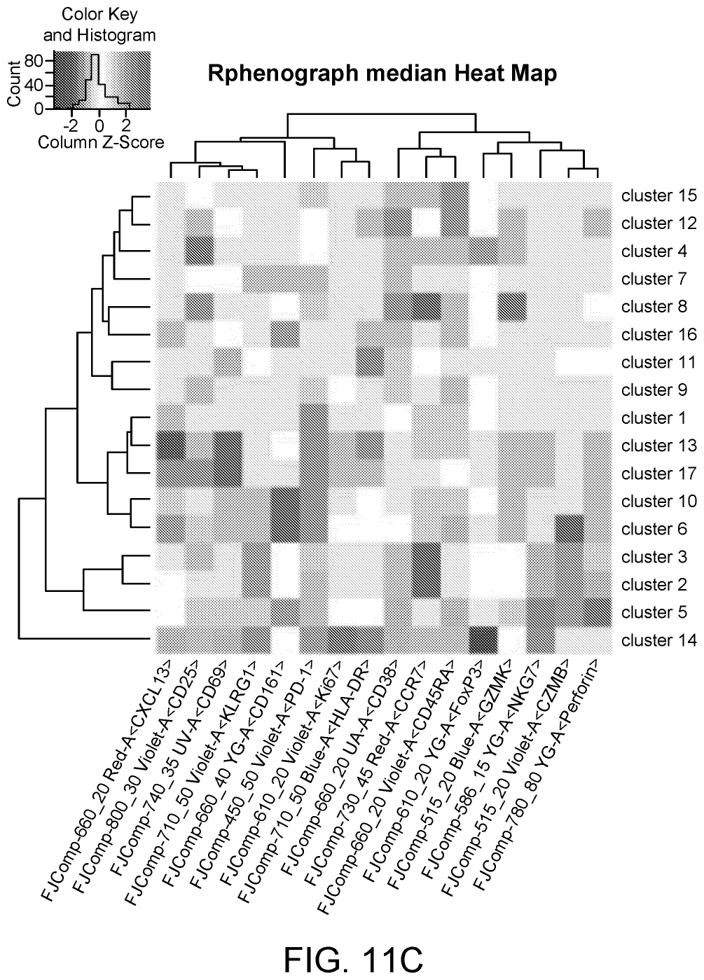

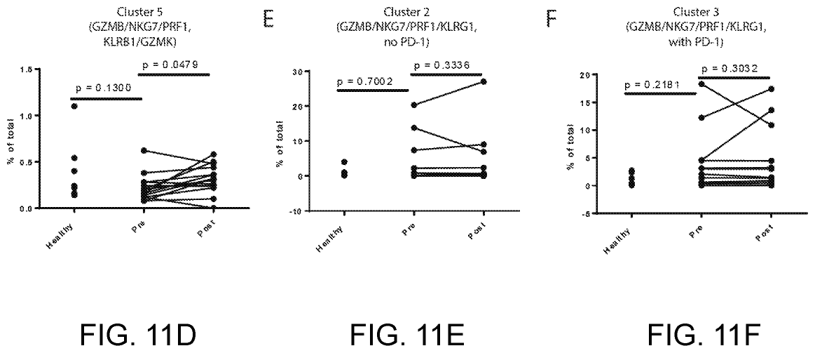

Flow cytometric analysis of PBMCs before and after treatment from all 14 patients treated with anti-PD-L1 therapy on this neoadjuvant trial (including the 4 patients for whom we obtained scRNAseq data), in conjunction with PBMCs from 8 healthy control patients, verifies that bona fide cytotoxic CD4⁺ circulate in bladder cancer patients based on protein level expression of GZMB and PRF1 (FIG. 11A). Using Phenograph (ref) to identify communities of related populations in this flow cytometric data (FIG. 11B), we can identify several distinct subclasses of cytotoxic CD4⁺ in the periphery of these patients, including populations strongly expressing GZMK (Cluster 8), several populations co-expressing GZMB/PRF1/NKG7/KLRG1 either with PD-1 (Cluster 3) or without PD-1 (Cluster 2), and finally a population that expresses both GZMB/PRF1/NKG7 as well as GZMK and KLRB1 (Cluster 5) (FIG. 11C). This validates the circulating cytotoxic CD4⁺ populations found by dscRNAseq, in particular Cluster 2 which resembles bCD4-c4, and Cluster 5 which resembles bCD4-c5. Of note, Cluster 5 demonstrates induction in response to anti-PD-L1 treatment in paired samples (P<0.05, FIG. 11D), while Clusters 2 and 3 do not (FIGS. 11E-11F). Hence phenotypic variation in cytotoxic CD4⁺ appears to be functionally relevant, as specific circulating subsets found in bladder cancer patients may be more responsive to PD-1 blockade.

Taken together, the experimental data presented above demonstrate that unbiased massively parallel genotypic and phenotypic profiling of the T cell compartment in localized bladder tumors and the adjacent non-malignant compartment, including those treated with anti-PD-L1 immunotherapy, to can be used to demonstrate several key conceptual advances in our understanding of tumor immunity.

First, in the context of localized muscle-invasive bladder cancer, the experimental data presented herein showed that tumor-infiltrating T cells favor CD4⁺ infiltration over a relative paucity of CD8⁺, and within the CD4⁺ compartment, while regulatory T cells are the predominant immune subtype, cytotoxic CD4⁺ T cells represent a significant component of tumor-infiltrating T cells. These are distinct populations based on both scRNAseq and flow cytometric validation. The annotation using SingleR indicates that novel effector populations such as cytotoxic CD4⁺ found in the tumor microenvironment may not yet be annotated, and based on “best-fit” comparisons to external reference data and transcriptional correlation within our own data these cells are in fact most similar to conventional cytotoxic CD8⁺ T cells. While cytotoxic CD4⁺ T cells have been described in non-small cell lung and hepatocellular carcinoma have been shown in the circulation to mediate antigen-specific killing following ipilimumab treatment in metastatic melanoma, and also are found in an infectious context where they represent a clonally expanded dengue virus-specific effector subset, the extent of their heterogeneity in other solid tumors (including bladder cancer), whether cytotoxic CD4⁺ in the tumor-infiltrating lymphocyte compartment are part of a larger systemic anti-tumor response, and whether these cells are modulated by systemic immunotherapy have remained unclear. Several lines of evidence supporting the importance of cytotoxic CD4⁺ as anti-tumor effectors were found. Several discrete populations of cytotoxic CD4⁺ were clonally expanded in tumor compared to non-malignant tissue, suggesting recognition of cognate antigens within the tumor microenvironment. Moreover, cytotoxic CD4⁺ were found to be part of a systemic immune response to muscle-invasive bladder cancer, as despite the presence of only localized disease, cytotoxic CD4⁺ were also found in the circulation of these patients, and specific subsets of intratumoral and circulating cytotoxic CD4⁺ that expressed GZMB, NKG7, and GZMK are functionally related and represented the major class of CD4⁺ that shared exact antigenic specificity between these compartments. This underscores the importance of cytotoxic CD4⁺ heterogeneity and suggests that this specific cytotoxic subset may represent a coordinated response across compartments to conserved bladder tumor antigens. It was also found that intratumoral cytotoxic CD4⁺ also shared specificity with functionally discrete non-cytotoxic populations within tumor and in the circulation, indicating a degree of functional plasticity of T cells in the bladder cancer, which has also been described in other contexts. Finally, the relevance of the cytotoxic CD4⁺ populations as a bona fide effector population can be demonstrated by validation of their expression of cytolytic effector proteins in tumor and blood, and by the functional capacity of cytotoxic CD4⁺ TIL to kill autologous tumor cells. These findings were seen in the context of minimal enrichment in tumor or clonal expansion of CD8⁺ population under the same conditions from the same samples. These findings established a paradigm of cytotoxic CD4⁺ as an additional and important means by which PD-1 blockade can generate protective anti-cancer immunity in both the tumor and circulation of cancer patients.

This data presented herein raises several intriguing possibilities. Without being hound to any particular theory, the presence of cytotoxic CD4⁺ in the circulation with shared function and specificity with cytotoxic CD4⁺ TIL is indicative of a systemic anti-tumor response. Furthermore, the direct access may enable to circulating cytotoxic CD4⁺ and can greatly facilitate further mechanistic and therapeutic approaches involving these effectors. It can be noted that distinct subsets of circulating cytotoxic CD4⁺ express surface markers which can be stained such as KLRB1 and KLRG1, and furthermore that the KLRB1⁺ GZMK⁺ population may be selectively induced by PD-1 blockade. This may provide a direct route for prospective isolation of these cells, testing of their autologous anti-tumor killing activity across patients, and methods for ex vivo enhancement of their activity whether by various culture methods or cell engineering. Whether PD-1 blockade can be combined with other interventions (whether additional immune-based therapies in vivo to target the immunosuppressive milieu of bladder tumors or ex vivo manipulations), this suggests that monitoring of these specific cytotoxic CD4⁺ in addition to conventional cytotoxic CD8⁺ can provide a more complete view of how anti-tumor efficacy can be being enhanced with therapy. Sampling of the circulating cytotoxic CD4⁺ compartment could also represent a means of non-invasively monitoring the status of anti-tumor responses in situ, whether in the setting of known localized disease or as a possible diagnostic for occult disease.

Finally, an important unknown in determining which cytotoxic effector populations are therapeutically useful is understanding what antigens these cells are recognizing. The experimental described herein indicate that although the circulating CD4⁺ repertoire is generally quite diverse, the antigenic repertoire of cytotoxic CD4⁺ is restricted and shared between intratumoral and circulating compartments, with clonal expansion of a specific subset of these populations in situ. This expansion may be in response to antigens found within the tumor microenvironment, and if so this would narrow, the parameter space for possible antigens being recognized in light of restriction of the TCR repertoire in these cells.

### Example 13

### Reagents and Methods for Sorting CD4⁺ T Cells

This Examples describes general methods and reagents for sorting CD4⁺ T cells.

Required reagents included (1) SORT Buffer (500 ml) stored at 4 C consisting of PBS (500 ml), 2% heat inactivated FBS (10 ml), 1 mM EDTA (2 ml of 500 mM stock) and 1% pen/Strep; (2) BD Horizon Brilliant Stain buffer (BD, Cat. No. 56379) stored at 4 C; (3) UltraComp eBeads (Invitrogen Cat No. 01-2222-42) stored at 4 C; (4) Flow antibodies (store at 4 C, quick spin all antibodies and reagents in tubes before opening); (5) Draq7 (Biolegend) (store at 4 C); (6) collection medium: RF-10C.

The general methods include the following steps.
Thaw tumor cells according to best practice into complete media or use freshly digested tumor cells.
Thaw tumor cells according to best practice into complete media or use freshly digested tumor cells.
Wash 1× with FACs.
Resuspend cells in antibody premix below, mix and incubate in the dark on ice for 30 mins.
Wash 2× with 2 ml of FACs buffer.
Resuspend in approximately 2⁻⁶ cells/ml of FACs buffer. Pass cells through blue caps. (Spin down briefly and resuspend cells in tube if the sample do not pass through the blue cap).
Dilute Draq7 (AF700 channel) stock 10× in PBS. Add 10 ul per 500 ul sample (1:500 dilution of stock).
Compensation with single color controls is carried out.

Gating strategy: FSC-A vs SSC-A: large lymphocyte gate minus debris; FSC-W vs FSC-A: single cell gate 1; SSC-W vs SSC-A: single cell gate 2; Draq7 vs CD45; Gate Draq7⁻ CD45+; CD8 vs CD4: Gate CD8⁺CD4⁻ and CD4⁺CD8⁻; From CD4⁺CD8− plot CD127 vs CD25: Gate all cells excluding CD25+CD127low.

Sort CD4⁺ (minus CD25+CD127low), CD8⁺ and Tregs into FACs tube containing 300 ul RF-10C2 until sample is empty. Keep on ice.

All centrifugation is carried out at 450 g for 5 minutes unless otherwise indicated. All antibody tubes were mixed and spined before opening.

TABLE 2
Specifications for FAC sorting.

| Detector | Long Pass | Bandpass | | | | | | Opt. Vol. |
|---|---|---|---|---|---|---|---|---|
| Designation | Filter | Filter | Fluor | Marker | Clone | Company | Cat# | (ul) |
| Violet D | 570 | 605/40 | BV605 | CD25 | BC96 | Biolegend | 302632 | 2.5 |
| Violet A | 750 | 780/60 | BV786 | CD127 | A019D5 | Biolegend | 351330 | 2.5 |
| Violet F | NA | 450/50 | BV421 | CD4 | OKT4 | Biolegend | 317434 | 2 |
| Violet C | 635 | 670/30 | BV650 | CD3 | UCHT1 | Biolegend | 300468 | 2 |
| UV C | NA | 379/28 | BUV395 | CD45 | H130 | BD | 563792 | 2 |
| Red C | NA | 670/30 | AF647 | CD8 | SK1 | Biolegend | 344726 | 2 |
| Red B | 695 | 730/45 | Draq7 | Dead | | | | Add later |
| Brilliant buffer | | | | | | | | 50 |
| FACs buffer | | | | | | | | 37 |

### Example 14

### General Culturing Methods

This Examples describes general culturing methods.

Required materials included: (1) Human T cell medium (T cell expansion medium (STEM Cell; Cat 10981)+10% human AB serum+1% antibiotics); (2) Recombinant Human IL-2 (Peprotech Cat 200-02); (3) Dynabeads™ Human T-Activator (Design for expansion of antigen-specific T cells)(Gibco (11162D)).

The general culturing methods include the following steps, which may vary by sample:
Mix sorted CD4 (minus Tregs) and CD8 and seed into one 96-well U bottom.
Culture in T cell medium supplement with 200 IU/ml in 96 well U bottom.
Add Dynabeads (See attached protocol for recommendation usage).
After mixing, quick spin (30 secs) to make sure beads and T cells are in contact.
In about a 1 week, observe T cells forming a plaque at the bottom of the wells.
Passage to a new well (1 to 2 wells). Each well contains half original medium and half fresh medium
If the cells grow well, passage to bigger wells (e.g., 48 or 24 wells plate).
After 1-2 weeks, if the cells grow really well, expand TILs by increasing IL-2 concentration of 2000 IU/ml.
When expanding, keep half of the medium and add another half of fresh medium because when T cells expand, they secrete a lot of IL-2 in an autocrine fashion.
When TILs are in 2000 IU/ml, they usually grow very fast. Medium may be changed every 48 hours.

Generally, it may take about 1-2 months to obtain sufficient TILs.

Important steps for controlling the growth rate of TILs includes IL-2 concentration. It certain cases, no more than 2000 IU/ml may be added. Note: For Dynabeads, the protocol recommends to add additional dynabeads only if TILs stop to grow.

Cells were stained as described previously and sorted CD4 (minus Tregs), CD8 and Tregs (if needed).

Tumor cells can be also sorted as live (Draq7−) and CD45− into RPMI containing 10% heat inactivated FBS.

### Example 15

### Cell Killing Assay

This Examples describes a general cell killing assay.

Required materials included: (1) IncuCyte Red Cytotoxicity Reagent (EssenBioscience Cat #4632); (2) IncuCyte Green Rapid labeling (EssenBioscience Cat #46705); (3) Flat bottom 96 well tissue culture plate (Falcon 353072); (4) Medium: PBS.

A general cell killing assay include the following steps:
Count cells using the hemocytometer.
Label sorted autologous tumor cells by incubating of Green rapid reagent for 20 minutes at 37° C. (10⁻⁵ cells/ml, 0.03 mM final).
Wash tumor cells with 1× with PBS and resuspend in a volume of medium to give 3000 cells per 100 ul.
Incubate 100 ul of tumor cells (3000 cells) with 100 ul of CD4 or CD8 per well (with or without Tregs) in the ratio of 1:40 or 1:20 (tumor to TILs).
Add 0.5 ul of Red cytotoxicity reagent per well and mix. Quick spin at 450 g for 30 s.
Cells were imaged with the IncuCyte for 8 h with images taken at every ½h interval.

FIGS. 14A-14C show results of an exemplary killing assay performed with isolated cytotoxic CD4⁺ cells and cytotoxic CD8⁺ cells.

### Example 16

### Additional Methods and Techniques

### Tissue Processing

Peripheral blood mononuclear cells (PBMCs) and tissues were obtained from patients with localized bladder transitional cell carcinoma (TCC) who either received 1-3 doses of neoadjuvant atezolizumab as part of an ongoing clinical trial, or standard of care treatments including chemotherapy (gemcitabine/carboplatin) or no systemic therapy prior to planned cystectomy. Surgical specimens were obtained fresh from the operating field, and dissected in surgical pathology where grossly apparent tumor or adjacent bladder not grossly affected by tumor (“non-malignant”) were isolated, minced, and transported at room temperature immersed in L15 media with 15 mM HEPES and 600 mg % glucose. Once received, these were digested using Liberase TL as well as mechanical dissociation with heat (gentleMACS) using standard protocols. Single cell suspensions were obtained and counted for viability before staining for FACS.

### Flow Cytometry—FACS

PBMCs were thawed into FACS wash (PBS 2% BSA) and washed twice with FACS wash. Samples were stained with designated panels for 20 minutes at 4° C. and washed twice with FACS wash. Cells requiring intracellular staining were fixed and permeabilized with BD Cytofix/Cytoperm buffer (Cat #554722) according to the manufacturer's protocol. Intracellular staining with antibodies was carried out for 30 minutes at 4° C. and washed twice with FACS wash. Samples were acquired on a FACSAria Fusion (Becton Dickinson) using FACSDiva software with single channel compensation controls acquired on the same day. Data was analyzed off-line using FlowJo analysis software (FlowJo, LLC). Absolute counts (per ml of blood) for each immune subset is calculated by multiplying the percentage of each subset with the preceding parent subset and with the absolute lymphocyte count quantitated on the day of blood drawn.

### Single-Cell RNA Sequencing

Droplet-based single-cell RNA sequencing (dscRNAseq) was performed using the 10× Genomics Chromium Single Cell 3′ platform, version 1, according to manufacturer's instructions. CD3⁺CD4⁺ and CD3⁺CD8⁺ T cells were sorted from digested tumor and non-malignant tissues, or Ficoll-purified and previously cryopreserved PBMCs, into 500 μl of PSA/0.04% BSA for loading onto 10×. Following library preparation, sequencing was performed on an Illumina HiSeq 2500 (Rapid Run mode). Paired samples from the same experiment and patient were processed in parallel during library preparation, and sequenced on the same flowcell to minimize batch effects.

### Expression Analysis

After 10× sequencing data was processed through the Cell Ranger pipeline (version 1.1) with default settings, filtered gene-barcode matrices for single tumors were processed in Seurat (version 2.2.1, Rahul Satija lab, New York Genome Center), essentially by following the Guided Clustering Tutorial at “satijalab.org/seurat/pbmc3k_tutorial.html” and the CCA-Alignment Tutorial found at “satijalab.org/seurat/immune_alignment.html.” Cells that expressed fewer than 150 genes were filtered out and genes that were expressed in fewer than 5 cells. Next, the gene expression measurements for each cell was non-malignantized by the total expression, multiplied that total expression by a scale factor of 10,000, and log-transformed the result. Further, the non-malignantized dataset was scaled to remove confounding sources of variation by regressing out the signals driven by percent of mitochondrial gene expression and number of UMIs.

Multiple Canonical Correlation Analysis (MCCA) was then used to dimensionally reduce our dataset to 30 dimensions and align our dataset before further analysis. As the inputs to this algorithm, the obtained dataset was first filtered down to 1168 genes (CD4⁺ tissue), 1171 genes (CD8⁺ tissue), 1661 genes (CD4⁺ blood), or 1943 genes (CD8⁺ blood), which were found in the following way: for each “population,” which was defined as subset of the dataset consisting of a patient and tissue type, the 250 top variable genes were identified and the union of all of these genes was taken to create the input gene list. After examining the Metagene Bicorrelation Plot, a drop off in signal after around CC20 was observed, and so CC 1-20 were chosen for the alignment, for which tissue was chosen as the grouping variable.

To discover subtle differences among our cells, KNN graph-based Louvain clustering was performed. For CD4⁺ and CD8⁺ TIL, a resolution of 1.2 in Seurat's “FindClusters” command was used; for CD4⁺ and CD8⁺ from blood, analysis was done with resolution 0.9. The lower bound for resolution chosen for clustering was based on whether the minimum number of known phenotypic categories for CD4⁺ and CD8⁺ TIL were represented and also based on iterative comparison with parallel FACS staining which validated expression of markers within specific clusters, while the upper bound was informed by the presence of clusters with minimal numbers of cells which would indicate overclustering. t-Stochastic Neighbor Embedding (tSNE) plots was used for visualization purposes.

Seurat's “FindConservedMarkers” command was next used to run differential expression analysis between each cluster and a CCR7-high central memory cluster and identify expression markers that define a given cluster regardless of tissue type or timepoint (for blood). Significance was determined by non-parametric Wilcoxon rank sum test, with adjusted p value determined by Bonferroni correction. Heatmaps displaying conserved marker genes for each cluster were corrected across patients by fitting a linear model to remove sample-specific means. The gene lists were then compared to known literature to label the clusters, as well as using SingleR to map the expression signature for each cluster to the best correlated candidate immune reference signature, using the Human Primary Cell Atlas, Blueprint, and Encode microarray and RNAseq references described within.

Differential expression testing between tumor and non-malignant compartments was done with single cell expression data in a similar fashion; testing between tumor and non-malignant compartments was restricted to samples that had paired cells available from both compartments, Differential expression testing between anti-PD-L1-treated and untreated samples (excluding the chemotherapy sample) were done using pseudobulk representations for each sample and DESeq2 after filtering out genes with fewer than 100 reads.

### TCR Analysis

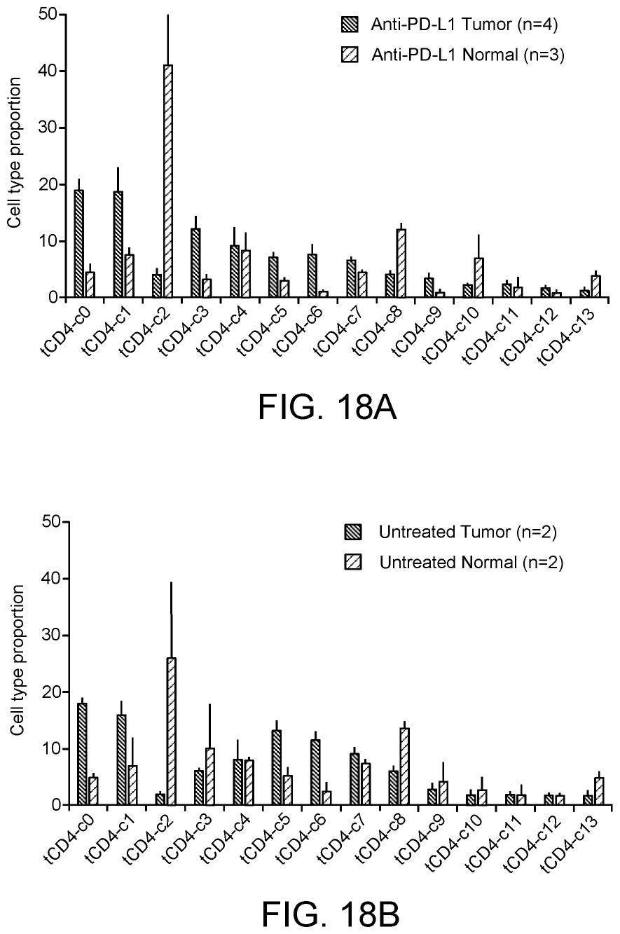

TRA and TRB CDR3 nucleotide reads were demultiplexed by matching reads to 10× barcodes from cells with existing expression data that passed filtering in the Cell Ranger pipeline, excluding cell barcodes that overlapped between multiple samples. Following demultiplexing of the TRA and TRB CDR3s, reads were aligned against known TRA/TRB CDR3 sequences then assembled into clonotype families using miXCR with similar methodologies to a previous study. For any given 10× barcode, the most abundant TRA or TRB clonotype was accepted for further analysis if 2 TRA or TRB clonotypes were equally abundant for a given 10× barcode, the clonotype with the highest sequence alignment score was used for further analysis. Detailed sequencing statistics and saturation analysis are provided in FIGS. 18A-18B. Only cells with paired TRA and TRB were used for further downstream analysis. Analysis utilizing TCR data only (number of unique cells sharing a specific TRA/TRB clonotype sequence, Gini coefficient) utilized cells both with and without a specific functional population that had been assigned by clustering. Analysis involving both TCR clonotype and function was restricted to cells with both a mapped TRA/TRB and a functional population from clustering. One sample (anti-PD-L1 A non-malignant) was incorporated in clustering and differential expression analyses but did not have corresponding TCR sequencing available.

To determine the enrichment of shared clonotypes between clusters, permutation tests were performed by randomly shuffling the cluster identities from all aggregated cells with paired TRA and TRB a total of 5,000 times and then generating a null hypothesis by counting the number of shared TCR clonotypes between clusters. Shuffling was confined to cells from blood, or to cells from tissue (tumor or non-malignant tissue). Empirical p-values were calculated by comparing the observed number of shared TCR clones and those by the null hypothesis to determine significance. Specifically, the probability of obtaining the observed number (or greater) of shared TCR clones by chance was calculated as 1—the cumulative distribution function for that pair of populations, based on the mean and standard deviation of the randomly shuffled distribution. The level of significance for this analysis was set at 0.01.

### Tumor Infiltrating Lymphocyte (TIL) Isolation and Culturing

Single-cell suspensions from processed and digested bladder tumors were viably frozen at −80 C and stored prior to culture setup. To sort the tumor-infiltrating lymphocytes, frozen cancer cell aliquots were thawed, washed once with PBS, and counted by Vicell. Cells were subsequent stained and sorted by FACS. CD4 TIL (Draq7⁻CD45⁺CD3⁺CD4⁺ that were not CD25⁺CD127^{lo}) and CD8 TIL (Draq7⁻CD45⁺CD3⁺CD8⁺) were sorted into ImmunoCult XF complete medium (Medium+10% FCS+1% penicillin/streptomycin; STEMCELL Technologies #10981). T cells were pooled together for culturing. After centrifugation, T cells were suspended in ImmunoCult XF complete medium, and Dynabeads (Gibco #11162D) were added to the culture per manufacturer's protocol. T cells were cultured in 96 well U-bottom plates, and briefly centrifuged to ensure cell contact with Dynabeads. T cell expansion was managed in two phases. For the first week of T cell expansion, TILs were maintained with ImmunoCult XF complete medium+200 IU/ml of human recombinant IL-2 (Peprotech #200-02). From the second week onward, IL-2 concentration was gradually increased from 200 IU/ml to 2000 IU/ml based on cell growth kinetics (which varied by patient sample). T cells were harvested between 5-8 weeks for functional killing assays.

### Cytotoxic T Lymphocyte (CTL) Killing Assay

After expansion, TILs were again sorted for either CD4 or CD8 as distinct effector populations. Primary cancer cells from frozen aliquots were freshly thawed and sorted on CD45-Draq7⁻ as target cells. To achieve various effector-to-target (E:T) ratios, 3000 cancer cell targets were suspended in ImmunoCult XF complete medium and seeded into each well. Different ratios of TILs were serially diluted and added to the corresponding wells. Each well contained 200 μl of medium supplemented with 0.25 μl of IncuCyte Red Cytotoxicity Reagent (Essen Bioscience #4632). For MHCI and MHCII blockade, 10 μg of blocking antibody was added into wells containing cancer cells and cultured at 37 C for 1 hour prior to co-culture with TILs. Cell culture was monitored by the IncuCyte Zoom system (EssenBioscience) at 15-30 minute intervals for a total of 12-24 hours. 2 independent experiments were performed with co-culture of CD4 and CD8 effectors with autologous tumor; results from 1 experiment are shown. Analysis was performed using the IncuCyte Zoom software. Red fluorescent images were background subtracted using a tophat filter with radius of 10 μm, and objects with a subtracted intensity of greater than 15 units were considered for further analysis. Tumor cells were larger than TIL based on inspection of wells with tumor cells alone or free TILs in wells containing TILs; based on this, the number of dying tumor cells per mm² was determined using a minimum area threshold of 75 μm², and in separate analyses the number of dying single TILs in wells containing TILs was determined using a minimum area threshold of 10 μm² and maximum area threshold of 65 μm². All numbers were normalized to the number at the start of the experiment. Out of focus frames were discarded, as were any wells where the first timeframe was out of focus precluding accurate normalization.

### Pseudotime Analysis

Pseudotime analysis using whole-transcriptome scRNAseq data was performed using Monocle2.

### Statistics

Specific statistical tests used for comparisons are described in the text. The chemotherapy sample was included in unbiased clustering, testing for conserved marker genes and tumor vs non-malignant testing, but was excluded from analyses of treatment effect (anti-PD-L1 vs untreated). For multiple testing correction, the Benjamini-Hochberg method was used with a false discovery rate <0.1 as implemented in the p.adjust function within the stats package within R.

### REFERENCES

1. Martincorena I, Campbell P J. Somatic mutation in cancer and normal cells. Science. 2015 Sep. 25; 349(6255):1483-9.
2. Cancer Genome Atlas Research Network. Comprehensive genomic characterization defines human glioblastoma genes and core pathways. Nature. 2008 Oct. 23; 455(7216):1061-8.
3. Hargadon K M, Johnson C E, Williams C J. Immune checkpoint blockade therapy for cancer: An overview of FDA-approved immune checkpoint inhibitors. Int Immunopharmacol. 2018 Jul. 2; 62:29-39.
4. Tirosh I, Izar B, Prakadan S M, Wadsworth M H 2nd, Treacy D, Trombetta J J, Rotem A, Rodman C, Lian C, Murphy G, Fallahi-Sichani M, Dutton-Regester K, Lin J R, Cohen O, Shah P, Lu D, Genshaft A S, Hughes T K, Ziegler C G, Kazer S W, Gaillard A, Kolb K E, Villani A C, Johannessen C M, Andreev A Y, Van Allen E M, Bertagnolli M, Sorger P K, Sullivan R J, Flaherty K T, Frederick D T, Jane-Valbuena J, Yoon C H, Rozenblatt-Rosen O, Shalek A K, Regev A, Garraway L A. Dissecting the multicellular ecosystem of metastatic melanoma by single-cell RNA-seq. Science. 2016 Apr. 8; 352(6282):189-96.
5. Philip M, Fairchild L, Sun L, Horste E L, Camara S, Shakiba M, Scott A C, Viale A, Lauer P, Merghoub T, Hellmann M D, Wolchok J D, Leslie C S, Schietinger A. Chromatin states define tumour-specific T cell dysfunction and reprogramming. Nature. 2017 May 25; 545(7655):452-456.
6. Ayers M, Lunceford J, Nebozhyn M, Murphy E, Loboda A, Kaufman D R, Albright A, Cheng J D, Kang S P, Shankaran V, Piha-Paul S A, Yearley J, Seiwert T Y, Ribas A, McClanahan T K. IFN-γ-related mRNA profile predicts clinical response to PD-1 blockade. J Clin Invest. 2017 Aug. 1; 127(8):2930-2940.
7. Herbst R S, Soria J C, Kowanetz M, Fine G D, Hamid O, Gordon M S, Sosman J A, McDermott D F, Powderly J D, Gettinger S N, Kohrt H E, Horn L, Lawrence D P, Rost S, Leabman M, Xiao Y, Mokatrin A, Koeppen H, Hegde P S, Mellman I, Chen D S, Hodi F S. Predictive correlates of response to the anti-PD-L1 antibody MPDL3280A in cancer patients. Nature. 2014 Nov. 27; 515(7528):563-7.
8. Tumeh P C, Harview C L, Yearley J H, Shintaku I P, Taylor E J, Robert L, Chmielowski B, Spasic M, Henry G, Ciobanu V, West A N, Carmona M, Kivork C, Seja E, Cherry G, Gutierrez A J, Grogan T R, Mateus C, Tomasic G, Glaspy J A, Emerson R O, Robins H, Pierce R H, Elashoff D A, Robert C, Ribas A. PD-1 blockade induces responses by inhibiting adaptive immune resistance. Nature. 2014 Nov. 27; 515(7528):568-71.
9. Koshkin V S, Grivas P. Emerging Role of Immunotherapy in Advanced Urothelial Carcinoma. Curr Oncol Rep. 2018 Apr. 11; 20(6):48.
10. Mariathasan S, Turley S J, Nickles D, Castiglioni A, Yuen K, Wang Y, Kadel E E III, Koeppen H, Astarita J L, Cubas R, Jhunjhunwala S, Banchereau R, Yang Y, Guan Y, Chalouni C, Ziai J, senbabaoglu Y, Santoro S, Sheinson D, Hung J, Giltnane J M, Pierce A A, Mesh K, Lianoglou S, Riegler J, Carano R A D, Eriksson P, Hoglund M, Somarriba L, Halligan D L, van der Heijden M S, Loriot Y, Rosenberg J E, Fong L, Mellman I, Chen D S, Green M, Derleth C, Fine G D, Hegde P S, Bourgon R, Powles T. TGFβ attenuates tumour response to PD-L1 blockade by contributing to exclusion of T cells. Nature. 2018 Feb. 22; 554(7693):544-548.
11. Zheng G X, Terry J M, Belgrader P, Ryvkin P, Bent Z W, Wilson R, Ziraldo S B, Wheeler T D, McDermott G P, Zhu J, Gregory M T, Shuga J, Montesclaros L, Underwood J G, Masquelier D A, Nishimura S Y, Schnall-Levin M, Wyatt P W, Hindson C M, Bharadwaj R, Wong A, Ness K D, Beppu L W, Deeg H J, McFarland C, Loeb K R, Valente W J, Ericson N G, Stevens E A, Radich J P, Mikkelsen T S, Hindson B J, Bielas J H. Massively parallel digital transcriptional profiling of single cells. Nat Commun. 2017 Jan. 16; 8:14049.
12. Butler A, Hoffman P, Smibert P, Papalexi E, Satija R. Integrating single-cell transcriptomic data across different conditions, technologies, and species. Nat Biotechnol. 2018 June; 36(5):411-420.
13. Blondel V D, Guillaume J-L, Lambiotte R, Lefebvre E. Fast unfolding of communities in large networks. J. Stat. Mech. 2008:P10008.
14. van der Maaten U P, Hinton G E. Visualizing data using t-SNE. J. Machine Learning Res.

2008 November; 9:2579-2605.
15. Zheng C, Zheng L, Yoo J K, Guo H, Zhang Y, Guo X, Kang B, Hu R, Huang J Y, Zhang Q, Liu Z, Dong M, Hu X, Ouyang W, Peng J, Zhang Z. Landscape of Infiltrating T Cells in Liver Cancer Revealed by Single-Cell Sequencing. Cell. 2017 Jun. 15; 169(7):1342-1356.e16.
16. Plitas G, Konopacki C, Wu K, Bos P D, Morrow M, Putintseva E V, Chudakov D M, Rudensky A Y. Regulatory T Cells Exhibit Distinct Features in Human Breast Cancer. Immunity. 2016 Nov. 15; 45(5):1122-1134.
17. De Simone M, Arrigoni A, Rossetti G, Gruarin P, Ranzani V, Politano C, Bonnal R J P, Provasi E, Sarnicola M L, Panzeri I, Moro M, Crosti M, Mazzara S, Vaira V, Bosari S, Palleschi A, Santambrogio L, Bovo G, Zucchini N, Totis M, Gianotti L, Cesana G, Perego R A, Maroni N, Pisani Ceretti A, Opocher E, De Francesco R, Geginat J, Stunnenberg H G, Abrignani S, Pagani M. Transcriptional Landscape of Human Tissue Lymphocytes Unveils Uniqueness of Tumor-Infiltrating T Regulatory Cells. Immunity. 2016 Nov. 15; 45(5):1135-1147.
18. Schmidt M, Weyer-Elberich V, Hengstler J G, Heimes A S, Almstedt K, Gerhold-Ay A, Lebrecht A, Battista M J, Hasenburg A, Sahin U, Kalogeras K T, Kellokumpu-Lehtinen P L, Fountzilas G, Wirtz R M, Joensuu H. Prognostic impact of CD4-positive T cell subsets in early breast cancer: a study based on the FinHer trial patient population. Breast Cancer Res. 2018 Feb. 26; 20(1):15.
19. Gu-Trantien C, Loi S, Garaud S, Equeter C, Libin M, de Wind A, Ravoet M, Le Buanec H, Sibille C, Manfouo-Foutsop G, Veys I, Haibe-Kains B, Singhal S K, Michiels S, Rothé F, Salgado R, Duvillier H, Ignatiadis M, Desmedt C, Bron D, Larsimont D, Piccart M, Sotiriou C, Willard-Gallo K. CD4⁺ follicular helper T cell infiltration predicts breast cancer survival. J Clin Invest. 2013 July; 123(7):2873-92.
20. Gu-Trantien C, Migliori E, Buisseret L, de Wind A, Brohée S, Garaud S, Noël G, Dang Chi V L, Lodewyckx J N, Naveaux C, Duvillier H, Goriely S, Larsimont D, Willard-Gallo K. CXCL13-producing TFH cells link immune suppression and adaptive memory in human breast cancer. JCI Insight. 2017 Jun. 2; 2(11). pii: 91487.
21. Wei Y, Lin C, Li H, Xu Z, Wang J, Li R, Liu H, Zhang H, He H, Xu J. CXCL13 expression is prognostic and predictive for postoperative adjuvant chemotherapy benefit in patients with gastric cancer. Cancer Immunol Immunother. 2018 February; 67(2):261-269.
22. Zhang L, Yu X, Zheng L, Zhang Y, Li Y, Fang Q, Gao R, Kang B, Zhang Q, Huang J Y, Konno H, Guo X, Ye Y, Gao S, Wang S, Hu X, Ren X, Shen Z, Ouyang W, Zhang Z. Lineage tracking reveals dynamic relationships of T cells in colorectal cancer. Nature. 2018 December; 564(7735):268-272.
23. Krensky A M, Clayberger C. Biology and clinical relevance of granulysin. Tissue Antigens. 2009 Mar.; 73(3):193-8.
24. Medley Q G, Kedersha N, O'Brien S, Tian Q, Schlossman S F, Streuli M, Anderson P. Characterization of GMP-17, a granule membrane protein that moves to the plasma membrane of natural killer cells following target cell recognition. Proc Natl Acad Sci USA. 1996 Jan. 23; 93(2):685-9.
25. Löfroos A B, Kadivar M, Resic Lindehammer S, Marsal J. Colorectal cancer-infiltrating T lymphocytes display a distinct chemokine receptor expression profile. Eur J Med Res. 2017 Oct. 11; 22(1):40.
26. Parsonage G, Machado L R, Hui J W, McLarnon A, Schmaler T, Balasothy M, To K F, Vlantis A C, van Hasselt C A, Lo K W, Wong W L, Hui E P, Chan A T, Lee S P. CXCR6 and CCR5 localize T lymphocyte subsets in nasopharyngeal carcinoma. Am J Pathol. 2012 March; 180(3):1215-22.
27. Oldham K A, Parsonage G, Bhatt R I, Wallace D M, Deshmukh N, Chaudhri S, Adams D H, Lee S P. T lymphocyte recruitment into renal cell carcinoma tissue: a role for chemokine receptors CXCR3, CXCR6, CCR5, and CCR6. Eur Urol. 2012 February; 61(2):385-94.
28. Duhen T, Duhen R, Montler R, Moses J, Moudgil T, de Miranda N F, Goodall C P, Blair T C, Fox B A, McDermott J E, Chang S C, Grunkemeier G, Leidner R, Bell R B, Weinberg A D. Co-expression of CD39 and CD103 identifies tumor-reactive CD8 T cells in human solid tumors. Nat Commun. 2018 Jul. 13; 9(1):2724.
29. Kurioka A, Walker L J, Klenerman P, Willberg C B. MAIT cells: new guardians of the liver. Clin Transl Immunology. 2016; 5:e98.
30. Aran D, Looney A P, Liu L, Wu E, Fong V, Hsu A, Chak S, Naikawadi R P, Wolters P J, Abate A R, Butte A J, Bhattacharya M. Reference-based analysis of lung single-cell sequencing reveals a transitional profibrotic macrophage. Nat Immunol. 2019 February; 20(2):163-172.
31. Bae J, Munshi A, Li C, Samur M, Prabhala R, Mitsiades C, Anderson K C, Munshi N C. Heat shock protein 90 is critical for regulation of phenotype and functional activity of human T lymphocytes and N K cells. J Immunol. 2013 Feb. 1; 190(3):1360-71.
32. Berges C, Kerkau T, Werner S, Wolf N, Winter N, Hünig T, Einsele H, Topp M S, Beyersdorf N. Hsp90 inhibition ameliorates CD4⁺ T cell-mediated acute Graft versus Host disease in mice. Immun Inflamm Dis. 2016 Oct. 10; 4(4):463-473.
33. Carlson C M, Endrizzi B T, Wu J, Ding X, Weinreich M A, Walsh E R, Wani M A, Lingrel J B, Hogquist K A, Jameson S C. Kruppel-like factor 2 regulates thymocyte and T-cell migration. Nature. 2006 Jul. 20; 442(7100):299-302.
34. Skon C N, Lee J Y, Anderson K G, Masopust D, Hogquist K A, Jameson S C. Transcriptional downregulation of S1pr1 is required for the establishment of resident memory CD8⁺ T cells. Nat Immunol. 2013 December; 14(12):1285-93.
35. Qiu X, Mao Q, Tang Y, Wang L, Chawla R, Pliner H A, Trapnell C. Reversed graph embedding resolves complex single-cell trajectories. Nat Methods. 2017 October; 14(10):979-982.
36. Ott P A, Hu Z, Keskin D B, Shukla S A, Sun J, Bozym D J, Zhang W, Luoma A, Giobbie-Hurder A, Peter L, Chen C, Olive O, Carter T A, Li S, Lieb D J, Eisenhaure T, Gjini E, Stevens J, Lane W J, Javeri I, Nellaiappan K, Salazar A M, Daley H, Seaman M, Buchbinder E I, Yoon C H, Harden M, Lennon N, Gabriel S, Rodig S J, Barouch D H, Aster J C, Getz G, Wucherpfennig K, Neuberg D, Ritz J, Lander E S, Fritsch E F, Hacohen N, Wu C J. An immunogenic personal neoantigen vaccine for patients with melanoma. Nature. 2017 Jul. 13; 547(7662):217-221.
37. Sahin U, Derhovanessian E, Miller M, Kloke B P, Simon P, Löwer M, Bukur V, Tadmor A D, Luxemburger U, Schrörs B, Omokoko T, Vormehr M, Albrecht C, Paruzynski A, Kuhn A N, Buck J, Heesch S, Schreeb K H, Müller F, Ortseifer I, Vogler I, Godehardt E, Attig S, Rae R, Breitkreuz A, Tolliver C, Suchan M, Martic G, Hohberger A, Sorn P, Diekmann J, Ciesla J, Waksmann O, Brück A K, Witt M, Zillgen M, Rothermel A, Kasemann B, Langer D, Bolte S, Diken M, Kreiter S, Nemecek R, Gebhardt C, Grabbe S, Höller C, Utikal J, Huber C, Loquai C, Türeci Ö. Personalized RNA mutanome vaccines mobilize poly-specific therapeutic immunity against cancer. Nature. 2017 Jul. 13; 547(7662):222-226.
38. Guo X, Zhang Y, Zheng L, Zheng C, Song J, Zhang Q, Kang B, Liu Z, Jin L, Xing R, Gao R, Zhang L, Dong M, Hu X, Ren X, Kirchhoff D, Roider H G, Yan T, Zhang Z. Global characterization of T cells in non-small-cell lung cancer by single-cell sequencing. Nat Med. 2018 July; 24(7):978-985.
39. Kitano S, Tsuji T, Liu C, Hirschhorn-Cymerman D, Kyi C, Mu Z, Allison J P, Gnjatic S, Yuan J D, Wolchok J D. Enhancement of tumor-reactive cytotoxic CD4⁺ T cell responses after ipilimumab treatment in four advanced melanoma patients. Cancer Immunol Res. 2013 October; 1(4):235-44.
40. Patil V S, Madrigal A, Schmiedel B J, Clarke J, O'Rourke P, de Silva A D, Harris E, Peters B, Seumois G, Weiskopf D, Sette A, Vijayanand P. Precursors of human CD4⁺ cytotoxic T lymphocytes identified by single-cell transcriptome analysis. Sci Immunol. 2018 Jan. 19; 3(19). pii: eaan8664.
41. Han A, Glanville J, Hansmann L, Davis M M. Linking T-cell receptor sequence to functional phenotype at the single-cell level. Nat Biotechnol. 2014 July; 32(7):684-92.
42. Love M I, Huber W, Anders S. Moderated estimation of fold change and dispersion for RNA-seq data with DESeq2. Genome Biol. 2014; 15(12):550.
43. Bolotin D A, Poslaysky S, Mitrophanov I, Shugay M, Mamedov I Z, Putintseva E V, Chudakov D M. MiXCR: software for comprehensive adaptive immunity profiling. Nat Methods. 2015 May; 12(5):380-1.
44. Zemmour D, Zilionis R, Kiner E, Klein A M, Mathis D, Benoist C. Single-cell gene expression reveals a landscape of regulatory T cell phenotypes shaped by the TCR. Nat Immunol. 2018 March; 19(3):291-301.

1. An ex vivo population of CD4⁺ T cells, wherein the CD4⁺ T cells express one or more markers selected from the group consisting of GZMA, GZMB, GZMH, GZMK, KLRB1, KLRD1, GNLY, NKG7, CCL4, CCL5, LTB, CXCR4, CXCR6, PRF1, KLRG1, LAG3, CXCL13, and combinations of any thereof, and wherein the CD4⁺ T cells have cytolytic capabilities.

2. The population of claim 1, wherein the CD4⁺ T cells express the immune checkpoint marker LAG3 but lack expression of one or more additional immune checkpoint markers.

3. The population of claim 2, wherein the one or more additional immune checkpoint markers is selected from the group consisting of IL2RA/CD25, TNFRSF4/OX40, TNFRSF9/4-1BB, TNFRSF18/GITR, CD278/ICOS, TIGIT, and combinations of any thereof.

4. The population of claim 1, wherein the CD4⁺ T cells further express heat shock proteins and/or IFN-gamma.

5. (canceled)

6. The population of claim 1, wherein the CD4⁺ T cells express a T cell receptor (TCR) comprising:
a TCR alpha CDR3 sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9; and
a TCR beta CDR3 sequence selected from the group consisting of SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, and SEQ ID NO: 19.

7. The population of claim 1, wherein the CD4⁺ T cells are obtained from a biological sample comprising bladder cancer cells.

8. The population of claim 1, wherein the CD4⁺ T cells are obtained from a biological sample comprising peripheral blood from an individual having or suspected of having bladder cancer.

9. The population of claim 1, wherein the CD4⁺ T cells have decreased cytolytic capabilities as compared to the CD4⁺ T cells which have been expanded ex vivo.

10. The population of claim 1, wherein the population is at least 50%, 60%, 70%, 80%, 90%, or 95% enriched for cytolytic CD4⁺ T cells.

11. The population of claim 1, wherein the CD4⁺ T cells are capable of killing autologous cancer cells.

12. A pharmaceutical composition for adoptive cell therapy, the composition comprising an ex vivo population of CD4⁺ T cells according to claim 1 and a pharmaceutically acceptable excipient.

13. (canceled)

14. The pharmaceutical composition of claim 12, wherein the population is at least 50%, 60%, 70%, 80%, 90%, or 95% enriched for CD4⁺ T cells with cytolytic capabilities.

15. A method for producing an ex vivo expanded population of CD4⁺ T cells with cytolytic capabilities, the method comprising:
(a) separating CD4⁺ T cells from a biological sample containing a mixture of different types of immune cells;
(b) culturing the separated CD4⁺ T cells in media containing IL-2 in an amount sufficient to promote the expansion of CD4⁺ T cells;
(c) splitting the cultured CD4⁺ T cells to promote the enrichment of CD4⁺ T cells thus producing an ex vivo expanded population of CD4⁺ T cells with cytolytic capabilities.

16. The method of claim 15, wherein IL-2 is used in the amount of about 1 IU/ml to about 2000 IU/ml.

17. (canceled)

18. The method of claim 15, wherein the ex vivo expanded population of CD4⁺ T cells with cytolytic capabilities express one or more markers selected from the group consisting of GZMA, GZMB, GZMH, GZMK, KLRB1, KLRD1, GNLY, NKG7, CCL4, CCL5, LTB, CXCR4, CXCR6, PRF1, KLRG1, LAG3, and CXCL13.

19. The method of claim 15, wherein the CD4⁺ T cells express the immune checkpoint marker LAG3 but lack expression of one or more additional immune checkpoint markers.

20. (canceled)

21. The method of claim 15, wherein the CD4⁺ T cells further express heat shock proteins and/or IFN-gamma.

22. (canceled)

23. A method for providing a cell therapy or for treating an individual having or suspected of having bladder cancer, the method comprising administering to the individual a composition comprising an effective amount of a population of cytolytic CD4⁺ T cells.

24.-25. (canceled)

26. The method of claim 23, wherein the administered composition inhibits the growth and/or proliferation of one or more bladder cancer cells.

27. The method of claim 23, wherein the population of cytolytic CD4⁺ T cells to be administered to the individual is an ex vivo population of cytolytic CD4⁺ T cells according to claim 1.

28.-31. (canceled)


SEQUENCE LISTING (19 sequences)

SEQ ID NO: 1 - protein, 14 aa, Artificial Sequence
  TCR alpha CDR3 sequence
CALRGTNTGNQFYF

SEQ ID NO: 2 - protein, 17 aa, Artificial Sequence
  TCR alpha CDR3 sequence
CAVEAGGVVGSARQLTF

SEQ ID NO: 3 - protein, 13 aa, Artificial Sequence
  TCR alpha CDR3 sequence
CAIIIQGAQKLVF

SEQ ID NO: 4 - protein, 12 aa, Artificial Sequence
  TCR alpha CDR3 sequence
CAFMKGNNDMRF

SEQ ID NO: 5 - protein, 14 aa, Artificial Sequence
  TCR alpha CDR3 sequence
CATADKGGAQKLVF

SEQ ID NO: 6 - protein, 12 aa, Artificial Sequence
  TCR alpha CDR3 sequence
CVVNGGGDKLIF

SEQ ID NO: 7 - protein, 14 aa, Artificial Sequence
  TCR alpha CDR3 sequence
CATENLQGAQKLVF

SEQ ID NO: 8 - protein, 16 aa, Artificial Sequence
  TCR alpha CDR3 sequence
CAPPRRVDSDGQKLLF

SEQ ID NO: 9 - protein, 12 aa, Artificial Sequence
  TCR alpha CDR3 sequence
CALNNAGNMLTF

SEQ ID NO: 10 - protein, 16 aa, Artificial Sequence
  TCR beta CDR3 sequence
CASSSAMVAGAYEQYF

SEQ ID NO: 11 - protein, 15 aa, Artificial Sequence
  TCR beta CDR3 sequence
CSARDPKGGDTEAFF

SEQ ID NO: 12 - protein, 15 aa, Artificial Sequence
  TCR beta CDR3 sequence
CASSQSRDRTYEQYF

SEQ ID NO: 13 - protein, 13 aa, Artificial Sequence
  TCR beta CDR3 sequence
CASSQGANQPQHF

SEQ ID NO: 14 - protein, 17 aa, Artificial Sequence
  TCR beta CDR3 sequence
CASSSILGEVATGELFF

SEQ ID NO: 15 - protein, 13 aa, Artificial Sequence
  TCR beta CDR3 sequence
CASSRTGGTEAFF

SEQ ID NO: 16 - protein, 13 aa, Artificial Sequence
  TCR beta CDR3 sequence
CASSQDPTYEQYF

SEQ ID NO: 17 - protein, 15 aa, Artificial Sequence
  TCR beta CDR3 sequence
CASSPTGTGIRGYTF

SEQ ID NO: 18 - protein, 18 aa, Artificial Sequence
  TCR beta CDR3 sequence
CASSLIPGQGATSYGYTF

SEQ ID NO: 19 - protein, 16 aa, Artificial Sequence
  TCR beta CDR3 sequence
CASSLARSFGLGEQFF